(19) United States
(12) Patent Application Publication
(10) Pub. No.: US 2023/0089784 A1
(43) Pub. Date: Mar. 23, 2023

(54) METHODS AND COMPOSITIONS FOR PRODUCTION OF GENETICALLY MODIFIED PRIMARY CELLS

(71) Applicant: Graphite Bio, Inc., South San Francisco, CA (US)

(72) Inventors: Beeke WIENERT, South San Francisco, CA (US); Rajiv SHARMA, South San Francisco, CA (US); Daniel DEVER, South San Francisco, CA (US); Aishwarya CHURI, South San Francisco, CA (US); Christopher BANDORO, South San Francisco, CA (US)

(21) Appl. No.: 17/719,236

(22) Filed: Apr. 12, 2022

Related U.S. Application Data
(60) Provisional application No. 63/173,859, filed on Apr. 12, 2021.

(51) Int. Cl. C12N 9/22; C12N 15/86; A61P 7/00; C12N 5/0789

(52) U.S. Cl. CPC C12N 9/22; C12N 15/86; A61P 7/00; C12N 5/0647; C12N 2800/80; C12N 2310/20

(57) ABSTRACT

Provided herein are compositions, methods, and systems, comprising a programmable nucleic acid-guided nuclease and sequence-diverged donor sequences. The compositions and methods described herein facilitate editing of a targeted locus using a diverged sequence encoding for a functional protein product.

### CROSS REFERENCE TO RELATED APPLICATIONS

This application claims the benefit of, and priority to, U.S. provisional patent application Ser. No. 63/173,859, filed on Apr. 12, 2021, which is hereby incorporated by reference herein in its entirety.

### SEQUENCE LISTING

The instant application contains a Sequence Listing which has been submitted electronically in ASCII format and is hereby incorporated by reference in its entirety. Said ASCII copy, created on Aug. 19, 2022, is named 58539-710_201_SL.txt and is 97,963 bytes in size.

### BACKGROUND OF THE INVENTION

Individuals with β-thalassemia trait show mild to no symptoms of disease while individuals with beta-thalassemia major show erythrocytoxicity due to an accumulation of unpaired α-globin chains contributing to the disease phenotype in patients. Therefore, one goal of gene therapy is to increase the amount of β-globin to at least 50% of the alpha-globin chains (imitating β-thalassemia trait) with the aim to reduce the amount of toxic unpaired α-globin chains and to generate sufficient amounts of functional hemoglobin (HbA, α₂β₂). Isolating the patient's own hematopoietic stem and progenitor cells (HSPCs) and introducing a functional HBB gene would be an ideal therapeutic strategy as these corrected cells would not be rejected by the patient upon reinfusion. Gene addition using lentiviral vectors stably transfers the HBB gene including introns and regulatory elements to HSPCs and has shown promising outcomes in the clinic. However, lentiviruses integrate semi-randomly which could activate neighboring genes resulting in oncogenesis or clonal expansion, and reaching high enough levels of β-globin expression from a lentiviral transgene remains a major challenge. The genetic elements that transcriptionally activate β-globin are well studied and it is known that the presence of an upstream enhancer (the locus control region, LCR), the β-globin promoter, β-globin introns and 3′ regulatory regions are necessary for efficient erythroid specific transcription. Hence, lentiviral transgenes must include those transcriptional elements in addition to the HBB gene sequence resulting in relatively large lentiviral cassettes which affects viral titers and transduction efficiencies in HSPCs. Consequently, there is a need for genome editing strategies that result in high enough levels of β-globin to ensure a full cure in these patients.

In relation to gene editing strategies more broadly, in instances where a targeted gene harbors one or more mutations, and correction or replacement of the mutant allele within its native locus is desired, donor polynucleotides encoding a wild-type functional copy of the targeted gene may be utilized. Ideally, HDR of the exogenous polynucleotide occurs only through the 5′ and 3′ homology arms that flank the donor gene, so that the entirety of the exogenous polynucleotide sequence between the homology arms is integrated into the targeted locus. However, where the donor gene shares high nucleotide sequence identity with the targeted mutant allele, undesired partial recombination events can lead to incomplete or unsuccessful integration of the entirety of the intended donor sequence. Compositions and methods are provided are provided herein to help avoid these outcomes.

### SUMMARY OF THE INVENTION

Provided herein in the present disclosure is a method of targeted integration of an exogenous polynucleotide sequence into a gene locus of a cell, the method comprising introducing into the cell: (a) a site-specific nuclease system capable of generating a double-strand break within the gene locus; (b) a recombinant vector comprising a donor polynucleotide, wherein the donor polynucleotide comprises: (i) the exogenous polynucleotide sequence which encodes a protein, wherein the exogenous polynucleotide sequence comprises at least one heterologous intron sequence or a portion thereof; and (ii) 5′ and 3′ homology arms flanking the exogenous polynucleotide sequence, wherein each homology arm is homologous to a portion of the gene locus; whereupon generation of the double-strand break within the gene locus by the site-specific nuclease system, the nucleic acid sequence of the donor polynucleotide is integrated into the gene locus by homology directed repair (HDR), resulting in exogenous production of the protein from the gene locus of the cell. In some embodiments, the exogenous polynucleotide sequence comprises 2, 3, 4, 5, or more heterologous intron sequences or portions thereof.

In some embodiments, the site-specific nuclease system comprises a CRISPR nuclease and a single guide RNA (sgRNA) capable of hybridizing to the gene locus. In some embodiments, the CRISPR nuclease is a Cas protein. In some embodiments, Cas protein is Cas9 or a high-fidelity variant thereof. In some embodiments, the sgRNA and the CRISPR nuclease are incubated together to form a ribonucleoprotein (RNP) complex prior to introducing into the cell. In some embodiments, the RNP complex is introduced into the cell before the recombinant vector. In some embodiments, the sgRNA comprises one or more chemically modified nucleotides. In some embodiments, the modified nucleotide is selected from the group consisting of: a 2′-O-methyl nucleotide, a 2′-O-methyl 3′-phosphorothioate nucleotide, and a 2′-O-methyl 3′-thioPACE nucleotide. In some embodiments, a 5′ end, a 3′ end, or a combination thereof of the modified sgRNA comprises a modified nucleotide.

In some embodiments, the vector is selected from the group consisting of viral vectors, plasmids, and ssDNAs. In some embodiments, the vector is an adeno-associated viral (AAV) vector. In some embodiments, the AAV vector is selected from the group consisting of: AAV1, AAV2, AAV3, AAV4, AAV3, AAV5, AAV6, AAV7, AAV8, AAV9, AAV10, AAV11 and AAV12. In some embodiments, the AAV vector is an AAV6 vector.

In some embodiments, exogenous production of protein from the gene locus of the cell is regulated by the native promoter sequence of the gene locus. In some embodiments, the cell is a primary cell. In some embodiments, the primary cell is a mammalian primary cell. In some embodiments, the primary cell is a human cell. In some embodiments, the primary cell is selected from the group consisting of a primary blood cell and a primary mesenchymal cell. In some embodiments, the primary cell is selected from the group consisting of a primary stem cell, primary progenitor cell, and primary somatic cell. In some embodiments, the stem cell selected from the group consisting of an embryonic stem cell, induced pluripotent stem cell, hematopoietic stem cell, mesenchymal stem cell, neural stem cell, and organ stem cell. In some embodiments, the progenitor cell is selected from the group consisting of a hematopoietic progenitor cell, a myeloid progenitor cell, a lymphoid progenitor cell, a multipotent progenitor cell, an oligopotent progenitor cell, and a lineage-restricted progenitor cell. In some embodiments, the somatic cell is selected from the group consisting of a fibroblast, a hepatocyte, a heart cell, a liver cell, a pancreatic cell, a muscle cell, a skin cell, a blood cell, a neural cell, and an immune cell. In some embodiments, the immune cell is selected from the group consisting of T lymphocyte (T cell), B lymphocyte (B cell), small lymphocyte, natural killer cell (NK cell), natural killer T cell, macrophage, monocyte, monocyte-precursor cell, eosinophil, neutrophil, basophils, megakaryocyte, myeloblast, mast cell and dendritic cell. In some embodiments, the primary cell is a CD34+ hematopoietic stem and progenitor cell (HSPC).

In some embodiments, the gene locus of the cell comprises one or more mutations associated with a disease or encodes an aberrant protein. In some embodiments, integration of the donor polynucleotide sequence corrects a mutation in the cell that is associated with a disease. In some embodiments, integration of the donor polynucleotide sequence replaces a mutant allele in the cell with a wild-type allele. In some embodiments, the disease is selected from the group consisting of a hemoglobinopathy, a viral infection, X-linked severe combined immune deficiency, Fanconi anemia, hemophilia, neoplasia, cancer, alpha-1 antitrypsin deficiency, amyotrophic lateral sclerosis, Alzheimer's disease, Parkinson's disease, cystic fibrosis, blood diseases and disorders, inflammation, immune system diseases or disorders, metabolic diseases, liver diseases and disorders, kidney diseases and disorders, muscular diseases and disorders, bone or cartilage diseases and disorders, neurological and neuronal diseases and disorders, cardiovascular diseases and disorders, pulmonary diseases and disorders, and lysosomal storage disorders.

In some embodiments, the gene locus of the cell is a Hemoglobin Subunit gene locus. In some embodiments, Hemoglobin Subunit gene is selected from the group consisting of the Hemoglobin Subunit Beta (HBB) gene, the Hemoglobin Subunit Alpha 1 (HBA1) gene, and the Hemoglobin Subunit Alpha 2 (HBA2) gene. In some embodiments, the Hemoglobin Subunit gene locus comprises one or more genetic mutations associated with a hemoglobinopathy. In some embodiments, the HSPC is isolated from a subject having a hemoglobinopathy. In some embodiments, the hemoglobinopathy is sickle cell disease, α-thalassemia, β-thalassemia, or δ-thalassemia.

In some embodiments, the at least one heterologous intron sequence or a portion thereof is derived from an intron sequence of a Hemoglobin Subunit gene selected from the group consisting of Hemoglobin Subunit Alpha 1 (HBA1) gene, Hemoglobin Subunit Beta (HBB), Hemoglobin Subunit Delta (HBD), and Hemoglobin Subunit Gamma 2 (HBG2). In some embodiments, the exogenous polynucleotide sequence encodes beta globin protein. In some embodiments, the exogenous polynucleotide sequence encodes alpha-1 antitrypsin protein. In some embodiments, the gene locus of the cell is CCR5. In some embodiments, the method is performed ex vivo.

In another aspect, provided herein in the present disclosure is a composition comprising a population of primary hematopoietic stem and progenitor cells (HSPCs) isolated from a subject, wherein one or more primary HSPCs of the population comprise: (a) a site-specific nuclease system capable of generating a double-strand break within a gene locus of the HSPC; and (b) a recombinant vector comprising a donor polynucleotide, wherein the donor polynucleotide comprises: (i) an exogenous polynucleotide sequence which encodes a protein, wherein the exogenous polynucleotide sequence comprises at least one heterologous intron sequence or a portion thereof; and (ii) 5′ and 3′ homology arms flanking the exogenous polynucleotide sequence, wherein each homology arm is homologous to a portion of the gene locus; whereupon generation of the double-strand break within the gene locus by the site-specific nuclease system, the nucleic acid sequence of the donor polynucleotide is integrated into the gene locus by homology directed repair (HDR), resulting in exogenous production of the protein from the gene locus of the cell. In some embodiments, the exogenous polynucleotide sequence comprises 2, 3, 4, 5, or more heterologous intron sequences or portions thereof.

In another aspect, provided herein in the present disclosure is a HBB donor polynucleotide comprising, in a 5′ to 3′ orientation: (a) a first Hemoglobin Subunit Beta (HBB) homology region comprising a nucleic acid sequence having at least 95% sequence identity to a first target region of the HBB gene; (b) a diverged HBB exon 1 region comprising a nucleic acid sequence having less than 95% sequence identity to exon 1 of the HBB gene, and which encodes an amino acid sequence encoded by exon 1 of the HBB gene; (c) a heterologous globin intron 1 region comprising a nucleic acid sequence having at least 95% sequence identity to intron 1, or a portion thereof, of a Hemoglobin Subunit gene; (d) a diverged HBB exon 2 region comprising a nucleic acid sequence having less than 95% sequence identity to exon 2 of the HBB gene, and which encodes an amino acid sequence encoded by exon 2 of the HBB gene; (e) a heterologous globin intron 2 region comprising a nucleic acid sequence having at least 95% sequence identity to intron 2, or a portion thereof, of a Hemoglobin Subunit gene; (f) a diverged HBB exon 3 region comprising a nucleic acid sequence having less than 95% sequence identity to exon 3 of the HBB gene, and which encodes an amino acid sequence encoded by exon 3 of the HBB gene; and (g) a second HBB homology region comprising a nucleic acid sequence having at least 95% sequence identity to a second target region of the HBB gene, wherein the second target region is positioned 3′ to the first target region in the HBB gene; wherein homology directed repair (HDR)-mediated integration of the HBB donor polynucleotide sequence into an HBB locus results in exogenous expression of beta globin protein from the HBB locus.

In some embodiments, the HBB donor polynucleotide further comprises a polyadenylation signal sequence positioned between the diverged HBB exon 3 and the second HBB homology region. In some embodiments, the polyadenylation signal sequence is selected from the group consisting of a polyadenylation signal sequence from bovine growth hormone (bGH), human growth hormone (hGH), rabbit beta globin (RbGlob), a synthetic poly A sequence based on rabbit beta globin poly A (SynthRbGlob) and Simian Virus 40 (SV40).

In some embodiments, the first target region of the HBB gene comprises the nucleic acid sequence of SEQ ID NO: 19 or SEQ ID NO: 69. In some embodiments, the second target region of the HBB gene comprises the nucleic acid sequence of SEQ ID NO: 20 or SEQ ID NO: 70. In some embodiments, the diverged HBB exon 1 region comprises a nucleic acid sequence having between 60% and 90% sequence identity to exon 1 of the HBB gene. In some embodiments, the diverged HBB exon 1 region comprises the nucleic acid sequence of SEQ ID NO: 35. In some embodiments, the diverged HBB exon 2 region comprises a nucleic acid sequence having between 57% and 90% sequence identity to exon 2 of the HBB gene. In some embodiments, the diverged HBB exon 2 region comprises the nucleic acid sequence of SEQ ID NO: 36. In some embodiments, the diverged HBB exon 3 region comprises a nucleic acid sequence having between 62% and 90% sequence identity to exon 3 of the HBB gene. In some embodiments, the diverged HBB exon 3 region comprises the nucleic acid sequence of SEQ ID NO: 37.

In some embodiments, the heterologous globin intron 1 region comprises a nucleic acid sequence having at least 95% sequence identity to intron 1, or a portion thereof, of a Hemoglobin Subunit gene selected from the group consisting of Hemoglobin Subunit Alpha 1 (HBA1), Hemoglobin Subunit Beta (HBB), Hemoglobin Subunit Delta (HBD), and Hemoglobin Subunit Gamma 2 (HBG2). In some embodiments, the Hemoglobin Subunit gene is HBG2. In some embodiments, the heterologous globin intron 1 region comprises the nucleic acid sequence of SEQ ID NO: 11. In some embodiments, the heterologous globin intron 2 region comprises a nucleic acid sequence having at least 95% sequence identity to intron 2, or a portion thereof, of a Hemoglobin Subunit gene selected from the group consisting of Hemoglobin Subunit Alpha 1 (HBA1), Hemoglobin Subunit Beta (HBB), Hemoglobin Subunit Delta (HBD), and Hemoglobin Subunit Gamma 2 (HBG2). In some embodiments, the Hemoglobin Subunit gene is HBG2. In some embodiments, the heterologous globin intron 2 region comprises the nucleic acid sequence of SEQ ID NO: 12. In some embodiments, the heterologous globin intron 2 region comprises a truncated intron 2 of a Hemoglobin Subunit gene, wherein the truncation comprises deletion of nucleotides 21-437 and 513-834 of the intron. In some embodiments, the truncated intron 2 comprises a truncated HBG2 intron 2 nucleic acid sequence. In some embodiments, the truncated HBG2 intron 2 nucleic acid sequence comprises the nucleic acid sequence of SEQ ID NO: 78. In some embodiments, the donor polynucleotide comprises a nucleic acid sequence selected from the group consisting of SEQ NO: 88, SEQ NO: 89, SEQ NO: 90 and SEQ NO: 91.

In some embodiments, exogenous expression of beta globin from the HBB locus produces a beta globin protein comprising the amino acid sequence of SEQ ID NO: 81.

In some embodiments, HDR is mediated by a double-strand break in the HBB gene generated by a site-specific nuclease system. In some embodiments, the site-specific nuclease system comprises a CRISPR nuclease and a single guide RNA capable of hybridizing to the HBB gene. In some embodiments, the single guide RNA capable of hybridizing to the nucleic acid sequence of SEQ ID NO: 27 within the HBB gene.

In another aspect, provided herein in the present disclosure is a recombinant vector comprising a donor polynucleotide described herein. In some embodiments, the vector is selected from the group consisting of viral vectors, plasmids, and ssDNAs. In some embodiments, the recombinant vector is an adeno-associated viral (AAV) vector. In some embodiments, the AAV vector is selected from the group consisting of: AAV1, AAV2, AAV3, AAV4, AAV3, AAV5, AAV6, AAV7, AAV8, AAV9, AAV10, AAV11 and AAV12. In some embodiments, the AAV vector is an AAV6 vector.

In another aspect, provided herein in the present disclosure is a method of expressing exogenous beta globin protein in a cell, the method comprising introducing into the cell: (a) a site-specific nuclease system capable of generating a double-strand break within the HBB gene; and (b) a recombinant vector comprising a HBB donor polynucleotide described herein; whereupon generation of the double-strand break within the HBB gene by the site-specific nuclease system, the nucleic acid sequence of the HBB donor polynucleotide is integrated into the HBB locus by homology directed repair (HDR), resulting in exogenous production of beta globin protein from the HBB locus of the cell. In some embodiments, the method is performed ex vivo.

In some embodiments, the site-specific nuclease system comprises a CRISPR nuclease and a single guide RNA (sgRNA) capable of hybridizing to the HBB gene. In some embodiments, the single guide RNA is capable of hybridizing to the nucleic acid sequence of SEQ ID NO: 27 within the HBB gene. In some embodiments, the CRISPR nuclease is a Cas protein. In some embodiments, the Cas protein is Cas9 or a high-fidelity variant thereof. In some embodiments, the sgRNA and the CRISPR nuclease are incubated together to form a ribonucleoprotein (RNP) complex prior to introducing into the cell. In some embodiments, the RNP complex is introduced into the cell before the recombinant vector. In some embodiments, the sgRNA comprises one or more chemically modified nucleotides. In some embodiments, the modified nucleotide is selected from the group consisting of: a 2′-O-methyl nucleotide, a 2′-O-methyl 3′-phosphorothioate nucleotide, and a 2′-O-methyl 3′-thioPACE nucleotide. In some embodiments, a 5′ end, a 3′ end, or a combination thereof of the modified sgRNA comprises a modified nucleotide.

In some embodiments, the vector is selected from the group consisting of viral vectors, plasmids, and ssDNAs. In some embodiments, the vector is an adeno-associated viral (AAV) vector. In some embodiments, the AAV vector is selected from the group consisting of: AAV1, AAV2, AAV3, AAV4, AAV3, AAV5, AAV6, AAV7, AAV8, AAV9, AAV10, AAV11 and AAV12. In some embodiments, the AAV vector is an AAV6 vector.

In some embodiments, exogenous production of beta globin protein from the HBB locus of the cell is regulated by the native HBB promoter sequence. In some embodiments, the cell is a primary cell. In some embodiments, the primary cell is a mammalian primary cell. In some embodiments, the primary cell is a human cell. In some embodiments, the primary cell is a CD34+ hematopoietic stem and progenitor cell (HSPC). In some embodiments, the HBB gene in the cell comprises one or more genetic mutations associated with a hemoglobinopathy. In some embodiments, the HSPC is isolated from a subject having a hemoglobinopathy resulting from one or more mutations in the HBB gene. In some embodiments, the hemoglobinopathy is sickle cell disease, α-thalassemia, β-thalassemia, or δ-thalassemia. In some embodiments, the hemoglobinopathy is β-thalassemia.

In another aspect, provided herein in the present disclosure is a composition comprising a population of primary hematopoietic stem and progenitor cells (HSPCs) isolated from a subject, wherein one or more primary HSPCs of the population comprise: (a) a site-specific nuclease system capable of generating a double-strand break within the HBB gene; and (b) a recombinant vector comprising the HBB donor polynucleotide described above.

In another aspect, provided herein is a pharmaceutical composition comprising an isolated population of primary hematopoietic stem and progenitor cells (HSPCs) derived from an individual subject having a hemoglobinopathy resulting from one or mutations in the HBB gene, wherein the HSPC population comprises: (a) first plurality of primary HSPCs comprising the one or more mutations in the HBB gene; and (b) a second plurality of primary HSPCs comprising a heterologous polynucleotide integrated into the HBB locus, wherein the heterologous polynucleotide comprises the nucleic acid sequence of a HBB donor polynucleotide described herein. In some embodiments, the population of primary HSPCs is comprised of greater than 10% of the second plurality of primary HSPCs. In some embodiments, the population of primary HSPCs comprises CD34+ HSPCs. In some embodiments, the pharmaceutical composition further comprises a pharmaceutically acceptable carrier. In some embodiments, the individual subject is human.

In another aspect, provided herein is a method for preventing or treating a hemoglobinopathy resulting from one or mutations in the HBB gene in a subject in need thereof, the method comprising administering to the subject a pharmaceutical composition described herein. In some embodiments, the administering comprises autologous transplantation of the pharmaceutical composition to the subject. In other embodiments, the administering comprises allogeneic transplantation of the pharmaceutical composition to the subject. In some embodiments, the subject is a human. In some embodiments, the administering comprises a delivery route selected from the group consisting of intravenous, intraperitoneal, intramuscular, intradermal, subcutaneous, intrathecal, intraosseous, and a combination thereof. In some embodiments, the hemoglobinopathy is sickle cell disease, α-thalassemia, β-thalassemia, or δ-thalassemia. In some embodiments, the hemoglobinopathy is β-thalassemia.

In another aspect, provided herein is an isolated primary HSPC comprising a heterologous polynucleotide integrated into the HBB locus, wherein the heterologous polynucleotide comprises the nucleic acid sequence of a HBB donor polynucleotide described herein.

In another aspect, provided herein is an alpha-1 antitrypsin (AAT) donor polynucleotide comprising, in a 5′ to 3′ orientation: (a) a first Hemoglobin Subunit Alpha 1 (HBA1) homology region comprising a nucleic acid sequence having at least 95% sequence identity to a first target region of the HBA1 gene; (b) an exon 1 region comprising a nucleic acid sequence having at least 95% sequence identity to exon 4 of the alpha-1 antitrypsin (AAT) gene, and which encodes an amino acid sequence encoded by exon 4 of the AAT gene; (c) a heterologous globin intron 1 region comprising a nucleic acid sequence having at least 95% sequence identity to intron 1, or a portion thereof, of a Hemoglobin Subunit gene; (d) an exon 2 region comprising a nucleic acid sequence having at least 95% sequence identity to exon 5 of the AAT gene, and which encodes an amino acid sequence encoded by exon 5 of the AAT gene; (e) a heterologous globin intron 2 region comprising a nucleic acid sequence having at least 95% sequence identity to intron 2, or a portion thereof, of a Hemoglobin Subunit gene; (f) an exon 3 region comprising a nucleic acid sequence having at least 95% sequence identity to exon 6-7 of the AAT gene, and which encodes an amino acid sequence encoded by exon 6-7 of the AAT gene; and (g) a second HBA1 homology region comprising a nucleic acid sequence having at least 95% sequence identity to a second target region of the HBA1 gene, wherein the second target region is positioned 3′ to the first target region in the HBA1 gene; wherein homology directed repair (HDR)-mediated integration of the AAT donor polynucleotide sequence into an HBA1 locus results in exogenous expression of alpha-1 antitrypsin protein from the HBA1 locus.

In some embodiments, the AAT donor polynucleotide comprises a polyadenylation signal sequence positioned between the exon 3 region and the second HBA1 homology region. In some embodiments, the polyadenylation signal sequence is selected from the group consisting of a polyadenylation signal sequence from bovine growth hormone (bGH), human growth hormone (hGH), rabbit beta globin (RbGlob), a synthetic poly A sequence based on rabbit beta globin poly A (SynthRbGlob) and Simian Virus 40 (SV40). In some embodiments, the first target region of the HBA1 gene comprises the nucleic acid sequence of SEQ ID NO: 23. In some embodiments, the second target region of the HBA1 gene comprises the nucleic acid sequence of SEQ ID NO: 24. In some embodiments, the exon 1 region comprises the nucleic acid sequence of SEQ ID NO: 93. In some embodiments, the exon 2 region comprises the nucleic acid sequence of SEQ ID NO: 94. In some embodiments, the exon 3 region comprises the nucleic acid sequence of SEQ ID NO: 95. In some embodiments, the heterologous globin intron 1 region comprises a nucleic acid sequence having at least 95% sequence identity to intron 1, or a portion thereof, of a Hemoglobin Subunit gene selected from the group consisting of Hemoglobin Subunit Alpha 1 (HBA1), Hemoglobin Subunit Beta (HBB), Hemoglobin Subunit Delta (HBD), and Hemoglobin Subunit Gamma 2 (HBG2). In some embodiments, the Hemoglobin Subunit gene is HBA1. In some embodiments, the heterologous globin intron 1 region comprises the nucleic acid sequence of SEQ ID NO: 28. In some embodiments, the heterologous globin intron 2 region comprises a nucleic acid sequence having at least 95% sequence identity to intron 2, or a portion thereof, of a Hemoglobin Subunit gene selected from the group consisting of Hemoglobin Subunit Alpha 1 (HBA1), Hemoglobin Subunit Beta (HBB), Hemoglobin Subunit Delta (HBD), and Hemoglobin Subunit Gamma 2 (HBG2). In some embodiments, the Hemoglobin Subunit gene is HBA1. In some embodiments, the heterologous globin intron 2 region comprises the nucleic acid sequence of SEQ ID NO: 29. In some embodiments, exogenous expression of AAT from the HBA1 locus produces am AAT protein comprising the amino acid sequence of SEQ ID NO: 96.

In some embodiments, HDR is mediated by a double-strand break in the HBA1 gene generated by a site-specific nuclease system. In some embodiments, the site-specific nuclease system comprises a CRISPR nuclease and a single guide RNA capable of hybridizing to the HBA1 gene. In some embodiments, the single guide RNA is capable of hybridizing to the nucleic acid sequence of SEQ ID NO: 25 within the HBA1 gene.

In another aspect, provided herein is a recombinant vector comprising an AAT donor polynucleotide described herein. In some embodiments, the vector is selected from the group consisting of viral vectors, plasmids, and ssDNAs. In some embodiments, the recombinant vector is an adeno-associated viral (AAV) vector. In some embodiments, the AAV vector is selected from the group consisting of: AAV1, AAV2, AAV3, AAV4, AAV3, AAV5, AAV6, AAV7, AAV8, AAV9, AAV10, AAV11 and AAV12. In some embodiments, the AAV vector is an AAV6 vector. \

In another aspect, provided herein is a method of expressing exogenous AAT protein in a cell, the method comprising introducing into the cell: (a) a site-specific nuclease system capable of generating a double-strand break within the HBA1 gene; and (b) a recombinant vector comprising an AAT donor polynucleotide described herein; whereupon generation of the double-strand break within the HBA1 gene by the site-specific nuclease system, the nucleic acid sequence of the AAT donor polynucleotide is integrated into the HBA1 locus by homology directed repair (HDR), resulting in exogenous production of alpha-1 antitrypsin protein from the HBA1 locus of the cell.

In another aspect, provided herein is a composition comprising a population of primary hematopoietic stem and progenitor cells (HSPCs) isolated from a subject, wherein one or more primary HSPCs of the population comprise: (a) a site-specific nuclease system capable of generating a double-strand break within the HBA1 gene; and (b) a recombinant vector comprising an AAT donor polynucleotide described herein.

In another aspect, provided herein is a pharmaceutical composition comprising an isolated population of primary hematopoietic stem and progenitor cells (HSPCs) derived from an individual subject with alpha-1 antitrypsin deficiency, wherein the HSPC population comprises: (a) a first plurality of primary HSPCs comprising the one or more mutations in the AAT gene; and (b) a second plurality of primary HSPCs comprising a heterologous polynucleotide integrated into the HBA1 locus, wherein the heterologous polynucleotide comprises the nucleic acid sequence of an AAT donor polynucleotide described herein. In another aspect, provided herein is a method for preventing or treating alpha-1 antitrypsin deficiency resulting from one or mutations in the AAT gene in a subject in need thereof, the method comprising administering to the subject the pharmaceutical composition described above.

In another aspect, provided herein is an isolated primary HSPC comprising a heterologous polynucleotide integrated into the HBA1 locus, wherein the heterologous polynucleotide comprises the nucleic acid sequence of an AAT donor polynucleotide described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic of T2A-EGFP globin expression reporter system used. Linkage of the T2A-EGFP tag to the 3′end of the inserted gene of interest results in equimolar amounts of protein of interest and EGFP after transcription and translation, which enables the indirect quantification of the protein of interest by measuring the mean fluorescence intensity (MFI) of EGFP.

FIG. 2 shows a schematic of the human α- and β-globin loci on chromosome 16 and 11, respectively. In adulthood, humans mainly express alpha- and beta-globin chains which together form a tetramer of functional hemoglobin (HbA).

FIGS. 3A-3D provide a schematic and results which show that the HBB locus produces higher levels of protein than the HBA1 locus. FIG. 3A provides a schematic of genome editing strategy used to build an endogenous HBB-EGFP control. A T2A-EGFP sequence is knocked into the 3′ end of the endogenous HBB gene by CRISPR-Cas9 genome editing and homologous recombination from a donor template provided in form of AAV6. FIG. 3B provides a schematic of genome editing strategy used by Cromer et al. 2021. A cut is made at the 3′ end of the HBA1 gene and the HBB-T2A-EGFP gene is inserted via homologous recombination. FIG. 3C provides representative flow cytometry results of a HSPCs edited with HBB-EGFP or α-HBB-EGFP, respectively, that have been differentiated into red blood cell progenitors in vitro. Cells were stained with antibodies for CD34, CD45, CD71, and CD235a and gates for CD34−/CD45− and CD71+/CD235a+ cells. Shown are histograms of GFP expression levels. FIG. 3D provides quantification of mean florescence intensities in HBB-EGFP and α-HBB-EGFP cells (n=3). The EGFP MFI for α-HBB-EGFP was normalized to HBB-EGFP in each experiment. Each dot represents a biological replicate.

FIGS. 4A-4C provide a schematic and results which show that introns are necessary for physiological expression of HBB-T2A-EGFP. FIG. 4A shows a fraction of HBB exon 1 showing the alignment of the wild type (top) and diverged HBB coding sequence. Also annotated is the HBB gRNA sequence and respective PAM site. FIG. 4A discloses the protein sequences as SEQ ID NOS 82 and 97, respectively, in order of appearance. FIG. 4B-1-FIG. 4B-3 provide schematics of genome editing strategies for gene replacement of HBB at the HBB locus. Different designs of homology arms and polyA elements were tested. All constructs contain the diverged HBB coding sequence and no introns. FIG. 4C provides flow cytometry results of HSPCs edited with the strategies outlined in B that have been differentiated into red blood cell progenitors in vitro. Cells were stained with antibodies for CD34, CD45, CD71, and CD235a and gated for CD34−/CD45− and CD71+/CD235a+ cells. The EGFP MFIs for β-HBB^{div}-EGFP constructs were normalized to HBB-T2A-EGFP control in each experiment. Each dot represents a biological replicate.

FIGS. 5A-5D provide schematics and results which show that heterologous introns boost HBB-T2A-EGFP expression to physiological levels in CD34-derived RBCs. FIGS. 5A-5C provide schematics of genome editing strategies to insert the HBB gene into the HBB locus. Constructs vary in their design for homology arms, polyA tails and intron sequences. FIG. 5D show flow cytometry results of edited HSPCs that have been differentiated into red blood cell progenitors in vitro. Cells were stained with antibodies for CD34, CD45, CD71, and CD235a and gated for CD34−/CD45− and CD71+/CD235a+ cells. The EGFP MFIs for all constructs were normalized to HBB-EGFP control in each experiment. The dotted line marks endogenous HBB-EGFP expression levels. Each dot represents a biological replicate.

FIGS. 6A-6B provide schematics and results which show that heterologous introns boost HBB expression in CD34-derived RBCs. FIG. 6A provides schematics of genome editing strategies to insert the SCD (E6V) mutation into the HBB gene by using a SNP-donor (left) or whole HBB gene insertion with or without heterologous introns (right). FIG. 6B provides correlation of HDR frequencies with HbS expression from edited HSPCs that have been differentiated into red blood cell progenitors in vitro. HDR frequencies were determined by ddPCR and % HbS protein levels were determined by HPLC analysis. Cells edited with AAV6 donors containing heterologous introns result in similar HbS protein expression per allele to insertion of the SCD mutation using a SNP AAV6 donor. Each dot represents a biological replicate.

FIGS. 7A-7C provide results which show that further optimization of the HBG2-intron AAV6 donor results in higher HBB-T2A-EGFP expression and HDR frequencies. FIG. 7A provides flow cytometry results of HSPC-derived RBCs edited with AAV6 DNA donors containing the HBB diverged coding sequences and HBG2 full length introns with different polyA tails. Cells were differentiated into red blood cell progenitors in vitro, then stained with antibodies for CD34, CD45, CD71, and CD235a and gated for CD34−/CD45− and CD71+/CD235a+ cells. The EGFP MFIs for all constructs were normalized to construct with bGH polyA tail in each experiment (dotted line). Each dot represents a biological replicate. FIG. 7B provide flow cytometry results of HSPC-derived RBCs edited with AAV6 DNA donors containing the HBB diverged coding sequence, HBG2 introns of various lengths and a bGH polyA tail. The EGFP MFIs for all constructs were normalized to construct with full length HBG2 introns in each experiment (dotted line). Each dot represents a biological replicate. FIG. 7C demonstrates truncating HBG2 intron 2 (int2-v2) results in increased knockin efficiency as measured by % EGFP positive HSPC-derived RBCs.

FIGS. 8A-8E provide schematics and results which show that gene editing with AAV6 donors containing heterologous introns rescues the SCD phenotype in RBCs derived from CD34+ HSPCs isolated from SCD patients. FIG. 8A provides a schematic of the AAV6 donor constructs used. All donors contain homology arms to HBB gene, a diverged HBB coding sequence and HBG2 introns. Two different polyA tails were tested (bGH and SV40) and two different lengths of HBG2 intron 2 (i2v2). FIG. 8B provides a schematic of the gene editing procedure. HSPCs from SCD patients were gene-edited with HBB-RNP and AAV6 DNA donors. Edited cells were then differentiated into RBC progenitors in vitro, lysed and protein extracts analyzed by HPLC and RP-HPLC. FIG. 8C provides all HBB gene insertion AAV6 DNA donors tested resulted in high expression of HbA protein comparable to a gene editing approach using a SNP donor that corrects the SCD point mutation. Cleared cell lysates were separated by HPLC and % hemoglobin was determined by integrating area under the peaks (n=6 biological replicates in two different HSPC donors). FIG. 8D provides all HBB gene insertion AAV6 DNA donors tested resulted in a beta to alpha globin chain ratio >0.5. Reverse-phase HPLC results for gene edited SCD HSPC-derived RBCs. Cleared cell lysates were separated by RP-HPLC and beta to alpha chain ratio was determined by integrating area under the peaks. FIG. 8E shows that RBC differentiation potential in vitro is unaffected by gene targeting with AAV6 DNA donors. Flow cytometry results of SCD HSPCs edited with the strategies outlined in A that have been differentiated into red blood cell progenitors in vitro. Cells were stained with antibodies for CD34, CD45, CD71, and CD235a and gated for live, CD34−/CD45− and CD71+/CD235a+ cells (n=8).

FIGS. 9A-9D provide schematics and results which show that addition of heterologous introns enables expression of therapeutic proteins from HBA1 and HBB loci. FIGS. 9A-9B show schematics of gene editing strategies to insert an alpha-1 antitrypsin (AAT) gene to be expressed from HBA1 (FIG. 9A) or HBB (FIG. 9B) gene locus. Two constructs were tested for each approach, one without introns (cDNA only) and one with heterologous globin introns (HBA1 or HBG2, respectively). FIG. 9C shows introns are necessary for high expression of AAT from HBA1 and HBB loci. Representative flow cytometry plots of edited HSPCs that were differentiated into red blood cell progenitors in vitro. Cells were stained with antibodies for CD34, CD45, CD71, and CD235a and gated for CD34−/CD45− and CD71+/CD235a+ cells. AAT expression was measured by either EGFP expression (alpha-globin locus) or by intracellular staining with myc-APC antibody (beta-globin locus). FIG. 9D shows quantification of HSPC-derived RBCs expressing AAT protein measured by either EGFP expression (a-globin) or myc expression (b-globin). Each dot represents a biological replicate.

### DETAILED DESCRIPTION OF THE INVENTION

Hemoglobin disorders are amongst the most common genetic disorders worldwide. Among those, β-thalassemia results in reduced production of β-globin, a protein that forms functional, oxygen-carrying hemoglobin with α-globin (HbA, α₂β₂). Hemoglobin is produced at high levels in red blood cells (RBCs) that circulate from the lungs to all other tissues in the body to deliver oxygen. In the most severe form of the disease, β-thalassemia major, patients present with severe anemia as they carry homozygous or compound heterozygous genetic mutations that completely abolish the production of functional β-globin. Almost 300 different β-thalassemia mutations have been characterized with the vast majority being small nucleotide insertions, substitutions, or deletions within or directly adjacent to the β-globin (HBB) gene. β-thalassemia major and some β-thalassemia intermedia patients typically require lifelong regular blood transfusions combined with iron chelation therapy which carries a substantial clinical and economic burden. Gene replacement therapy has emerged as a potentially viable option for treating β-thalassemia.

Several challenges to gene replacement have presented themselves during gene replacement therapies. Several groups have assessed different strategies to correct aberrant expression of the mutant HBB gene. Currently, the only available cure is an allogeneic hematopoietic stem cell transplant (HSCT) from a matched donor. Often such a donor is not available or if a donor has been found, the risk of immune rejection and graft-vs-host disease remains. Thus, isolating the patient's own hematopoietic stem and progenitor cells (HSPCs) and introducing a functional HBB gene would be an ideal therapeutic strategy as these corrected cells would not be rejected by the patient upon reinfusion. Gene addition using lentiviral vectors stably transfers the HBB gene including introns and regulatory elements to HSPCs and has shown promising outcomes in the clinic. However, lentiviruses integrate semi-randomly which could activate neighboring genes resulting in oncogenesis or clonal expansion. An alternative approach uses CRISPR-Cas9 gene editing to introduce targeted double-strand breaks to transcriptionally upregulate the expression of fetal γ-globin which could compensate for the lack of adult β-globin. While initial results look promising, long-term efficacy of this strategy needs to be determined as it is unclear if high fetal globin expression can be maintained in adult cells where it is normally silenced.

Provided herein, the disclosure provides methods and compositions to introduce a full-length gene to replace an endogenous mutated gene. Methods of treatments and compositions are described herein and are directed to the treatment of β-thalassemia but can be broadly expanded to other diseases or disorders where treatment is amenable with the compositions described herein. The present disclosure describes, inter alia, use of CRISPR-Cas9 to introduce a double stranded break into the mutated HBB gene and introduce a donor polynucleotide comprising the HBB gene lacking disease-causing mutations. The HBB gene lacking mutations replaces the mutated gene through homology-directed recombination (HDR) through homology arms flanking the gene present in the donor polynucleotide. In this present disclosure, the strategy provides an HBB sequence in the donor polynucleotide sequence that is not identical to the wild-type HBB nucleotide sequence to promote HDR through the homology arms instead of through homology within the gene. Furthermore, the strategy provides methods to maintain endogenous regulatory mechanisms by inclusion of introns of HBB or related hemoglobin genes.

### Definitions

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

Generally, nomenclatures used in connection with, and techniques of, cell and tissue culture, molecular biology, immunology, microbiology, genetics and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. The promoter can be a heterologous promoter.

As used herein, the terms “subject”, “individual” or “patient” refer, interchangeably, to a warm-blooded animal such as a mammal. In particular embodiments, the term refers to a human. A subject may have, be suspected of having, or be predisposed to, for example a hemoglobinopathy or other disease described herein. The term also includes livestock, pet animals, or animals kept for study, including horses, cows, sheep, poultry, pigs, cats, dogs, zoo animals, goats, primates (e.g. chimpanzee), and rodents. A “subject in need thereof” refers to a subject that has one or more symptoms of, for example, beta thalassemia, that has received a diagnosis, or that is suspected of having or being predisposed to beta thalassemia, that shows a deficiency of functional beta globin or a polypeptide encoded by HBB as described herein, or that is thought to potentially benefit from increased expression of functional beta globin as described herein.

The term “administering” as used herein refers to a method of giving a dosage of a composition (e.g., a cell therapy composition) to a subject. The method of administration can vary depending on various factors (e.g., the pharmaceutical composition being administered, and the severity of the condition, disease, or disorder being treated).

The term “treating” or “treatment” refers to any one of the following: ameliorating one or more symptoms of a disease or condition (e.g., beta thalassemia); preventing the manifestation of such symptoms before they occur; slowing down or completely preventing the progression of the disease or condition (as may be evident by longer periods between reoccurrence episodes, slowing down or prevention of the deterioration of symptoms, etc.); enhancing the onset of a remission period; slowing down the irreversible damage caused in the progressive-chronic stage of the disease or condition (both in the primary and secondary stages); delaying the onset of said progressive stage; or any combination thereof.

The terms “about” and “approximately” mean within 20%, within 15%, within 10%, within 9%, within 8%, within 7%, within 6%, within 5%, within 4%, within 3%, within 2%, within 1%, or less of a given value or range.

The term “identity,” or “homology” as used interchangeable herein, may be to calculations of “identity,” “homology,” or “percent homology” between two or more nucleotide or amino acid sequences that can be determined by aligning the sequences for optimal comparison purposes (e.g., gaps can be introduced in the sequence of a first sequence). The nucleotides at corresponding positions may then be compared, and the percent identity between the two sequences may be a function of the number of identical positions shared by the sequences (i.e., % homology=#of identical positions/total #of positions×100). For example, a position in the first sequence may be occupied by the same nucleotide as the corresponding position in the second sequence, then the molecules are identical at that position. The percent homology between the two sequences may be a function of the number of identical positions shared by the sequences, taking into account the number of gaps, and the length of each gap, which need to be introduced for optimal alignment of the two sequences. In some embodiments, the length of a sequence aligned for comparison purposes may be at least about: 30%, 40%, 50%, 60%, 65%, 70%, 75%, 80%, 85%, 90%, 91%, 92%, 93%, 94%, 95%, 96%, 97%, 98%, or 95%, of the length of the reference sequence. A BLAST® search may determine homology between two sequences. The two sequences can be genes, nucleotides sequences, protein sequences, peptide sequences, amino acid sequences, or fragments thereof. The actual comparison of the two sequences can be accomplished by well-known methods, for example, using a mathematical algorithm. A non-limiting example of such a mathematical algorithm may be described in Karlin, S. and Altschul, S., Proc. Natl. Acad. Sci. USA, 90-5873-5877 (1993). Such an algorithm may be incorporated into the NBLAST and XBLAST programs (version 2.0), as described in Altschul, S. et al., Nucleic Acids Res., 25:3389-3402 (1997). When utilizing BLAST and Gapped BLAST programs, any relevant parameters of the respective programs (e.g., NBLAST) can be used. For example, parameters for sequence comparison can be set at score=100, word length=12, or can be varied (e.g., W=5 or W=20). Other examples include the algorithm of Myers and Miller, CABIOS (1989), ADVANCE, ADAM, BLAT, and FASTA. In another embodiment, the percent identity between two amino acid sequences can be accomplished using, for example, the GAP program in the GCG software package (Accelrys, Cambridge, UK).

By “donor polynucleotide,” the present disclosure refers to a polynucleotide sequence comprising a gene sequence (including, for example, coding and non-coding regulatory sequences) that is flanked by a 5′ and 3′ homology arm that is complementary to the gene that is to be replaced. The donor polynucleotide can be a circular plasmid, linear, or made to be linear through a cleavage process.

A “Cas molecule,” as used herein, refers to a Cas polypeptide or a nucleic acid encoding a Cas9 polypeptide. A “Cas polypeptide” is a polypeptide that can interact with a gRNA molecule and, in concert with the gRNA molecule, localize to a site comprising a target domain and, in certain embodiments, a PAM sequence. Cas molecules include both naturally occurring Cas molecules and Cas molecules and engineered, altered, or modified Cas molecules or Cas polypeptides that differ, e.g., by at least one amino acid residue, from a reference sequence, e.g., the most similar naturally occurring Cas molecule. (The terms altered, engineered or modified, as used in this context, refer merely to a difference from a reference or naturally occurring sequence, and impose no specific process or origin limitations.) A Cas molecule may be a Cas9 polypeptide or a nucleic acid encoding a Cas9 polypeptide. A Cas molecule may be a nuclease (an enzyme that cleaves both strands of a double-stranded nucleic acid), a nickase (an enzyme that cleaves one strand of a double-stranded nucleic acid), or an enzymatically inactive (or dead) Cas molecule. Exemplary Cas molecules include high-fidelity Cas variants having improved on-target specificity and reduced off-target activity. Examples of high-fidelity Cas9 variants include but are not limited to those described in PCT Publication Nos. WO/2018/068053 and WO/2019/074542, each of which is herein incorporated by reference in its entirety.

As used herein, the term “gRNA molecule” or “gRNA” refers to a guide RNA which is capable of targeting a Cas molecule to a target nucleic acid. In one embodiment, the term “gRNA molecule” refers to a guide ribonucleic acid. In another embodiment, the term “gRNA molecule” refers to a nucleic acid encoding a gRNA. In one embodiment, a gRNA molecule is non-naturally occurring. In one embodiment, a gRNA molecule is a synthetic gRNA molecule.

“HDR”, or “homology-directed repair,” as used herein, refers to the process of repairing DNA damage using a homologous nucleic acid (e.g., an endogenous homologous sequence, e.g., a sister chromatid, or an exogenous nucleic acid, e.g., a template nucleic acid such as a donor polynucleotide described herein). Canonical HDR typically acts when there has been significant resection at the double strand break, forming at least one single stranded portion of DNA. In a normal cell, HDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2, and the homologous nucleic acid is typically double-stranded. This process is used by a number of site-specific nuclease systems that create a double-strand break, such as meganucleases, zinc finger nucleases (ZFNs), transcription activator-like effector nucleases (TALENs), and the CRISPR-Cas gene editing systems. In particular embodiments, HDR involves double-stranded breaks induced by CRISPR-Cas nuclease, e.g. Cas9.

As used herein, “functional” in the context of a protein product (or coding sequences thereof) refers to a protein of interest (and its related coding sequences) having similar or equivalent protein function as its wild-type counterpart, for example, wild type beta globin protein (UniProtKB-O95408), which is referred to herein as “functional beta globin protein.” In some embodiments, functional beta globin protein has at least 50%, 55%, 60%, 65%, 70%, 75%, 80%, 85%, 90%, 95%, 97%, 99%, 99.5%, 99.7%, 99.9% or 100% of the function of wild-type beta globin protein, as determined by any method known in the art for assessing beta globin protein function.

As used herein, “heterologous” in the context of an intron sequence means that the intron sequence (or portion thereof) is not naturally associated with its linked coding sequence within the donor polynucleotide. For example, when a heterologous intron is said to be operably linked to a coding sequence within a donor polynucleotide described herein, it means that the heterologous intron is derived from one gene whereas the coding sequence is derived from another, different gene. In some embodiments, a heterologous intron is derived from a gene locus that is also different from the gene locus being targeted by the donor polynucleotide in which its contained. In other embodiments, a heterologous intron is derived from same gene locus as the gene locus being targeted by its donor polynucleotide.

### Targeted Gene Insertion

The present disclosure provides compositions and methods for introducing a portion of an exogenous polynucleotide sequence into a target site of an endogenous polynucleotide sequence at a gene locus where the polynucleotide sequence may comprise at least one mutation. The mutation can cause aberrant expression and can manifest as a disease pathology such as but is not limited to beta-thalassemia. One such strategy and method to fix or ameliorate aberrant expression caused by a mutation associated with a disease state is described herein.

CRISPR-Cas systems are quickly emerging as an attractive tool to introduce double stranded breaks. Briefly, CRISPR-Cas systems utilize a guide RNA or guide polynucleotide to guide the Cas nuclease to a target site to introduce a double stranded break into the sequence.

A donor template or donor polynucleotide sequence can be used simultaneously to utilize HDR machinery that can resect the donor polynucleotide sequence into the endogenous sequence through the regions of the donor polynucleotide having high homology or sequence identity. In this manner, targeted gene insertion can be performed by administering a site-specific nuclease system in combination with a donor polynucleotide.

In embodiments, the donor polynucleotide comprises an exogenous sequence (including coding and non-coding regulatory sequences) that is flanked by regions containing high homology with the endogenous targeted locus. In some embodiments, the targeted gene insertion can replace at least a portion of the endogenous polynucleotide sequence. In particular embodiments, the exogenous sequence is integrated into the translational start site of the targeted gene locus. In particular embodiments, the exogenous sequence that is integrated into the host cell genome is expressed under control of the native promoter sequence of the targeted gene locus.

Endogenous polynucleotides may contain polymorphisms or mutations that cause expression of an aberrant protein that results in the manifestation of a disease, such as beta-thalassemia. In some embodiments, the endogenous polynucleotide sequence comprises mutations, including but are not limited to missense and non-sense mutations. In some embodiments, the endogenous polynucleotide sequence can comprise insertions, deletions, or truncations.

### Donor Polynucleotide

Diverged Exon Sequences

The donor polynucleotide can comprise an exogenous polynucleotide sequence that replaces an endogenous sequence within a gene locus in a cell. In instances where the targeted gene within the cell harbors one or more mutations, and correction or replacement of the mutant allele within its native locus is desired, the donor polynucleotide can comprise an exogenous polynucleotide sequence encoding a wild-type functional copy of the targeted gene, including intronic sequences to facilitate its expression. Ideally, HDR of the exogenous polynucleotide occurs only through the 5′ and 3′ homology arms that flank the donor gene, so that the entirety of the exogenous polynucleotide sequence between the homology arms is integrated into the targeted locus. However, where the donor gene shares high nucleotide sequence identity with the targeted mutant allele, undesired partial recombination events can lead to incomplete or unsuccessful integration of the entirety of the intended donor sequence. To avoid these outcomes, the exogenous polynucleotide sequence may be diverged between the homology arms to reduce the percent identity between the donor gene and the endogenous gene to be replaced, while still encoding for functional protein.

Because of the degeneracy of the genetic code, a large number of functionally identical nucleic acid sequences can encode any given protein. For instance, the codons GCA, GCC, GCG and GCU all encode the amino acid alanine. Thus, at every position where an alanine is specified by a codon, the codon can be diverged to any of its corresponding alternative codons without altering the encoded polypeptide. Such nucleic acid variations are “silent variations,” and every nucleic acid sequence described herein that encodes a polypeptide also describes every possible silent variation of the nucleic acid encoding that polypeptide. One of skill will recognize that each codon in a nucleic acid (except AUG, which is ordinarily the only codon for methionine, and TGG, which is ordinarily the only codon for tryptophan) can be modified (diverged) to yield a functionally identical polypeptide. Alternate codons for each amino acid are provided in Table 1 below.

TABLE 1
Codon Table

| One | Three | | |
|---|---|---|---|
| letter | letter | | |
| code | code | Amino acid | Possible codons |
| A | Ala | Alanine | GCA, GCC, GCG, GCT |
| C | Cys | Cysteine | TGC, TGT |
| D | Asp | Aspartic acid | GAC, GAT |
| E | Glu | Glutamic acid | GAA, GAG |
| F | Phe | Phenylalanine | TTC, TTT |
| G | Gly | Glycine | GGA, GGC, GGG GGT |
| H | His | Histidine | CAC, CAT |
| I | Ile | Isoleucine | ATA, ATC, ATT |
| K | Lys | Lysine | AAA, AAG |
| L | Leu | Leucine | CTA, CTC, CTG, CTT, TTA, TTG |
| M | Met | Methionine | ATG |
| N | Asn | Asparagine | AAC, AAT |
| P | Pro | Proline | CCA, CCC, CCG, CCT |
| Q | Gln | Glutamine | CAA, CAG |
| R | Arg | Arginine | AGA, AGG, CGA, CGC, CGG, CGT |
| S | Ser | Serine | AGC, AGT, TCA, TCC, TCG, TCT |
| T | Thr | Threonine | ACA, ACC, ACG, ACT |
| V | Val | Valine | GTA, GTC, GTG, GTT |
| W | Trp | Tryptophan | TGG |
| Y | Tyr | Tyrosine | TAC, TAT |
| * | * | stop codon | TAA, TAG, TGA |

Serine and Arginine can be diverged by up to 100%; Leucine and stop codons can be diverged by up to 66%; and Alanine, Cysteine, Aspartic Acid, Glutamic Acid, Phenylalanine, Glycine, Histidine, Isoleucine, Lysine, Asparagine, Proline, Glutamine, Threonine, Valine, Tyrosine can be diverged by 33%. Accordingly, for any desired protein to be expressed from a donor polynucleotide described herein, a diverged coding sequence can be devised based on alternate codons available for each amino acid position, up to a maximally diverged nucleotide sequence. Further consideration can be given to the frequency of a particular codon's usage in a particular species, tissue and/or cell type (see e.g., Plotkin et al., PNAS 101(34):12588-12591 (2004)), to optimize expression of the protein while maintaining sufficient nucleotide divergence from the target gene. Where exons are positioned within a donor polynucleotide with intervening heterologous introns, the coding sequences of the donor polynucleotide can be diverged on an exon-by-exon basis, even where heterologous introns maintain high sequence identity to its native sequence, to sufficiently decrease the overall homology between the donor polynucleotide sequence and that of the targeted gene, other than with respect to the homology arms which necessarily share high sequence identity to effect successful integration of the complete donor polynucleotide sequence.

Sequence divergence strategies provided herein also contemplate use of “conservatively modified variants” which applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. With regard to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. In some cases, conservatively modified variants of a protein can have an increased stability, assembly, or activity as described herein.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
   (see, e.g., Creighton, Proteins, W. H. Freeman and Co., N. Y. (1984)).

In some embodiments, the percent nucleotide identity between the exogenous donor polynucleotide sequence (other than the homology arms) and endogenous polynucleotide sequence to be replaced (i.e. gene target) is no more than 95%, while encoding the same amino acid sequence. In some embodiments, the percent identity between the exogenous polynucleotide sequence and endogenous polynucleotide sequence to be replaced is about 60% to about 95% while encoding the same amino acid sequence. In some embodiments, the percent identity between the exogenous polynucleotide sequence and endogenous polynucleotide sequence to be replaced is about 60% to about 65%, about 60% to about 70%, about 60% to about 75%, about 60% to about 80%, about 60% to about 85%, about 60% to about 90%, about 60% to about 95%, about 60% to about 97%, about 60% to about 98%, about 60% to about 99%, about 65% to about 70%, about 65% to about 75%, about 65% to about 80%, about 65% to about 85%, about 65% to about 90%, about 65% to about 95%, about 65% to about 97%, about 65% to about 98%, about 65% to about 99%, about 70% to about 75%, about 70% to about 80%, about 70% to about 85%, about 70% to about 90%, about 70% to about 95%, about 70% to about 97%, about 70% to about 98%, about 70% to about 99%, about 75% to about 80%, about 75% to about 85%, about 75% to about 90%, about 75% to about 95%, about 75% to about 97%, about 75% to about 98%, about 75% to about 99%, about 80% to about 85%, about 80% to about 90%, about 80% to about 95%, about 80% to about 97%, about 80% to about 98%, about 80% to about 99%, about 85% to about 90%, about 85% to about 95%, about 85% to about 97%, about 85% to about 98%, about 85% to about 99%, about 90% to about 95%, about 90% to about 97%, about 90% to about 98%, about 90% to about 99%, about 95% to about 97%, about 95% to about 98%, about 95% to about 99%, about 97% to about 98%, about 97% to about 99%, or about 98% to about 99% while encoding the same amino acid sequence.

The donor polynucleotide can comprise an exogenous polynucleotide sequence comprising a coding sequence of HBB. In some embodiments, and in accordance with the divergence strategies described above, the transgene of the exogenous polynucleotide sequence and the target gene locus are not identical in sequence. In some embodiments, the percent identity between the HBB coding sequence of the donor polynucleotide and the HBB allele to be replaced is about 60% to about 95%. In some embodiments, the percent identity between the HBB coding sequence of the donor polynucleotide and the HBB allele to be replaced is about 60% to about 65%, about 60% to about 70%, about 60% to about 75%, about 60% to about 80%, about 60% to about 85%, about 60% to about 90%, about 60% to about 95%, about 60% to about 97%, about 60% to about 98%, about 60% to about 99%, about 65% to about 70%, about 65% to about 75%, about 65% to about 80%, about 65% to about 85%, about 65% to about 90%, about 65% to about 95%, about 65% to about 97%, about 65% to about 98%, about 65% to about 99%, about 70% to about 75%, about 70% to about 80%, about 70% to about 85%, about 70% to about 90%, about 70% to about 95%, about 70% to about 97%, about 70% to about 98%, about 70% to about 99%, about 75% to about 80%, about 75% to about 85%, about 75% to about 90%, about 75% to about 95%, about 75% to about 97%, about 75% to about 98%, about 75% to about 99%, about 80% to about 85%, about 80% to about 90%, about 80% to about 95%, about 80% to about 97%, about 80% to about 98%, about 80% to about 99%, about 85% to about 90%, about 85% to about 95%, about 85% to about 97%, about 85% to about 98%, about 85% to about 99%, about 90% to about 95%, about 90% to about 97%, about 90% to about 98%, about 90% to about 99%, about 95% to about 97%, about 95% to about 98%, about 95% to about 99%, about 97% to about 98%, about 97% to about 99%, or about 98% to about 99%. In some embodiments, the percent identity between the HBB coding sequence of the donor polynucleotide and the wild-type HBB cDNA sequence (SEQ ID NO: 7) is about 60% to about 95%.

In some embodiments, the donor polynucleotide comprises at least 70% sequence identity to SEQ ID NO: 1—SEQ ID NO: 5. In some embodiments, the donor polynucleotide comprises at least 70% sequence identity to SEQ ID NO: 88—SEQ ID NO: 91. In other embodiments, the donor polynucleotide comprises at least 70% sequence identity to SEQ ID NOL 72—SEQ ID NO: 73.

Heterologous Introns

Known strategies to introduce a coding sequence into a donor polynucleotide include use of a complementary DNA (cDNA) sequence that lack introns. However, as demonstrated in the Examples provided below, inclusion of introns into a donor polynucleotide can increase exogenous protein levels following knock-in, as introns may utilize regulatory mechanisms that can improve overall expression of the donor gene, compared to a cDNA sequence lacking introns but encoding for the same protein. In some embodiments, the included heterologous introns maintain the genomic structure of the endogenous gene being targeted. For example, HBB in its genomic locus context is arranged in the following manner: Exon 1-Intron 1-Exon 2-Intron 2-Exon 3. In some embodiments, intron 1 of a related globin gene (non-HBB) can be positioned 3′ to exon 1 of the transgene (for example, a correct copy of HBB) in the donor polynucleotide to maintain appropriate splicing intermediates, and a heterologous intron 2 can be similarly positioned 3′ to exon 2 of the transgene. In some embodiments the heterologous introns comprise sequences derived from hemoglobin genes of a different species, such as monkeys or other mammals. In some embodiments, the related globin gene from which the heterologous intron(s) sequences are derived is selected from the group consisting of Hemoglobin Subunit Alpha 1 (HBA1) gene, Hemoglobin Subunit Beta (HBB), Hemoglobin Subunit Delta (HBD), and Hemoglobin Subunit Gamma 2 (HBG2).

Without being bound by theory, this strategy can be expanded to other genes beyond HBB. Current strategies that utilize targeted gene insertion remove the introns, leaving only the exons encoding the protein of interest. The present disclosure describes inclusion of introns, heterologous introns, or introns of sufficient sequence divergence to decrease the sequence identity of the exogenous polynucleotide sequence flanked by the 5′ and 3′ homology arms.

In some embodiments, inclusion of at least one intron into the donor polynucleotide can increase expression of the gene present in the donor polynucleotide by at least 30% compared to a sequence lacking introns. In some embodiments, inclusion of at least one intron into the can increase expression of the gene present in the donor polynucleotide by at least about 30% to about 99% compared to a sequence lacking introns. In some embodiments, inclusion of at least one intron into the donor polynucleotide can increase expression of the gene present in the donor polynucleotide by at least at least about 30% compared to a sequence lacking introns. In some embodiments, inclusion of at least one intron into the donor polynucleotide can increase expression of the gene present in the donor polynucleotide by at least at most about 99% compared to a sequence lacking introns. In some embodiments, inclusion of at least one intron into the donor polynucleotide can increase expression of the gene present in the donor polynucleotide by at least about 30% to about 40%, about 30% to about 50%, about 30% to about 60%, about 30% to about 65%, about 30% to about 70%, about 30% to about 75%, about 30% to about 80%, about 30% to about 85%, about 30% to about 90%, about 30% to about 95%, about 30% to about 99%, about 40% to about 50%, about 40% to about 60%, about 40% to about 65%, about 40% to about 70%, about 40% to about 75%, about 40% to about 80%, about 40% to about 85%, about 40% to about 90%, about 40% to about 95%, about 40% to about 99%, about 50% to about 60%, about 50% to about 65%, about 50% to about 70%, about 50% to about 75%, about 50% to about 80%, about 50% to about 85%, about 50% to about 90%, about 50% to about 95%, about 50% to about 99%, about 60% to about 65%, about 60% to about 70%, about 60% to about 75%, about 60% to about 80%, about 60% to about 85%, about 60% to about 90%, about 60% to about 95%, about 60% to about 99%, about 65% to about 70%, about 65% to about 75%, about 65% to about 80%, about 65% to about 85%, about 65% to about 90%, about 65% to about 95%, about 65% to about 99%, about 70% to about 75%, about 70% to about 80%, about 70% to about 85%, about 70% to about 90%, about 70% to about 95%, about 70% to about 99%, about 75% to about 80%, about 75% to about 85%, about 75% to about 90%, about 75% to about 95%, about 75% to about 99%, about 80% to about 85%, about 80% to about 90%, about 80% to about 95%, about 80% to about 99%, about 85% to about 90%, about 85% to about 95%, about 85% to about 99%, about 90% to about 95%, about 90% to about 99%, or about 95% to about 99% compared to a sequence lacking introns.

The donor polynucleotide can comprise an exogenous polynucleotide sequence comprising more than 1 heterologous intron. In some embodiments, the exogenous polynucleotide sequence can comprise about 1 heterologous intron to about 10 heterologous introns. In some embodiments, the exogenous polynucleotide sequence can comprise about 1 heterologous intron to about 2 heterologous introns, about 1 heterologous intron to about 3 heterologous introns, about 1 heterologous intron to about 4 heterologous introns, about 1 heterologous intron to about 5 heterologous introns, about 1 heterologous intron to about 6 heterologous introns, about 1 heterologous intron to about 7 heterologous introns, about 1 heterologous intron to about 8 heterologous introns, about 1 heterologous intron to about 9 heterologous introns, about 1 heterologous intron to about 10 heterologous introns; about 2 heterologous introns to about 3 heterologous introns, about 2 heterologous introns to about 4 heterologous introns, about 2 heterologous introns to about 5 heterologous introns, about 2 heterologous introns to about 6 heterologous introns, about 2 heterologous introns to about 7 heterologous introns, about 2 heterologous introns to about 8 heterologous introns, about 2 heterologous introns to about 9 heterologous introns, about 2 heterologous introns to about 10 heterologous introns; about 3 heterologous introns to about 4 heterologous introns, about 3 heterologous introns to about 5 heterologous introns, about 3 heterologous introns to about 6 heterologous introns, about 3 heterologous introns to about 7 heterologous introns, about 3 heterologous introns to about 8 heterologous introns, about 3 heterologous introns to about 9 heterologous introns, about 3 heterologous introns to about 10 heterologous introns; about 4 heterologous introns to about 5 heterologous introns, about 4 heterologous introns to about 6 heterologous introns, about 4 heterologous introns to about 7 heterologous introns, about 4 heterologous introns to about 8 heterologous introns, about 4 heterologous introns to about 9 heterologous introns, about 4 heterologous introns to about 10 heterologous introns; about 5 heterologous introns to about 6 heterologous introns, about 5 heterologous introns to about 7 heterologous introns, about 5 heterologous introns to about 8 heterologous introns, about 5 heterologous introns to about 9 heterologous introns, about 5 heterologous introns to about 10 heterologous introns; about 6 heterologous introns to about 7 heterologous introns, about 6 heterologous introns to about 8 heterologous introns, about 6 heterologous introns to about 9 heterologous introns, about 6 heterologous introns to about 10 heterologous introns; about 7 heterologous introns to about 8 heterologous introns, about 7 heterologous introns to about 9 introns, about 7 introns to about 10 introns; about 8 introns to about 9 introns, about 8 introns to about 10 introns.

In some embodiments, the non-coding sequences comprise no more than 90% sequence identity to the intron of a targeted gene. For example, the donor polynucleotide can comprise the coding sequence for HBB, and further comprise an intron wherein the intron comprises only at most 90% sequence identity to the endogenous HBB intron or SEQ ID NO 9 or SEQ ID NO 10.

In some embodiments, the heterologous intron comprises an intron selection from the group consisting of HBA1, HBG2, HBD, introns from non-human primates, scrambled intron sequences, and engineered intron sequences. In some embodiments, the heterologous intron sequence comprises modifications (e.g. deletions or truncations) that minimize the size of the intron and the overall donor polynucleotide, which can improve HDR rates, while maintaining or improving upon expression of the transgene relative to its endogenous counterpart gene (as demonstrated in Example 5 below). In some embodiments, the modified heterologous intron is derived from intron 2 of the HBG gene. In some embodiments, the modification to intron 2 of HBG2 is deletion of nucleotides 21-437 and 513-834 from the wild-type HBG2 intron 2 sequence (SEQ ID NO: 78).

In some embodiments, the heterologous intron can comprise a sequence derived from HBB intron 1 (SEQ ID NO: 9), HBB intron 2 (SEQ ID NO: 10), HBG2 intron 1 (SEQ ID NO: 11), HBG2 intron 2 (SEQ ID NO: 12), HBD intron 1 (SEQ ID NO: 13), HBD intron 2 (SEQ ID NO: 14), a monkey-derived intron comprising the sequence of SEQ ID NO: 15 or SEQ ID NO: 16. In some embodiments, the heterologous intron can comprise at least 70% sequence identity to an intron sequence selected from the group consisting of SEQ ID NO 9—SEQ ID NO 16 and SEQ ID NO: 78. In some embodiments, the heterologous intron can comprise about 70% sequence identity to about 99% sequence identity to an intron sequence selected from the group consisting of SEQ ID NO 9—SEQ ID NO 16 and SEQ ID NO: 78. In some embodiments, the heterologous intron can comprise about 70% sequence identity to about 75% sequence identity, about 70% sequence identity to about 80% sequence identity, about 70% sequence identity to about 85% sequence identity, about 70% sequence identity to about 90% sequence identity, about 70% sequence identity to about 95% sequence identity, about 70% sequence identity to about 97% sequence identity, about 70% sequence identity to about 98% sequence identity, about 70% sequence identity to about 99% sequence identity, about 75% sequence identity to about 80% sequence identity, about 75% sequence identity to about 85% sequence identity, about 75% sequence identity to about 90% sequence identity, about 75% sequence identity to about 95% sequence identity, about 75% sequence identity to about 97% sequence identity, about 75% sequence identity to about 98% sequence identity, about 75% sequence identity to about 99% sequence identity, about 80% sequence identity to about 85% sequence identity, about 80% sequence identity to about 90% sequence identity, about 80% sequence identity to about 95% sequence identity, about 80% sequence identity to about 97% sequence identity, about 80% sequence identity to about 98% sequence identity, about 80% sequence identity to about 99% sequence identity, about 85% sequence identity to about 90% sequence identity, about 85% sequence identity to about 95% sequence identity, about 85% sequence identity to about 97% sequence identity, about 85% sequence identity to about 98% sequence identity, about 85% sequence identity to about 99% sequence identity, about 90% sequence identity to about 95% sequence identity, about 90% sequence identity to about 97% sequence identity, about 90% sequence identity to about 98% sequence identity, about 90% sequence identity to about 99% sequence identity, about 95% sequence identity to about 97% sequence identity, about 95% sequence identity to about 98% sequence identity, about 95% sequence identity to about 99% sequence identity, about 97% sequence identity to about 98% sequence identity, about 97% sequence identity to about 99% sequence identity, or about 98% sequence identity to about 99% sequence identity to an intron sequence selected from the group consisting of SEQ ID NO: 9—SEQ ID NO: 16 and SEQ ID NO: 78. Is some embodiments, the intron sequence is selected from the group consisting of SEQ ID NO: 9—SEQ ID NO: 16 and SEQ ID NO: 78.

Homology Arms

In preferred embodiments, the 5′ and 3′ homology arms of the donor polynucleotide have at least 95% sequence identity, respectively, with a distinct region of the target gene locus, so that HDR of the exogenous polynucleotide occurs only through the 5′ and 3′ homology arms, and the entirety of the exogenous polynucleotide sequence between the homology arms is integrated into the targeted locus. In some embodiments, the homology arms comprise sequences that target integration of the donor polynucleotide just downstream of the native promoter of the target gene, such that the integrated donor sequence is transcribed from and regulated by the native promoter sequence of the targeted gene. In other embodiments, the homology arms comprise sequences that target integration of the donor polynucleotide into the gene locus such that the target gene is replaced in whole or in part, for example, only with respect to regions of the target gene that harbor mutations. In some such embodiments, the target gene promoter is left intact in order to regulate expression of the transgene.

The homology arms can be of variable lengths. In some embodiments, the 5′ and 3′ homology arms can be identical in length. In some embodiments the 5′ and 3′ homology arms can be different lengths.

In some embodiments, the 5′ homology arm comprises about 50 base pairs to about 1,000 base pairs. In some embodiments, the 5′ homology arm comprises at least about 50 base pairs. In some embodiments, the 5′ homology arm comprises at most about 1,000 base pairs. In some embodiments, the 5′ homology arm comprises about 50 base pairs to about 100 base pairs, about 50 base pairs to about 150 base pairs, about 50 base pairs to about 200 base pairs, about 50 base pairs to about 250 base pairs, about 50 base pairs to about 300 base pairs, about 50 base pairs to about 350 base pairs, about 50 base pairs to about 400 base pairs, about 50 base pairs to about 450 base pairs, about 50 base pairs to about 500 base pairs, about 50 base pairs to about 750 base pairs, about 50 base pairs to about 1,000 base pairs, about 100 base pairs to about 150 base pairs, about 100 base pairs to about 200 base pairs, about 100 base pairs to about 250 base pairs, about 100 base pairs to about 300 base pairs, about 100 base pairs to about 350 base pairs, about 100 base pairs to about 400 base pairs, about 100 base pairs to about 450 base pairs, about 100 base pairs to about 500 base pairs, about 100 base pairs to about 750 base pairs, about 100 base pairs to about 1,000 base pairs, about 150 base pairs to about 200 base pairs, about 150 base pairs to about 250 base pairs, about 150 base pairs to about 300 base pairs, about 150 base pairs to about 350 base pairs, about 150 base pairs to about 400 base pairs, about 150 base pairs to about 450 base pairs, about 150 base pairs to about 500 base pairs, about 150 base pairs to about 750 base pairs, about 150 base pairs to about 1,000 base pairs, about 200 base pairs to about 250 base pairs, about 200 base pairs to about 300 base pairs, about 200 base pairs to about 350 base pairs, about 200 base pairs to about 400 base pairs, about 200 base pairs to about 450 base pairs, about 200 base pairs to about 500 base pairs, about 200 base pairs to about 750 base pairs, about 200 base pairs to about 1,000 base pairs, about 250 base pairs to about 300 base pairs, about 250 base pairs to about 350 base pairs, about 250 base pairs to about 400 base pairs, about 250 base pairs to about 450 base pairs, about 250 base pairs to about 500 base pairs, about 250 base pairs to about 750 base pairs, about 250 base pairs to about 1,000 base pairs, about 300 base pairs to about 350 base pairs, about 300 base pairs to about 400 base pairs, about 300 base pairs to about 450 base pairs, about 300 base pairs to about 500 base pairs, about 300 base pairs to about 750 base pairs, about 300 base pairs to about 1,000 base pairs, about 350 base pairs to about 400 base pairs, about 350 base pairs to about 450 base pairs, about 350 base pairs to about 500 base pairs, about 350 base pairs to about 750 base pairs, about 350 base pairs to about 1,000 base pairs, about 400 base pairs to about 450 base pairs, about 400 base pairs to about 500 base pairs, about 400 base pairs to about 750 base pairs, about 400 base pairs to about 1,000 base pairs, about 450 base pairs to about 500 base pairs, about 450 base pairs to about 750 base pairs, about 450 base pairs to about 1,000 base pairs, about 500 base pairs to about 750 base pairs, about 500 base pairs to about 1,000 base pairs, or about 750 base pairs to about 1,000 base pairs.

In some embodiments, the 3′ homology arm comprises about 50 base pairs to about 1,000 base pairs. In some embodiments, the 3′ homology arm comprises at least about 50 base pairs. In some embodiments, the 3′ homology arm comprises at most about 1,000 base pairs. In some embodiments, the 3′ homology arm comprises about 50 base pairs to about 100 base pairs, about 50 base pairs to about 150 base pairs, about 50 base pairs to about 200 base pairs, about 50 base pairs to about 250 base pairs, about 50 base pairs to about 300 base pairs, about 50 base pairs to about 350 base pairs, about 50 base pairs to about 400 base pairs, about 50 base pairs to about 450 base pairs, about 50 base pairs to about 500 base pairs, about 50 base pairs to about 750 base pairs, about 50 base pairs to about 1,000 base pairs, about 100 base pairs to about 150 base pairs, about 100 base pairs to about 200 base pairs, about 100 base pairs to about 250 base pairs, about 100 base pairs to about 300 base pairs, about 100 base pairs to about 350 base pairs, about 100 base pairs to about 400 base pairs, about 100 base pairs to about 450 base pairs, about 100 base pairs to about 500 base pairs, about 100 base pairs to about 750 base pairs, about 100 base pairs to about 1,000 base pairs, about 150 base pairs to about 200 base pairs, about 150 base pairs to about 250 base pairs, about 150 base pairs to about 300 base pairs, about 150 base pairs to about 350 base pairs, about 150 base pairs to about 400 base pairs, about 150 base pairs to about 450 base pairs, about 150 base pairs to about 500 base pairs, about 150 base pairs to about 750 base pairs, about 150 base pairs to about 1,000 base pairs, about 200 base pairs to about 250 base pairs, about 200 base pairs to about 300 base pairs, about 200 base pairs to about 350 base pairs, about 200 base pairs to about 400 base pairs, about 200 base pairs to about 450 base pairs, about 200 base pairs to about 500 base pairs, about 200 base pairs to about 750 base pairs, about 200 base pairs to about 1,000 base pairs, about 250 base pairs to about 300 base pairs, about 250 base pairs to about 350 base pairs, about 250 base pairs to about 400 base pairs, about 250 base pairs to about 450 base pairs, about 250 base pairs to about 500 base pairs, about 250 base pairs to about 750 base pairs, about 250 base pairs to about 1,000 base pairs, about 300 base pairs to about 350 base pairs, about 300 base pairs to about 400 base pairs, about 300 base pairs to about 450 base pairs, about 300 base pairs to about 500 base pairs, about 300 base pairs to about 750 base pairs, about 300 base pairs to about 1,000 base pairs, about 350 base pairs to about 400 base pairs, about 350 base pairs to about 450 base pairs, about 350 base pairs to about 500 base pairs, about 350 base pairs to about 750 base pairs, about 350 base pairs to about 1,000 base pairs, about 400 base pairs to about 450 base pairs, about 400 base pairs to about 500 base pairs, about 400 base pairs to about 750 base pairs, about 400 base pairs to about 1,000 base pairs, about 450 base pairs to about 500 base pairs, about 450 base pairs to about 750 base pairs, about 450 base pairs to about 1,000 base pairs, about 500 base pairs to about 750 base pairs, about 500 base pairs to about 1,000 base pairs, or about 750 base pairs to about 1,000 base pairs.

### Site-Specific Nuclease Systems

In the methods provided herein, a nuclease is introduced to the host cell that is capable of causing a double-strand break near or within a genomic target site, which greatly increases the frequency of homologous recombination and HDR at or near the cleavage site. In preferred embodiments, the recognition sequence for the nuclease is present in the host cell genome only at the target site, thereby minimizing any off-target genomic binding and cleavage by the nuclease.

In some embodiments of the methods provided herein, the nuclease is a TAL-effector DNA binding domain-nuclease fusion protein (TALEN). A TAL effector comprises a DNA binding domain that interacts with DNA in a sequence-specific manner through one or more tandem repeat domains. The repeated sequence typically comprises 34 amino acids, and the repeats are typically 91-100% homologous with each other. Polymorphism of the repeats is usually located at positions 12 and 13, and there appears to be a one-to-one correspondence between the identity of repeat variable-diresidues at positions 12 and 13 with the identity of the contiguous nucleotides in the TAL-effector's target sequence. The TAL-effector DNA binding domain may be engineered to bind to a desired target sequence, and fused to a nuclease domain, e.g., from a type II restriction endonuclease, typically a nonspecific cleavage domain from a type II restriction endonuclease such as FokI (see e.g., Kim et al. (1996) Proc. Natl. Acad. Sci. USA 93:1156-1160). Other useful endonucleases may include, for example, HhaI, HindIII, Nod, BbvCI, EcoRI, BglI, and AlwI. Thus, in some embodiments, the TALEN comprises a TAL effector domain comprising a plurality of TAL effector repeat sequences that, in combination, bind to a specific nucleotide sequence in the target DNA sequence, such that the TALEN cleaves the target DNA within or adjacent to the specific nucleotide sequence. TALENS useful for the methods provided herein include those described in WO10/079430 and U.S. Patent Application Publication No. 2011/0145940.

In some embodiments of the methods provided herein, the nuclease is a site-specific recombinase. A site-specific recombinase, also referred to as a recombinase, is a polypeptide that catalyzes conservative site-specific recombination between its compatible recombination sites, and includes native polypeptides as well as derivatives, variants and/or fragments that retain activity, and native polynucleotides, derivatives, variants, and/or fragments that encode a recombinase that retains activity. For reviews of site-specific recombinases and their recognition sites, see, Sauer (1994) Curr Op Biotechnol 5:521-7; and Sadowski, (1993) FASEB 7:760-7. In some embodiments, the recombinase is a serine recombinase or a tyrosine recombinase. In some embodiments, the recombinase is from the Integrase or Resolvase families. In some embodiments, the recombinase is an integrase selected from the group consisting of FLP, Cre, lambda integrase, and R. For other members of the Integrase family, see for example, Esposito, et al., (1997) Nucleic Acids Res 25:3605-14 and Abremski, et al., (1992) Protein Eng 5:87-91.

In some embodiments of the methods provided herein, one or more of the nucleases is a transposase. Transposases are polypeptides that mediate transposition of a transposon from one location in the genome to another. Transposases typically induce double strand breaks to excise the transposon, recognize subterminal repeats, and bring together the ends of the excised transposon, in some systems other proteins are also required to bring together the ends during transposition.

In some embodiments of the methods provided herein, one or more of the nucleases is a zinc-finger nuclease (ZFN). ZFNs are engineered break inducing agents comprised of a zinc finger DNA binding domain and a break inducing agent domain. Engineered ZFNs consist of two zinc finger arrays (ZFAs), each of which is fused to a single subunit of a nonspecific endonuclease, such as the nuclease domain from the FokI enzyme, which becomes active upon dimerization. Typically, a single ZFA consists of 3 or 4 zinc finger domains, each of which is designed to recognize a specific nucleotide triplet (GGC, GAT, etc.). Thus, ZFNs composed of two “3-finger” ZFAs are capable of recognizing an 18 base pair target site; an 18 base pair recognition sequence is generally unique, even within large genomes such as those of humans and plants. By directing the co-localization and dimerization of two FokI nuclease monomers, ZFNs generate a functional site-specific endonuclease that creates a break in DNA at the targeted locus.

### CRISPR-Cas

In some embodiments, the site-specific nuclease system utilizes a nucleic acid-guided nuclease. For example, clustered regularly interspaced short palindromic repeats (CRISPR) and CRISPR-associated (Cas) proteins can be utilized to introduce a targeted double-stranded break in a DNA sequence. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide polynucleotide sequence (also referred to as a “spacer” in the context of an endogenous CRISPR system), and/or other sequences and transcripts from a CRISPR locus.

In some embodiments, the CRISPR/Cas nuclease or CRISPR/Cas nuclease system includes a non-coding RNA molecule (guide) RNA, which sequence-specifically binds to DNA, and a Cas protein (e.g., Cas9), with nuclease functionality (e.g., two nuclease domains).

In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes or Staphylococcus aureus.

In some embodiments, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. In some embodiments, the target site is selected based on its location immediately 5′ of a protospacer adjacent motif (PAM) sequence, such as typically NGG, or NAG. In this respect, the gRNA is targeted to the desired sequence by modifying the first 20 nucleotides of the guide RNA to correspond to the target DNA sequence.

In some embodiments, the CRISPR system induces DSBs at the target site, followed by disruptions as discussed herein. In other embodiments, Cas9 variants, deemed “nickases” are used to nick a single strand at the target site. In some embodiments, paired nickases are used, e.g., to improve specificity, each directed by a pair of different gRNAs targeting sequences such that upon introduction of the nicks simultaneously, a 5′ overhang is introduced.

In general, a CRISPR system is characterized by elements that promote the formation of a CRISPR complex at the site of a target sequence. Typically, in the context of formation of a CRISPR complex, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The target sequence may comprise any polynucleotide, such as DNA polynucleotides. In some embodiments, the target sequence is located in the nucleus or cytoplasm of the cell. In some embodiments, the target sequence may be within an organelle of the cell. Generally, a sequence or template that may be used for recombination into the targeted locus comprising the target sequences is referred to as an “donor template” or “donor polynucleotide” or “donor sequence”. In some embodiments, an exogenous polynucleotide may be referred to as an donor template or donor polynucleotide. In some embodiments, the donor polynucleotide comprises an exogenous polynucleotide sequence. In some embodiments, the recombination is homologous recombination or homology-directed repair (HDR).

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of the CRISPR complex.

As with the target sequence, in some embodiments, complete complementarity is not necessarily needed. In some embodiments, the tracr sequence has at least 50%, 60%, 70%, 80%, 90%, 95% or 99% of sequence complementarity along the length of the tracr mate sequence when optimally aligned. In some embodiments, one or more vectors driving expression of one or more elements of the CRISPR system are introduced into the cell such that expression of the elements of the CRISPR system direct formation of the CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. In some embodiments, CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, the nucleic acid guide programmable nuclease can be a CRISPR enzyme, such as a Cas protein. Non-limiting examples of Cas proteins include Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes, S. aureus or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In some embodiments, a vector encodes a CRISPR enzyme that is mutated to with respect to a corresponding wild-type enzyme. Non-limiting examples of mutations in a Cas9 protein are known in the art (see e.g. WO2015/161276), any of which can be included in a CRISPR/Cas9 system in accord with the provided methods. In some embodiments, the CRISPR enzyme is mutated such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. In some embodiments, one or more codons (e.g. 1, 2, 3, 4, 5, 10, 15, 20, 25, 50, or more, or all codons) in a sequence encoding the CRISPR enzyme corresponds to the most frequently used codon for a particular amino acid.

In general, a guide sequence includes a targeting domain comprising a polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some examples, the targeting domain of the gRNA is complementary, e.g., at least 80, 85, 90, 95, 98 or 99% complementary, e.g., fully complementary, to the target sequence on the target nucleic acid.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of the CRISPR complex to a target sequence may be assessed by any suitable assay. For example, the components of the CRISPR system sufficient to form the CRISPR complex, including the guide sequence to be tested, may be provided to the cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of the CRISPR complex, including the guide sequence to be tested and a control guide sequence different from the test guide sequence, and comparing binding or rate of cleavage at the target sequence between the test and control guide sequence reactions.

A guide sequence may be selected to target any target sequence. In some embodiments, the target sequence is a sequence within a genome of a cell. Exemplary target sequences include those that are unique in the target genome. In some embodiments, a guide sequence is selected to reduce the degree of secondary structure within the guide sequence. Secondary structure may be determined by any suitable polynucleotide folding algorithm.

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences.

Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. In some aspects, loop forming sequences for use in hairpin structures are four nucleotides in length, and have the sequence GAAA. However, longer or shorter loop sequences may be used, as may alternative sequences. In some embodiments, the sequences include a nucleotide triplet (for example, AAA), and an additional nucleotide (for example C or G). Examples of loop forming sequences include CAAA and AAAG. In some embodiments, the transcript or transcribed polynucleotide sequence has at least two or more hairpins. In some embodiments, the transcript has two, three, four or five hairpins. In a further embodiment, the transcript has at most five hairpins. In some embodiments, the single transcript further includes a transcription termination sequence, such as a polyT sequence, for example six T nucleotides.

In some embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CR ISPR enzyme are described in US20110059502, incorporated herein by reference. In some embodiments, a tagged CRISPR enzyme is used to identify the location of a target sequence.

In some embodiments, a CRISPR enzyme in combination with (and optionally complexed with) a guide sequence is delivered to the cell. In some embodiments, methods for introducing a protein component into a cell according to the present disclosure (e.g. Cas9/gRNA RNPs) may be via physical delivery methods (e.g. electroporation, particle gun, Calcium Phosphate transfection, cell compression or squeezing), liposomes or nanoparticles. In some embodiments, target polynucleotides are modified in a eukaryotic cell. In some embodiments, the method comprises allowing the CRISPR complex to bind to the target polynucleotide to effect cleavage of said target polynucleotide thereby modifying the target polynucleotide, wherein the CRISPR complex comprises the CRISPR enzyme complexed with a guide sequence hybridized to a target sequence within said target polynucleotide, wherein said guide sequence is linked to a tracr mate sequence which in turn hybridizes to a tracr sequence.

Binding of the polynucleotide sequence recruits the Cas protein and facilitates a double-stranded break into the polynucleotide sequence by the Cas nuclease. In some embodiments, guide polynucleotide sequence binds to a region of a gene corresponding to the coding sequence. In some embodiments, the coding sequence is an exon. In some embodiments, the guide polynucleotide can bind to a region of the gene corresponding to a non-coding region. In some embodiments, the non-coding region is an intron or untranslated region (UTR).

Guide polynucleotide sequences are specific to the target that they bind. In some embodiments, the guide polynucleotide sequence target is hemoglobin B (HBB). In some embodiments, the guide polynucleotide sequence binds to an exon of HBB. In some embodiments, the guide polynucleotides binds to exon 1, exon 2, or exon 3 of HBB. In a particular embodiment, the guide polynucleotides binds to exon 1 of HBB. In some such embodiments, the guide polynucleotide sequence that binds to HBB exon 1 is SEQ ID NO: 92.

In some embodiments, guide polynucleotide sequence comprises a chemical modification. In some embodiments, the guide polynucleotide sequence comprises a 2′-O-methyl-3′-phosphorothioate modification. Examples of chemical modifications to guide polynucleotide sequences which enhance stability and cleavage efficiency of CRISPR-Cas systems include but are not limited to those described in PCT Publication Nos. WO/2016164356 and WO 2016/089433, each of which is herein incorporated by reference in its entirety.

### Delivery Vectors

Provided herein are delivery vectors that will enable introduction of the gene editig compositions described herein into a cell. The delivery vector may include a surface modification that targets the vector to a cell of the subject, such as an antibody linked to an external surface of the viral delivery vector, wherein the antibody targets hematopoietic stem cells, or precursors thereof. The composition may include a particle (e.g., lipid nanoparticle or liposome) containing the globin gene and the gene editing reagents, or a plurality of lipid nanoparticles having the globin gene and the gene editing reagents comprised or embedded therein. For example, the plurality of lipid nanoparticles may include at least: a first solid lipid nanoparticle comprising a segment of DNA that includes the globin gene; a second solid lipid nanoparticle that includes at least one Cas endonuclease complexed with a guide RNA (gRNA) that targets the Cas endonuclease to a locus within an alpha-globin gene cluster in chromosome 16. The particle(s) may be provided as one or a plurality of liposomes enveloping one or more of the globin gene and the gene editing reagents.

Donor polynucleotide sequences described herein may be incorporated within a wide variety of gene therapy constructs, e.g., to deliver a nucleic acid encoding a protein to a subject in need thereof. A vector construct refers to a polynucleotide molecule including all or a portion of a viral genome and an exogenous polynucleotide sequence. In some instances, gene transfer can be mediated by a DNA viral vector, such as an adenovirus (Ad) or adeno-associated virus (AAV). Other vectors useful in methods of gene therapy are known in the art. For example, a construct of the present invention can include an alphavirus, herpesvirus, retrovirus, lentivirus, or vaccinia virus.

Adenoviruses are a relatively well characterized group of viruses, including over 50 serotypes. Adenoviruses are tractable through the application of techniques of molecular biology and may not require integration into the host cell genome. Recombinant Ad-derived vectors, including vectors that reduce the potential for recombination and generation of wild-type virus, have been constructed. Wild-type AAV has high infectivity and is capable of integrating into a host genome with a high degree of specificity.

AAV of any serotype or pseudotype can be used. Certain AAV vectors are derived from single stranded (ss) DNA parvoviruses that are nonpathogenic for mammals. Briefly, rep and cap viral genes that can account for 96% of the archetypical wild-type AAV genome can be removed in the generation of certain AAV vectors, leaving flanking inverted terminal repeats (ITRs) that can be used to initiate viral DNA replication, packaging and integration. Wild type AAV integrates into the human host cell genome with preferential site specificity at chromosome 19q13.3. Alternatively, AAV can be maintained episomally.

At least twelve human serotypes of AAV (AAV serotype 1 (AAV-1) to AAV-12) and more than 100 serotypes from nonhuman primates have been discovered to date. Any of these serotypes, as well as any combinations thereof, may be used within the scope of the present disclosure.

A serotype of a viral vector used in certain embodiments of the invention can be selected from the group consisting from AAV1, AAV2, AAV3, AAV4, AAV5, AAV6, AAV7, AAV8, and AAV9. Other serotypes are known in the art or described herein and are also applicable to the present disclosure. In particular instances, the present invention includes an AAV9 viral vector including a glucocerebrosidase nucleic acid of the present invention.

A vector of the present invention can be a pseudotyped vector. Pseudotyping provides a mechanism for modulating a vector's target cell population. For instance, pseudotyped AAV vectors can be utilized in various methods described herein. Pseudotyped vectors are those that contain the genome of one vector, e.g., the genome of one AAV serotype, in the capsid of a second vector, e.g., a second AAV serotype. Methods of pseudotyping are well known in the art. For instance, a vector may be pseudotyped with envelope glycoproteins derived from Rhabdovirus vesicular stomatitis virus (VSV) serotypes (Indiana and Chandipura strains), rabies virus (e.g., various Evelyn-Rokitnicki-Abelseth ERA strains and challenge virus standard (CVS)), Lyssavirus Mokola virus, a rabies-related virus, vesicular stomatitis virus (VSV), Mokola virus (MV), lymphocytic choriomeningitis virus (LCMV), rabies virus glycoprotein (RV-G), glycoprotein B type (FuG-B), a variant of FuG-B (FuG-B2) or Moloney murine leukemia virus (MuLV).

Without limitation, illustrative examples of pseudotyped vectors include recombinant AAV2/1, AAV2/2, AAV2/5, AAV2/6, AAV2/7, and AAV2/8 serotype vectors. It is known in the art that such vectors may be engineered to include a transgene encoding a human protein or other protein. In particular instances, the present invention includes a AAV6 vector for delivery.

In some instances, a particular AAV serotype vector may be selected based upon the intended use, e.g., based upon the intended route of administration. For example, for direct injection into the brain, e.g., either into the striatum, an AAV2 serotype vector can be used.

Various methods for application of AAV vector constructs in gene therapy are known in the art, including methods of modification, purification, and preparation for administration to human.

### Genetically Modified Cell

Provided herein is a genetically modified cell, wherein the genetically modified cell is prepared according to the method disclosed herein. The genetically modified cells are prepared by introducing into a cell the programmable nucleic acid-guided nuclease and guide polynucleotide sequence of the disease. In addition, the donor polynucleotide sequence can be administered. Through a single recombination event, at least a portion of the donor polynucleotide sequence is integrated into a region of the target site of the cell.

After targeted gene integration through resolution of a single recombination event between the donor polynucleotide and the endogenous target site, expression of the target gene can be different compared to a cell that has not been genetically modified using the method disclosed in the present disclosure.

In some embodiments, the genetically modified cell has greater expression of a gene following targeted gene insertion compared to a cell that has not been genetically modified. In some embodiments, the genetically modified cell comprises about 50% greater expression to about 100% greater expression compared to a cell that has not been genetically modified. In some embodiments, the genetically modified cell comprises at least about 50% greater expression. In some embodiments, the genetically modified cell comprises at most about 100% greater expression. In some embodiments, the genetically modified cell comprises about 50% greater expression to about 60% greater expression, about 50% greater expression to about 70% greater expression, about 50% greater expression to about 80% greater expression, about 50% greater expression to about 90% greater expression, about 50% greater expression to about 100% greater expression, about 60% greater expression to about 70% greater expression, about 60% greater expression to about 80% greater expression, about 60% greater expression to about 90% greater expression, about 60% greater expression to about 100% greater expression, about 70% greater expression to about 80% greater expression, about 70% greater expression to about 90% greater expression, about 70% greater expression to about 100% greater expression, about 80% greater expression to about 90% greater expression, about 80% greater expression to about 100% greater expression, or about 90% greater expression to about 100% greater expression compared to a cell that has not been genetically modified.

In some embodiments, the genetically modified cell carries the exogenous polynucleotide sequence introduced by the method disclosed herein.

In some embodiments, the genetically modified cell is prepared or generated ex vivo.

In some embodiments, the genetically modified cell is obtained from a subject. In some embodiments, the genetically modified cell is a primary cell. In some embodiments the genetically modified cell is a CD34+ cell. In some embodiments, the genetically modified cell is an HSPC.

### Method of Treatment of Diseases or Disorders

Provided herein are methods of treatment for diseases and disorders.

The term “hemoglobinopathy” or “hemoglobinopathic condition” includes any disorder involving the presence of an abnormal hemoglobin molecule in the blood. Examples of hemoglobinopathies included, but are not limited to, hemoglobin C disease, hemoglobin sickle cell disease (SCD), sickle cell anemia, and thalassemias. Also included are hemoglobinopathies in which a combination of abnormal hemoglobins are present in the blood (e.g., sickle cell/Hb-C disease).

In some embodiments, are compositions administered for the treatment of a disease, wherein the composition treats the aberrant expression of a gene caused by a polymorphism in the endogenously expression polynucleotide sequence. In some embodiments, the disease or disorder is characterized by aberrant expression of a gene. In some embodiments aberrant expression comprises reduced expression or increased expression that results in a manifestation of a disease.

In some embodiments, the disease of disorder is be a hematological disease. In some embodiments, the disease is a hemoglobinopathy. In some embodiments, the disease is β-thalassemia. In some embodiments, the disease is sickle cell disease.

Disease-causing mutations resulting in beta-thalassemia can affect expression of beta-globin (HBB). Mutations can, but are not limited to, perturb transcription, RNA processing, or translation. Mutations affecting transcription can occur in promoter regulatory elements, thereby altering the levels of beta-globin compared to levels of a non-mutated beta-globin gene. Such mutations can affect RNA processing events, such as splicing. Mutations affecting this process can be further stratified into mutations occurring in splice junctions, consensus splice sites, cryptic splice sites the polyA signal, or in the 3′ UTR. Other mutations may affect the translation of the protein, thus affecting the overall characteristics of the protein, such as, but not limited to, the protein's stability. Identified mutations affecting the previously described process have been illustrated in a review of β-thallassemia (Thein, S. L. The Molecular Basis of β-thallasemia. Cold Spring Harbor Perspectives in Medicine. May 13, 2013.).

In other embodiments, the disease is alpha antitrypsin deficiency. α1-antitrypsin deficiency (AATD) is a genetic disorder characterized by a predisposition for the development of a number of diseases, mainly pulmonary emphysema and other chronic respiratory disorders with different clinical manifestations and frequent overlap, and several types of hepatopathies in both children and adults. AAT is the most prevalent proteases inhibitor in the human serum. It is primarily produced in high quantities and secreted mainly by hepatocytes. AAT is an important anti-protease in the lung, but it also has significant anti-inflammatory effects on several cell types and modulates inflammation caused by host and microbial factors. It can play an important role in modulating key immune cell activities and protecting the lungs against damage caused by proteases and inflammation.

Treatment using the compositions and methods of the present disclosure is introduced into a cell. In some embodiments, the cell is obtained from a subject in need of treatment. Cells are contacted with the composition described herein to generate a genetically modified cell with an altered expression profile. The genetically modified cell is re-introduced into the subject to treat the disease or disorder thereof. In some embodiments, the cell is a primary cell. In some embodiments, the cell is a CD34+× cell. In some embodiments, the cell is a hematopoietic stem or progenitor cell. In some embodiments, the cells are obtained from an apheresis product obtained from the donor or subject. In some embodiments, the subject is human.

### Pharmaceutical Compositions

Disclosed herein, in some embodiments, are methods, compositions and kits for use of the modified cells, including pharmaceutical compositions, therapeutic methods, and methods of administration. Although the descriptions of pharmaceutical compositions provided herein are principally directed to pharmaceutical compositions which are suitable for administration to humans, it will be understood by the skilled artisan that such compositions are generally suitable for administration to any animals. In some embodiments, the modified cells of the pharmaceutical composition are autologous to the individual in need thereof. In other embodiments, the modified cells of the pharmaceutical composition are allogeneic to the individual in need thereof.

In some embodiments, a pharmaceutical composition comprising a modified host cell as described herein is provided. In some embodiments, the modified host cell is genetically engineered to comprise an integrated donor sequence, including, for example, diverged coding sequences for a gene of interest, heterologous intron sequences and optionally other regulatory sequences, at a targeted gene locus of the host cell. In some embodiments, a functional diverged donor sequence is integrated into the translational start site of the endogenous gene locus. In some embodiments, the functional diverged donor sequence that is integrated into the host cell genome is expressed under control of the native promoter sequence of the targeted gene locus of the host cell. In some embodiments, the modified host cell is genetically engineered to comprise an integrated functional HBB donor sequence, including, for example, diverged HBB coding sequences and heterologous intron sequences, at the HBB locus. In particular embodiments, a functional diverged HBB donor sequence is integrated into the translational start site of the endogenous HBB locus. In particular embodiments, the functional diverged HBB donor sequence that is integrated into the host cell genome is expressed under control of the native HBB promoter sequence.

In some embodiments, the pharmaceutical composition comprises a plurality of the modified host cells, and further comprises unmodified host cells and/or host cells that have undergone nuclease cleavage resulting in INDELS at the HBB locus but not integration of the diverged HBB donor sequence. In some embodiments, the pharmaceutical composition is comprised of at least 5% of the modified host cells comprising an integrated diverged HBB donor sequence. In some embodiments, the pharmaceutical composition is comprised of about 9% to 50% of the modified host cells comprising an integrated diverged HBB donor sequence. In some embodiments, the pharmaceutical composition is comprised of at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50% or more of the modified host cells comprising an integrated diverged HBB donor sequence. The pharmaceutical compositions described herein may be formulated using one or more excipients to, e.g.: (1) increase stability; (2) alter the biodistribution (e.g., target the cells to specific tissues or cell types, e.g. HSPCs); and/or (3) enhance engraftment in the recipient.

Formulations of the present disclosure can include, without limitation, saline, liposomes, lipid nanoparticles, polymers, peptides, proteins, and combinations thereof. Formulations of the pharmaceutical compositions described herein may be prepared by any method known or hereafter developed in the art of pharmacology. As used herein the term “pharmaceutical composition” refers to compositions including at least one active ingredient (e.g., a modified host cell) and optionally one or more pharmaceutically acceptable excipients. Pharmaceutical compositions of the present disclosure may be sterile.

Relative amounts of the active ingredient (e.g. the modified host cell), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may include between 0.1% and 99% (w/w) of the active ingredient. By way of example, the composition may include between 0.1% and 100%, e.g., between 0.5 and 50%, between 1-30%, between 5-80%, or at least 80% (w/w) active ingredient.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

### Dosing and Administration

The modified host cells of the present disclosure included in the pharmaceutical compositions described above may be administered by any delivery route, systemic delivery or local delivery, which results in a therapeutically effective outcome. These include, but are not limited to, enteral, gastroenteral, epidural, oral, transdermal, intracerebral, intracerebroventricular, epicutaneous, intradermal, subcutaneous, nasal, intravenous, intra-arterial, intramuscular, intracardiac, intraosseous, intrathecal, intraparenchymal, intraperitoneal, intravesical, intravitreal, intracavernous), interstitial, intra-abdominal, intralymphatic, intramedullary, intrapulmonary, intraspinal, intrasynovial, intrathecal, intratubular, parenteral, percutaneous, periarticular, peridural, perineural, periodontal, rectal, soft tissue, and topical. In particular embodiments, the cells are administered intravenously.

In some embodiments, a subject will undergo a conditioning regimen before cell transplantation. For example, before hematopoietic stem cell transplantation, a subject may undergo myeloablative therapy, non-myeloablative therapy or reduced intensity conditioning to prevent rejection of the stem cell transplant even if the stem cell originated from the same subject. The conditioning regime may involve administration of cytotoxic agents. The conditioning regime may also include immunosuppression, antibodies, and irradiation. Other possible conditioning regimens include antibody-mediated conditioning (see, e.g., Czechowicz et al., 318(5854) Science 1296-9 (2007); Palchaudari et al., 34(7) Nature Biotechnology 738-745 (2016); Chhabra et al., 10:8(351) Science Translational Medicine 351ra105 (2016)) and CAR T-mediated conditioning (see, e.g., Arai et al., 26(5) Molecular Therapy 1181-1197 (2018); each of which is hereby incorporated by reference in its entirety). For example, conditioning needs to be used to create space in the brain for microglia derived from engineered hematopoietic stem cells (HSCs) to migrate in to deliver the protein of interest (as in recent gene therapy trials for ALD and MLD). The conditioning regimen is also designed to create niche “space” to allow the transplanted cells to have a place in the body to engraft and proliferate. In HSC transplantation, for example, the conditioning regimen creates niche space in the bone marrow for the transplanted HSCs to engraft. Without a conditioning regimen, the transplanted HSCs cannot engraft.

Certain aspects of the present disclosure are directed to methods of providing pharmaceutical compositions including the modified host cell of the present disclosure to target tissues of mammalian subjects, by contacting target tissues with pharmaceutical compositions including the modified host cell under conditions such that they are substantially retained in such target tissues. In some embodiments, pharmaceutical compositions including the modified host cell include one or more cell penetration agents, although “naked” formulations (such as without cell penetration agents or other agents) are also contemplated, with or without pharmaceutically acceptable excipients.

The present disclosure additionally provides methods of administering modified host cells in accordance with the disclosure to a subject in need thereof. The pharmaceutical compositions including the modified host cell, and compositions of the present disclosure may be administered to a subject using any amount and any route of administration effective for preventing, treating, or managing a hemoglobinopathy or other disease described herein. The exact amount required will vary from subject to subject, depending on the species, age, and general condition of the subject, the severity of the disease, the particular composition, its mode of administration, its mode of activity, and the like. The subject may be a human, a mammal, or an animal. The specific therapeutically or prophylactically effective dose level for any particular individual will depend upon a variety of factors including the disorder being treated and the severity of the disorder; the activity of the specific payload employed; the specific composition employed; the age, body weight, general health, sex and diet of the patient; the time of administration, route of administration; the duration of the treatment; drugs used in combination or coincidental with the specific modified host cell employed; and like factors well known in the medical arts.

In certain embodiments, modified host cell pharmaceutical compositions in accordance with the present disclosure may be administered at dosage levels sufficient to deliver from, e.g., about 1×10⁴ to 1×10⁵, 1×10⁵ to 1×10⁶, 1×10⁶ to 1×10⁷, or more cells to the subject, or any amount sufficient to obtain the desired therapeutic or prophylactic, effect. The desired dosage of the modified host cell pharmaceutical compositions of the present disclosure may be administered one time or multiple times. In some embodiments, delivery of the modified host cell to a subject provides a therapeutic effect for at least 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 20 months, 21 months, 22 months, 23 months, 2 years, 3 years, 4 years, 5 years, 6 years, 7 years, 8 years, 9 years, 10 years or more than 10 years. In some embodiments, only a single dose is needed to effect treatment or prevention of a disease or disorder described herein. In other embodiments, a subject in need thereof may receive more than one dose, for example, 2, 3, or more than 3 doses of a modified host cell pharmaceutical compositions described herein to effect treatment or prevention of the disease or disorder.

The modified host cells may be used in combination with one or more other therapeutic, prophylactic, research or diagnostic agents, or medical procedures, either sequentially or concurrently. In general, each agent will be administered at a dose and/or on a time schedule determined for that agent.

Use of a modified mammalian host cell according to the present disclosure for treatment of a hemoglobinopathy or other disease described herein is also encompassed by the disclosure.

The present disclosure also contemplates kits comprising compositions or components of the present disclosure, e.g., sgRNA, Cas nuclease, RNPs, and/or homologous templates, as well as, optionally, reagents for, e.g., the introduction of the components into cells. The kits can also comprise one or more containers or vials, as well as instructions for using the compositions in order to modify cells and treat subjects according to the methods described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1: Evaluation of Beta-Hemoglobin Expression Levels from Two Different Loci

In the examples described below, the levels of beta hemoglobin expression from two different loci were assessed using following methods.

### AAV Production:

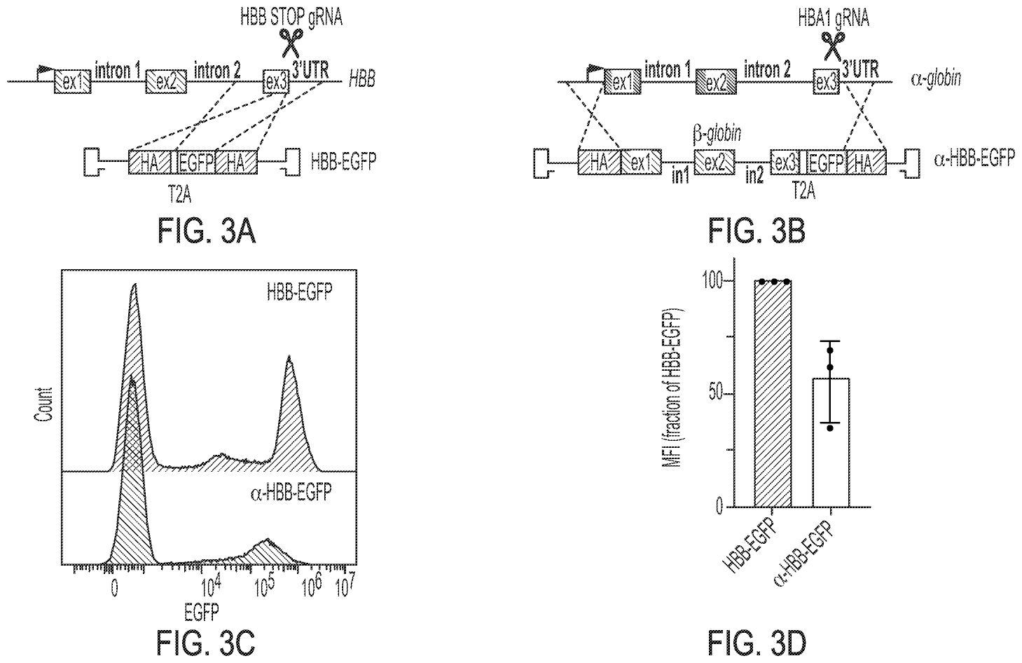

All AAV6 vectors were cloned into the pAAV-MCS plasmid (Agilent Technologies, Santa Clara, Calif., USA), which contain inverted terminal repeats (ITRs) derived from AAV2. Left and right homology arms (LHAs/RHAs) were PCR amplified from human genomic DNA to match the indicated length at the respective knock-in sites (see FIGS. 3-5). 293FT cells (Thermo Fisher) were seeded in Millicell HY multilayer flasks (EMD) with ˜12.5×10⁷ cells per flask. 24 hours later, each dish was transfected with a standard polyethylenimine (PEI) transfection of 60 μg ITR-containing plasmid and 220 μg pDP6 (Plasmid Factory GmbH), which contains the AAV6 cap genes, AAV2 rep genes, and Ad5 helper genes. After a 48-72 hour incubation, cells were harvested and AAV purified using an AAVPro Purification Kit (All Serotypes) (Takara Bio USA) to extract full AAV6 capsids as per manufacturer's instructions. AAV6 vectors were then titered using ddPCR to measure number of vector genomes and calculate vector genomes per cell.

CD34+ HSPCs culture:

CD34+ HSPCs were purchased from AllCells and were isolated from G-CSF-mobilized peripheral blood from healthy donors. SCD-CD34+ HSPCs were obtained from patients with sickle cell disease. CD34+ HSPCs were cultured at 2.5×10⁵-5×10⁵ cells/mL in GMP SCGM Stem Cell Growth Medium (CellGenix) supplemented with stem cell factor (SCF)(100 ng/mL), thrombopoietin (TPO)(100 ng/mL) (Peprotech), FLT3-ligand (100 ng/mL) (Peprotech), IL-6 (100 ng/mL) (Peprotech) and UM171 (35 nM) (Selleckchem). Cells were cultured at 37° C., 5% CO₂, and 5% O₂.

### Genome Editing of CD34+ HSPCs:

Chemically-modified sgRNAs used to edit CD34+ HSPCs at either HBA1 or HBB were purchased from Synthego. The sgRNA modifications added were 2′-O-methyl-3′-phosphorothioate at the three terminal nucleotides of the 5′ and 3′ ends. The target sequences for sgRNAs were as follows: HBA1: 5′-GGCAAGAAGCATGGCCACCG-3′ (SEQ ID NO: 25); HBB-STOP: 5′-AGCGAGCTTAGTGATACTTG-3′ (SEQ ID NO: 26); HBB-EXON 1: 5′-CTTGCCCCACAGGGCAGTAA-3′ (SEQ ID NO: 27). Cas9 protein (SpyFi Cas9) was purchased from Aldevron. The RNPs were complexed at a Cas9: sgRNA molar ratio of 1:2.5 at 25° C. for 10-15 minutes prior to electroporation. CD34+ cells were resuspended in P3 buffer (Lonza, Basel, Switzerland) with complexed RNPs and electroporated using a Lonza 4D Nucleofector (program DZ-100) and 20 μl cuvettes. After electroporation, cells were plated at 2.5×10⁵ cells/mL in the cytokine-supplemented media described above that contained the respective AAV6 particles. AAV6 was supplied to the cells at 2.5×10³-5×10³ vector genomes/cell based on titers determined by ddPCR.

In Vitro Differentiation of CD34+ HSPCs into Erythrocytes:

One day post electroporation, AAV containing media was removed and HSPCs were cultured for 7 days at 37° C. and 5% CO2 in SFEM II medium (STEMCELL Technologies) supplemented with Erythroid Expansion Supplement (STEMCELL Technologies) at a density of 5-10×10⁴ cells/mL. At day 7, cells were transferred to a secondary differentiation medium in which SFEM II was supplemented with 10 ng/mL SCF (Peprotech), 3 U/mL erythropoietin (Peprotech), 200 μg/mL transferrin (Sigma-Aldrich) and 3% human AB serum (Sigma Aldrich) and cells were cultured for an additional 3 days at a density of 1×10⁵ cells/mL before subjecting them to flow cytometry for EGFP expression at day 10.

### Flow Cytometry of Differentiated Erythroblasts:

HSPCs subjected to erythrocyte differentiation after genome editing were analyzed at day 10 for erythrocyte lineage-specific markers using a Cytoflex cytometer (Beckman Coulter). Edited and non-edited cells were analyzed by flow cytometry using the following antibodies: hCD45 V450 (HI30; BD Biosciences), CD34 APC (561; BioLegend), CD71 PE-Cy7 (OKT9; Affymetrix), and CD235a PE (GPA)(GA-R2; BD Biosciences). Cells were harvested and resuspended in PBS with 0.5% BSA containing the listed antibodies and a live/dead cell stain (Ghost dye 780, Cell Signaling). Cells were incubated with staining solution for 30 minutes at room temperature and then washed with PBS. Cells were resuspended in PBS with 0.5% BSA and subjected to flow cytometry. Analysis was performed using FlowJo Software. During analysis cells were gated for single cells, live cells, CD34−/CD45− cells and then for GPA+/CD71+ cells to distinguished successfully differentiated erythroblasts from more stem-like progenitors. Targeting rates were determined by gating for GFP positive cells within the population of GPA+/CD71+ cells. The mean fluorescence intensity was determined from the GFP+ gate and serves as a measure for protein expression levels from edited alleles.

### Results:

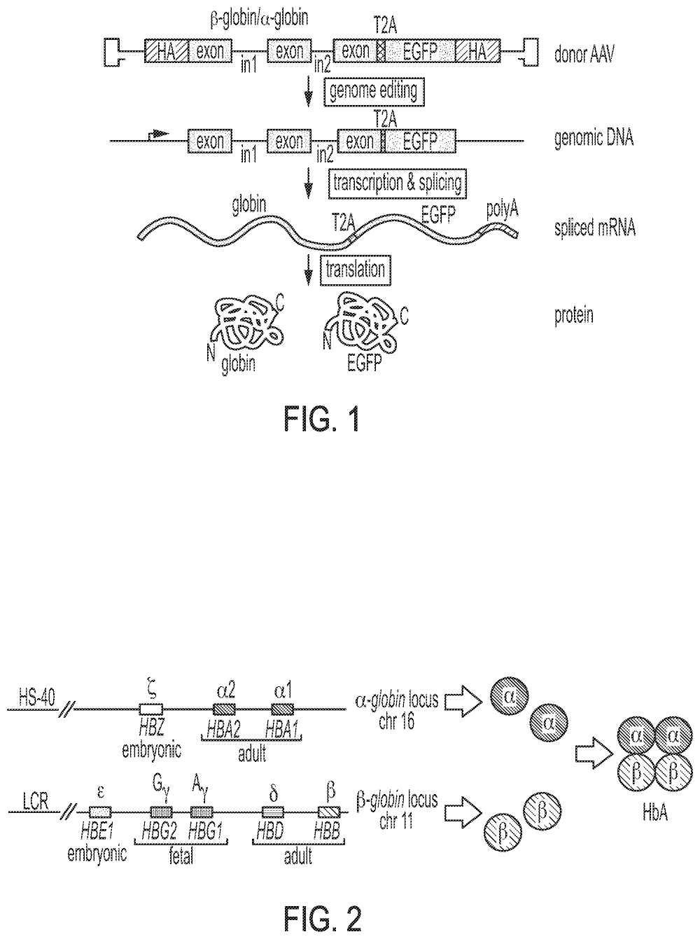

To assess the relative expression of HBB in its endogenous or heterologous locus, an EGFP reporter system was used to serve as a proxy for hemoglobin expression in either of these loci. In this system, AAV6 donor templates were designed to contain a T2A-EGFP sequence adjoining the 3′ end of the coding sequence of beta-globin, along with homology arms (HA) to either HBB (5′ HA: intron 2/exon 3 (SEQ ID NO: 21); 3′ HA: 3′UTR (SEQ ID NO: 22)); (“Construct 1”) or HBA1 (5′ HA: promoter/5′UTR (SEQ ID NO: 23); 3′ HA: 3′UTR (SEQ ID NO: 24)) (“Construct 2”). Integration of Construct 1 introduces EGFP to the 3′ end of the endogenous HBB gene (FIG. 3A), while integration of Construct 2 results in replacement of the HBA1 gene (exon 1 to exon 3) with HBB-T2A-EGFP (FIG. 3B), including HBB intronic sequences (SEQ ID NOs: 9-10). After successful knock-in to the HBB and HBA1 loci, expression of HBB-T2A-EGFP is driven by the endogenous HBB and HBA1 promoter, respectively. HBB and EGFP are transcribed as a single mRNA, and during translation the proteins are cleaved in the ribosomes at the T2A site. As the translation of every transcript results in stochiometric amounts of β-globin to EGFP, thus the amount of EGFP protein produced is directly correlative to β-globin expression levels (FIG. 1).

The α-globin genes are duplicated genes located on chromosome 16 (HBA1 and HBA2), while the β-globin gene is a single gene on chromosome 11, but the stochiometric ratio of α- to β-globin is approximately 1:1 in adult erythroid cells (FIG. 2). To assess the relative expression levels of HBB in the HBB and the HBA1 locus, respectively, HSPCs were modified at either: (1) the 3′ end of the HBB gene, using CRISPR-Cas9 RNP (with sgRNA targeting HBB-STOP (SEQ ID NO: 26) and AAV6 donor Construct 1 to endogenously tag HBB with EGFP (HBB-EGFP), FIG. 3A); or (2) at the HBA1 locus, using CRISPR-Cas9 RNP (with sgRNA targeting HBA1 (SEQ ID NO: 25) and AAV6 donor Construct 2 to replace the HBA1 gene with an exogenous copy of HBB tagged with EGFP (α-HBB-EGFP, FIG. 3B). After genome editing, HSPCs underwent erythroid differentiation for ten days, and were then analyzed for EGFP fluorescence by flow cytometry to compare the expression of HBB-EGFP to expression of α-HBB-EGFP.

As shown in FIG. 3C and FIG. 3D, HBB-EGFP expressing cells appeared approximately two-fold brighter than α-HBB-EGFP cells as quantified by mean fluorescence intensity (MFI). These data indicate that the endogenous HBB promoter is more powerful than the HBA1 promoter when trying to maximize beta-globin expression, and that gene replacement at the HBB locus would be preferred.

### Example 2: Diverged Coding Sequences of Beta-Hemoglobin

While HBB gene replacement at the HBB locus may be advantageous over addition of a HBB gene copy at the HBA1 locus, homology of the AAV6 donor to the target site may result in undesired recombination events and partial homologous recombination if the wild-type HBB gene sequence is used. Ideally, gene correction or replacement of mutations over longer stretches of DNA, such as those seen in beta-thalassemia major, would use a single gRNA, would avoid homology concerns of the AAV6 donor, and would preserve the strong endogenous regulation of the target gene from its native promoter.

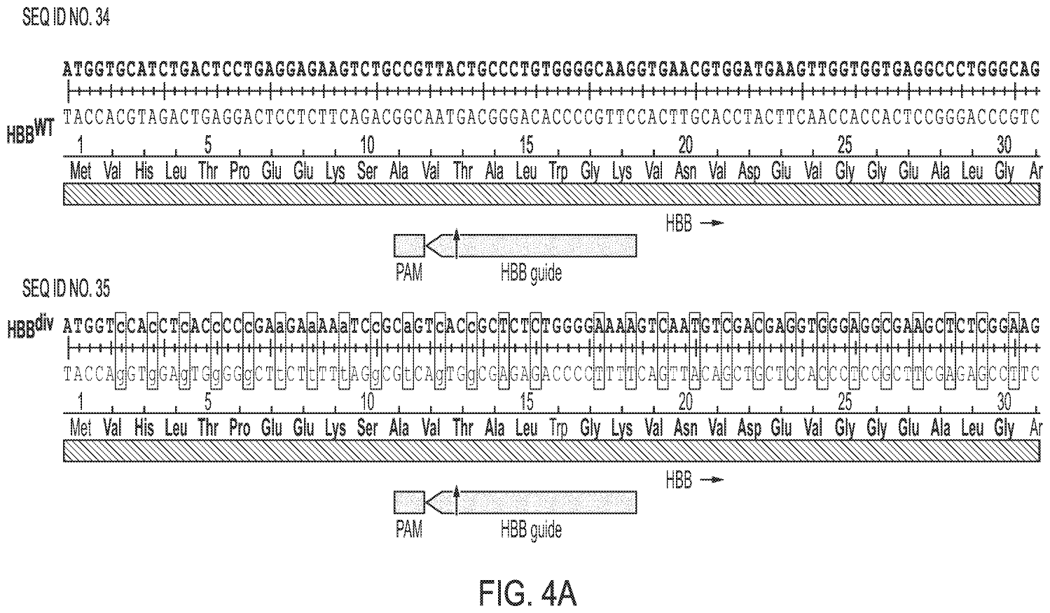

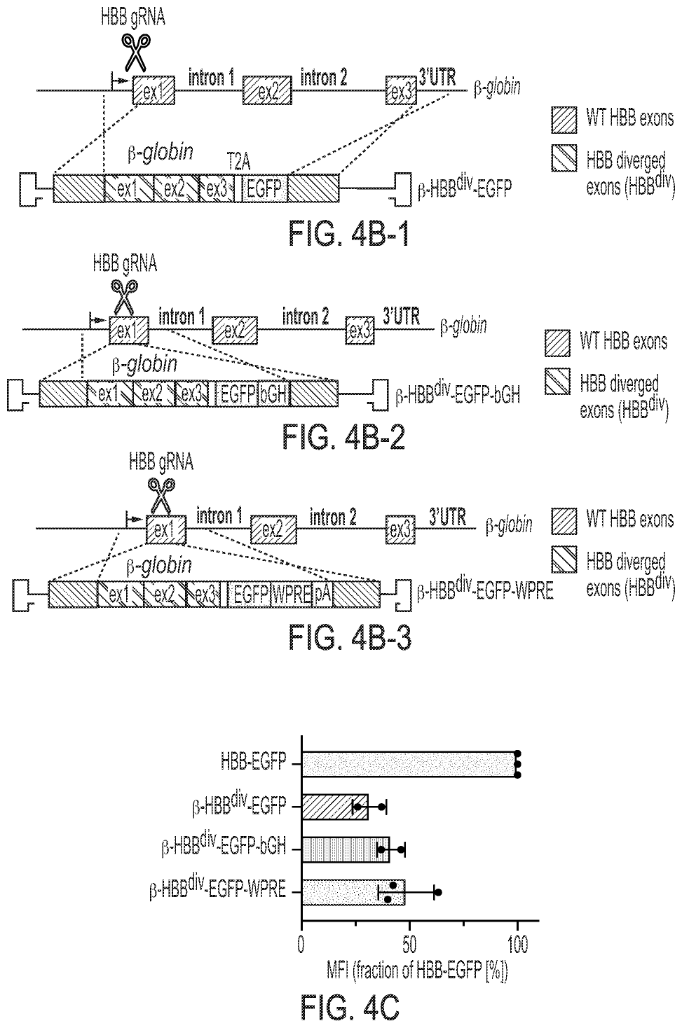

Accordingly, a strategy using CRISPR-Cas9 and AAV6 donors that uses a single gRNA was developed to circumvent the homology concerns of the AAV6 donor. First, starting at the HBB start codon, the beta-globin coding sequence was diverged from the wild-type coding sequence by choosing alternative codons for each amino acid whenever possible without changing the translation of the codon to achieve minimal transgene homology to the target insertion site (FIG. 4A).

A codon usage table was used as a guide to choose the most common or, if the most common codon was the wild-type codon, the second-most common codon for translation in human cells. As shown below, a global sequence alignment using Needle (EMBOSS), based on the Needleman-Wunsch algorithm, identifies the sequence changes made to diverge the HBB sequence (SEQ ID NO: 8), thereby decreasing the sequence identity to 66% with the wild-type (WT) HBB nucleotide sequence (SEQ ID NO: 7), while coding for the same protein sequence.

| | | | |
|---|---|---|---|
| WT | 1 | ATGGTGCATCTGACTCCTGAGGAGAAGTCTGCCGTTACTGCCCTGTGGGG | 50 |
| | | \|\|\|\|\|.\|\|.\|\|.\|\|.\|\|.\|\|.\|\|.\|\|.\|\|.\|\|.\|\|.\|\|.\|\|.\|\|.\|\|\|\|\| | |
| Diverged | 1 | ATGGTCCACCTCACCCCCGAAGAAAAATCCGCAGTCACCGCTCTCTGGGG | 50 |
| WT | 51 | CAAGGTGAACGTGGATGAAGTTGGTGGTGAGGCCCTGGGCAGGCTGCTGG | 100 |
| | | .\|\|.\|\|.\|\|.\|\|.\|\|.\|\|.\|\|.\|\|.\|\|.\|\|.\|\|.\|\|.\|\|.\|\|.\|\|.\|\|.\| | |
| Diverged | 51 | AAAAGTCAATGTCGACGAGGTGGGAGGCGAAGCTCTCGGAAGACTCCTCG | 100 |
| WT | 101 | TGGTCTACCCTTGGACCCAGAGGTTCTTTGAGTCCTTTGGGGATCTGTCC | 150 |
| | | \|.\|\|.\|\|.\|\|.\|\|\|\|\|.\|\|.\|\|.\|\|.\|\|.\|\|...\|\|\|.\|\|.\|\|.\|\|...\| | |
| Diverged | 101 | TCGTGTATCCCTGGACACAAAGATTTTTCGAAAGCTTCGGCGACCTCAGC | 150 |
| WT | 151 | ACTCCTGATGCTGTTATGGGCAACCCTAAGGTGAAGGCTCATGGCAAGAA | 200 |
| | | \|\|.\|\|.\|\|.\|\|.\|\|.\|\|\|\|\|.\|\|.\|\|.\|\|.\|\|.\|\|.\|\|.\|\|.\|\|.\|\|.\|\| | |
| Diverged | 151 | ACACCCGACGCCGTGATGGGGAATCCCAAAGTCAAAGCCCACGGGAAAAA | 200 |
| WT | 201 | AGTGCTCGGTGCCTTTAGTGATGGCCTGGCTCACCTGGACAACCTCAAGG | 250 |
| | | .\|\|.\|\|.\|\|.\|\|.\|\|....\|\|.\|\|.\|\|.\|\|.\|\|.\|\|.\|\|.\|\|.\|\|.\|\|.\| | |
| Diverged | 201 | GGTCCTGGGCGCTTTCTCCGACGGACTCGCCCATCTCGATAATCTGAAAG | 250 |
| WT | 251 | GCACCTTTGCCACACTGAGTGAGCTGCACTGTGACAAGCTGCACGTGGAT | 300 |
| | | \|.\|\|.\|\|.\|\|.\|\|.\|\|....\|\|.\|\|.\|\|.\|\|.\|\|.\|\|.\|\|.\|\|.\|\|.\|\|. | |
| Diverged | 251 | GAACTTTCGCTACCCTCTCCGAACTCCATTGCGATAAACTCCATGTCGAC | 300 |
| WT | 301 | CCTGAGAACTTCAGGCTCCTGGGCAACGTGCTGGTCTGTGTGCTGGCCCA | 350 |
| | | \|\|.\|\|.\|\|.\|\|.\|\|.\|\|.\|\|.\|\|.\|\|.\|\|.\|\|.\|\|.\|\|.\|\|.\|\|.\|\|.\|\| | |
| Diverged | 301 | CCCGAAAATTTTAGACTGCTCGGGAATGTCCTCGTGTGCGTCCTCGCTCA | 350 |
| WT | 351 | TCACTTTGGCAAAGAATTCACCCCACCAGTGCAGGCTGCCTATCAGAAAG | 400 |
| | | .\|\|.\|\|.\|\|.\|\|.\|\|.\|\|.\|\|.\|\|.\|\|.\|\|.\|\|.\|\|.\|\|.\|\|.\|\|.\|\|.\| | |
| Diverged | 351 | CCATTTCGGGAAGGAGTTTACACCCCCCGTCCAAGCCGCTTACCAAAAGG | 400 |
| WT | 401 | TGGTGGCTGGTGTGGCTAATGCCCTGGCCCACAAGTATCAC | 441 |
| | | \|.\|\|.\|\|.\|\|.\|\|.\|\|.\|\|.\|\|.\|\|.\|\|.\|\|.\|\|.\|\|.\|\|. | |
| Diverged | 401 | TCGTCGCCGGAGTCGCCAACGCTCTCGCTCATAAATACCAT | 441 |

The diverged coding sequences were synthesized as gene fragments (Twist Bioscience or Genewiz) and cloned into pAAV with LHA and RHA via Gibson assembly (New England Labs). The methods used in this example are previously described in EXAMPLE 1.

Several different knock-in strategies were tested for inserting the diverged HBB coding sequence (without introns) into the HBB locus. Three donor constructs were designed: (1) β-HBB^{div}-EGFP (FIG. 4B(i)); (2) β-HBB^{div}-EGFP-bGH (FIG. 4B(ii)), which utilizes the bovine growth hormone polyadenylation sequence (SEQ ID NO: 32); and (3) β-HBB^{div}-EGFP-WPRE (FIG. 4B(iii)), which utilizes the woodchuck hepatitis virus post-transcriptional response element (SEQ ID NO: 33). For β-HBB^{div}-EGFP (FIG. 4B(i)), 5′ and 3′ homology arms (SEQ ID NO: 17 and SEQ ID NO: 18, respectively) were designed to facilitate HDR of the HBB locus, resulting in replacement of the endogenous HBB locus downstream of the HBB promoter. An additional strategy was explored for the β-HBB^{div}-EGFP-bGH (FIG. 4B (ii)) and β-HBB^{div}-EGFP-WPRE (FIG. 4B (iii)) donor constructs, in which the 5′ and 3′ homology arms were designed to be homologous to the 5′ UTR (SEQ ID NO: 19) and intron 1 (SEQ ID NO: 20) of HBB, such that HDR would result in insertion of the donor construct downstream from the HBB promoter (and replacement of endogenous exon 1), while leaving the endogenous HBB intron 1, exon 2, intron 2 and exon 3 intact but not expressed.

HSPCs were modified as follows: (1) at the 3′ end of the HBB gene, using CRISPR-Cas9 RNP (with sgRNA targeting HBB-STOP (SEQ ID NO: 26) and AAV6 donor Construct 1 to endogenously tag HBB with EGFP (HBB-EGFP), FIG. 3A); or (2) at the HBB locus, using CRISPR-Cas9 RNP (with sgRNA targeting HBB exon 1 (SEQ ID NO: 27) and either β-HBB^{div}-EGFP (FIG. 4B(i)), β-HBB^{div}-EGFP-bGH (FIG. 4B (ii)), or β-HBB^{div}-EGFP-WPRE (FIG. 4B (iii)). After genome editing, HSPCs underwent erythroid differentiation for ten days, then analyzed for EGFP fluorescence by flow cytometry.

As shown in FIG. 4C, when comparing expression levels of EGFP between the HBB-EGFP control (FIG. 3A) and each of the three β-HBB^{div}-EGFP constructs (FIG. 4B), it was found that expression levels were significantly lower in cells edited with the β-HBB^{div}-EGFP constructs. Thus, additional regulatory elements or introns may be required to increase expression of the HBB^{div} donor sequences closer to physiological levels.

### Example 3: Incorporation of Heterologous Introns Boost HBB-T2A-EGFP Expression to Physiological Levels in CD34-Derived RBCs

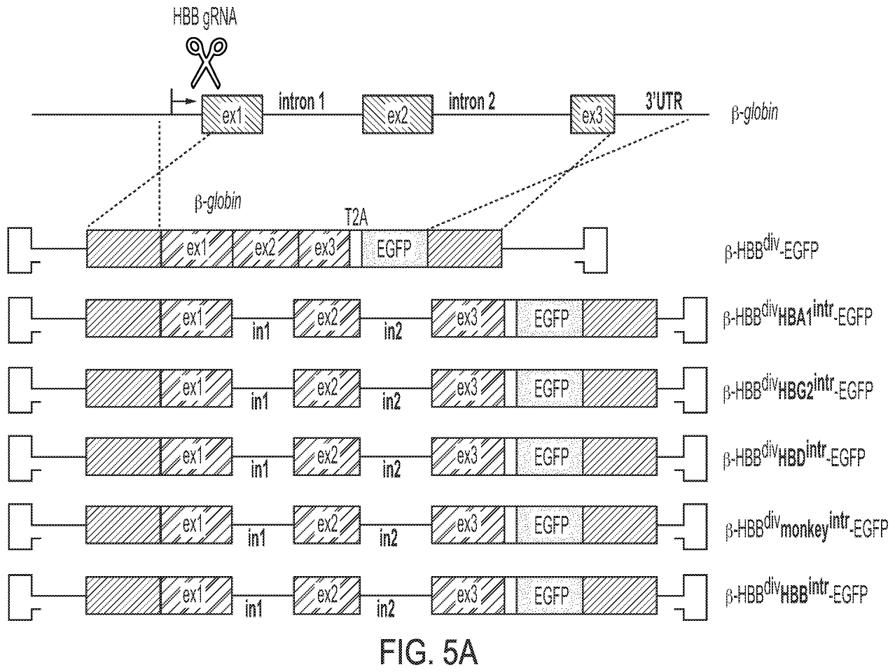

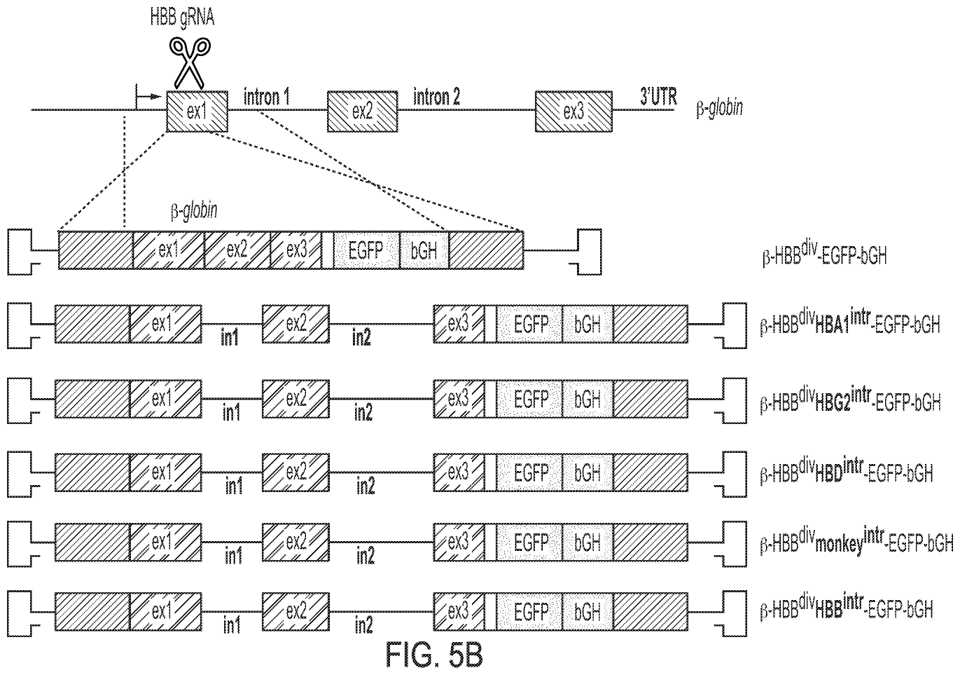

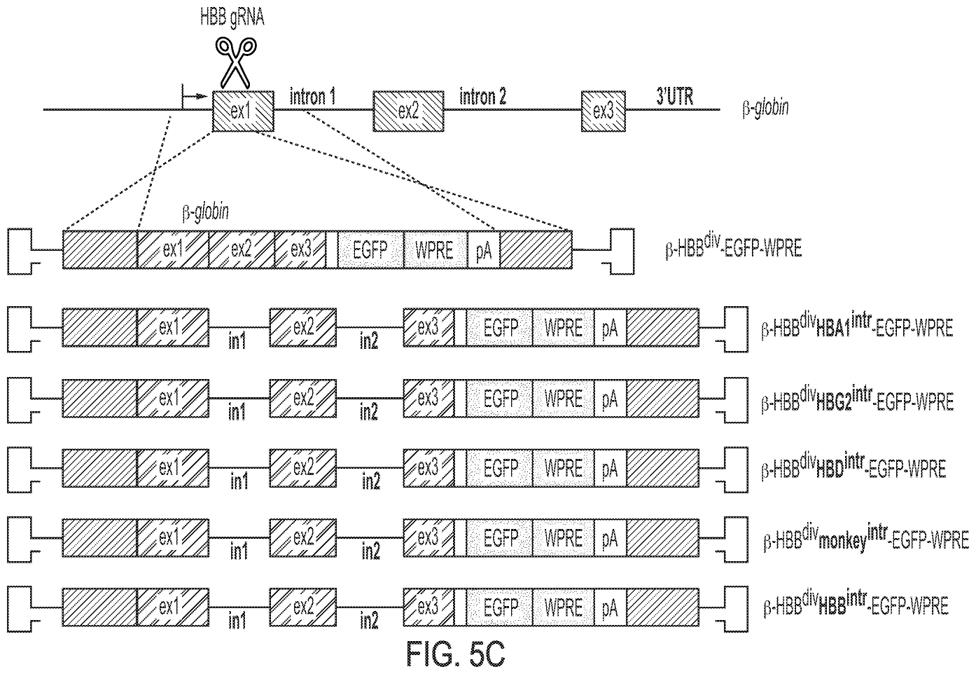

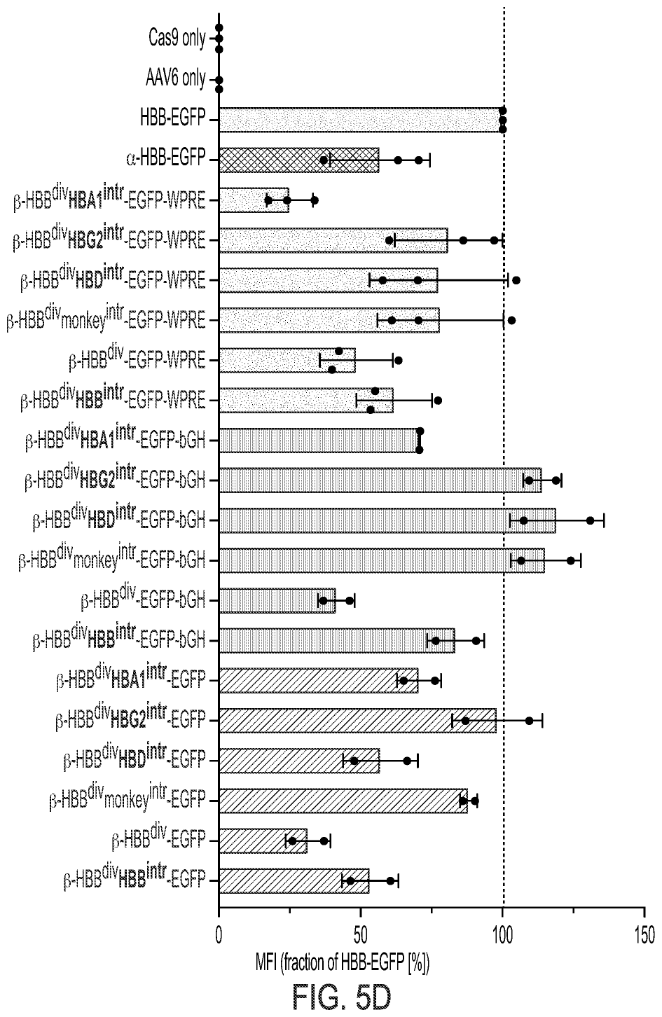

As all hemoglobin genes have a highly similar three exon—two intron structure, we surmised that adding introns from other hemoglobin genes might boost expression levels, as pre-mRNA processing and splicing may be maintained. Thus, AAV6 donors were developed to contain the diverged HBB coding sequence (linked to T2A-EGFP) and to further include HBB intronic sequences, as well as intronic sequences from other hemoglobin genes (HBA1 (SEQ ID NOs: 28-29), HBG2 (SEQ ID NOs: 11-12), and HBD (SEQ ID NOs: 13-14)), and HBD introns from non-human primates, which have sequence similarity but are not completely homologous to human HBB or HBD introns (FIGS. 5A-5C). The first intron from non-human primates was generated by aligning the hemoglobin intron sequences of gibbon, gorilla, chimp, bonobo, orangutan and marmoset to the intron sequences of human HBB. Identified SNPs were then introduced into the human HBB intronic sequence to generate composite “monkey” intron sequences (SEQ ID NOs: 15-16) that were diverged as much as possible from the human HBB intron sequences. Intron 2 from HBD gibbon had very little homology to the human HBB gene and was used as the intron 2 sequence for the composite “monkey” construct. Additional constructs were designed to test the diverged HBB plus heterologous intron sequences in tandem with 3′ bGH polyadenylation and WPRE sequences, respectively. Two knock-in strategies were tested for inserting the diverged HBB coding sequence with heterologous introns into the HBB locus. For constructs without a 3′ regulatory sequence, homology arms were designed to facilitate replacement of the endogenous HBB locus while maintaining native 3′ HBB regulatory sequences and the UTR. For constructs containing exogenous 3′ regulatory sequences, homology arms were designed such that HDR would result in insertion of the donor construct distal to the promoter of HBB (and replacement of endogenous exon 1), while leaving the endogenous HBB exon 2 and exon 3 intact but not expressed.

Table 2 summarizes the AAV6 donor constructs utilized in this study.

TABLE 2
HBB donor constructs containing heterologous introns

| | HBB | | | | |
|---|---|---|---|---|---|
| | diverged | | 3′ regulatory | Homology | |
| Construct name | exons | Introns | sequence | arms | FIG. |
| β-HBB^{div}- EGFP | SEQ ID | none | none | HBB 5′ & 3′ | 5A |
| | NOs: 35- | | | (SEQ ID | |
| | 37 | | | NOs: 17-18) | |
| β-HBB^{div}HBA1^{intr}-EGFP | SEQ ID | HBA1 (SEQ | none | HBB 5′ & 3′ | 5A |
| | NOs: 35- | ID NOs: 28- | | (SEQ ID | |
| | 37 | 29) | | NOs: 17-18) | |
| β-HBB^{div}HBG2^{intr}-EGFP | SEQ ID | HBG2 (SEQ | none | HBB 5′ & 3′ | 5A |
| | NOs: 35- | ID NOs: 11- | | (SEQ ID | |
| | 37 | 12) | | NOs: 17-18) | |
| β-HBB^{div}HBD^{intr}-EGFP | SEQ ID | HBD (SEQ | none | HBB 5′ & 3′ | 5A |
| | NOs: 35- | ID NOs: 13- | | (SEQ ID | |
| | 37 | 14) | | NOs: 17-18) | |
| β-HBB^{div}monkey^{intr}-EGFP | SEQ ID | Monkey | none | HBB 5′ & 3′ | 5A |
| | NOs: 35- | (SEQ ID | | (SEQ ID | |
| | 37 | NOs: 15-16) | | NOs: 17-18) | |
| β-HBB^{div}HBB^{intr}-EGFP | SEQ ID | HBB (SEQ | none | HBB 5′ & 3′ | 5A |
| | NOs: 35- | ID NOs: 9- | | (SEQ ID | |
| | 37 | 10) | | NOs: 17-18) | |
| β-HBB^{div}-EGFP-bGH | SEQ ID | none | bGH poly A | HBB 5′ & | 5B |
| | NOs: 35- | | (SEQ ID NO: | ex1 | |
| | 37 | | 32) | (SEQ ID | |
| | | | | NOs: 19-20) | |
| β-HBB^{div}HBA1^{intr}-EGFP- | SEQ ID | HBA1 (SEQ | bGH poly A | HBB 5′ & | 5B |
| bGH | NOs: 35- | ID NOs: 28- | (SEQ ID NO: | int1 | |
| | 37 | 29) | 32) | (SEQ ID | |
| | | | | NOs: 19-20) | |
| β-HBB^{div}HBG2^{intr}-EGFP- | SEQ ID | HBG2 (SEQ | bGH poly A | HBB 5′ & | 5B |
| bGH | NOs: 35- | ID NOs: 11- | (SEQ ID NO: | int1 | |
| | 37 | 12) | 32) | (SEQ ID | |
| | | | | NOs: 19-20) | |
| β-HBB^{div}HBD^{intr}-EGFP- | SEQ ID | HBD (SEQ | bGH poly A | HBB 5′ & | 5B |
| bGH | NOs: 35- | ID NOs: 13- | (SEQ ID NO: | int1 | |
| | 37 | 14) | 32) | (SEQ ID | |
| | | | | NOs: 19-20) | |
| β-HBB^{div}monkey^{intr}- | SEQ ID | Monkey | bGH poly A | HBB 5′ & | 5B |
| EGFP-bGH | NOs: 35- | (SEQ ID | (SEQ ID NO: | int1 | |
| | 37 | NOs: 15-16) | 32) | (SEQ ID | |
| | | | | NOs: 19-20) | |
| β-HBB^{div}HBB^{intr}-EGFP- | SEQ ID | HBB (SEQ | bGH poly A | HBB 5′ & | 5B |
| bGH | NOs: 35- | ID NOs: 9- | (SEQ ID NO: | int1 | |
| | 37 | 10) | 32) | (SEQ ID | |
| | | | | NOs: 19-20) | |
| β-HBB^{div}-EGFP-WPRE | SEQ ID | none | WPRE | HBB 5′ & | 5C |
| | NOs: 35- | | (SEQ ID NO: | int1 | |
| | 37 | | 33) | (SEQ ID | |
| | | | | NOs: 19-20) | |
| β-HBB^{div}HBA1^{intr}-EGFP- | SEQ ID | HBA1 (SEQ | WPRE | HBB 5′ & | 5C |
| WPRE | NOs: 35- | ID NOs: 28- | (SEQ ID NO: | int1 | |
| | 37 | 29) | 33) | (SEQ ID | |
| | | | | NOs: 19-20) | |
| β-HBB^{div}HBG2^{intr}-EGFP- | SEQ ID | HBG2 (SEQ | WPRE | HBB 5′ & | 5C |
| WPRE | NOs: 35- | ID NOs: 11- | (SEQ ID NO: | int1 | |
| | 37 | 12) | 33) | (SEQ ID | |
| | | | | NOs: 19-20) | |
| β-HBB^{div}HBD^{intr}-EGFP- | SEQ ID | HBD (SEQ | WPRE | HBB 5′ & | 5C |
| WPRE | NOs: 35- | ID NOs: 13- | (SEQ ID NO: | int1 | |
| | 37 | 14) | 33) | (SEQ ID | |
| | | | | NOs: 19-20) | |
| β-HBB^{div}monkey^{intr}- | SEQ ID | Monkey | WPRE | HBB 5′ & | 5C |
| EGFP-WPRE | NOs: 35- | (SEQ ID | (SEQ ID NO: | int1 | |
| | 37 | NOs: 15-16) | 33) | (SEQ ID | |
| | | | | NOs: 19-20) | |
| β-HBB^{div}HBB^{intr}-EGFP- | SEQ ID | HBB (SEQ | WPRE | HBB 5′ & | 5C |
| WPRE | NOs: 35- | ID NOs: 9- | (SEQ ID NO: | int1 | |
| | 37 | 10) | 33) | (SEQ ID | |
| | | | | NOs: 19-20) | |

After genome editing of CD34+ HSPCs utilizing the above constructs, edited HSPCs underwent erythroid differentiation for ten days, then analyzed for EGFP fluorescence by flow cytometry. As shown in FIG. 5D, editing of HSPCs at the HBB locus with donor constructs containing heterologous introns resulted in significantly increased HBB-EGFP expression compared to when intron-less donor constructs were used (except for heterologous HBA1 introns when utilized with a WPRE 3′ regulatory sequence). Inclusion of certain heterologous introns (HBG2, HBD, monkey) induced expression of HBB-EGFP near or above levels observed when the endogenous HBB locus was tagged with EGFP (which is representative of physiological HBB expression, as described in Example 1), particularly when combined with bGH and WPRE 3′ regulatory sequences. These results demonstrate the effectiveness of donor constructs containing diverged coding sequences and heterologous intron sequences for HDR-based gene-correction or replacement applications.

### Example 4: Heterologous Introns Boost HBB Expression in CD34-Derived Red Blood Cells

For the following examples describing HDR rates and expression levels of optimized HBB^{div} donor, the following methods were used, in addition to methods similar to those described in Example 1.

### Immunophenotyping of CD34-Derived RBCs

HSPCs subjected to in vitro erythrocyte differentiation were analyzed at d7, d10 and d14 for erythrocyte lineage-specific markers using a Cytoflex flow cytometer. Edited and non-edited cells were analyzed by flow cytometry using the following antibodies: hCD45 V450 (HI30; BD Biosciences), CD34 APC (561; BioLegend), CD71 PE-Cy7 (OKT9; Affymetrix), and CD235a PE (GPA)(GA-R2; BD Biosciences) and a live/dead amino-reactive stain (Invitrogen™ LIVE/DEAD™ Fixable Yellow Dead Cell Stain). Red cell progenitors were gated for single cells, live cells, CD34−/CD45−, and CD71+/CD235a+ cells.

### Hemoglobin Tetramer Analysis Via Cation-Exchange HPLC.

After day 10, HSPCs were further differentiated in tertiary differentiation medium consisting of SFEMII supplemented with 3 U/mL erythropoietin (Peprotech), 200 μg/mL transferrin (Sigma-Aldrich) and 3% human AB serum (Sigma Aldrich) until day 14 before being subjected to HPLC analysis. At day 14 red blood cell pellets were flash frozen post differentiation until tetramer analysis where pellets were then thawed, lysed with 3 times volume of water, vortexed and incubated for 15 min. Cells were then centrifuged for 5 min at 13,000 rpm and supernatant used for input to analyze steady-state hemoglobin tetramer levels. HPLC analysis of hemoglobins in their native form were analyzed on a weak cation-exchange PolyCAT A column (100×4.6-mm, 3 μm, 1,000 Å) (PolyLC Inc.) using a Agilent HPLC system at room temperature. Mobile phase A consists of 20 mM Bis-tris+2 mM KCN, pH 6.96. Mobile phase B consists of 20 mM Bis-tris+2 mM KCN+200 mM NaCl, pH 6.55. Clear hemolysate was diluted four times in buffer A, and then 35 μL was injected onto the column. A flow rate of 1.5 mL/min and the following gradients were used in time (min)/% B organic solvent: (0/10%; 8/40%; 17/90%; 20/10%; 30/stop).

### Hemoglobin Tetramer Analysis Via Reverse Phase HPLC.

Red blood cell pellets were flash frozen post differentiation until tetramer analysis. Pellets were then thawed, lysed with 3 times volume of water, vortexed and incubated for 15 min. Cells were then centrifuged for 5 min at 13,000 rpm and supernatant used for input to analyze steady-state hemoglobin tetramer levels. The chromatographic column was an Aeris™ 3.6 μm WIDEPORE XB-C18 200 Å, LC Column 250×4.6 mm behind a securityGuard™ ULTRA cartridge (Phenomenex). Globin chains were separated using a gradient program of 41-47% solvent B (acetonitrile) mixing with solvent A (0.1% trifluoroacetic acid in HPLC grade water at pH 2.9) and quantified by the area under the curve of the corresponding peaks in reverse-phase HPLC chromatogram.

Allelic Targeting Analysis by ddPCR

2-4d post gene editing, HSPCs were harvested and gDNA extracted using a Qiagen gDNA extraction Kit. gDNA was then digested using HindIII-HF as per manufacturer's instructions (New England Biolabs). The percentage of targeted alleles within a cell population was measured by ddPCR using the following reaction mixture: 2 μL of digested gDNA input, 6.25 μL ddPCR Multiplex SuperMix for Probes (Bio-Rad), primer/probes (1:4 ratio; Integrated DNA Technologies, Coralville, Iowa, USA), volume up to 25 μL with H2O. ddPCR droplets were then generated using an automated droplet generator (Bio-Rad). Thermocycler settings were as follows: 1. 95° C. (10 min), 2. 95° C. (30s), 3. 60° C. (45s, 1C/s ramp rate), 4. 72° C. (3 min) (return to step 2×35 cycles), 5. 98° C. (5 min). Analysis of droplet samples was done using the QX200 Droplet Digital PCR System (Bio-Rad). To determine percentage of alleles targeted, the number of Poisson-corrected integrant copies/mL were divided by the number of Poisson-corrected reference DNA copies/mL.

### Results:

Sickle cell disease is caused by a single nucleotide mutation (adenine to thymine), which changes an amino acid encoded at codon 6 of the HBB gene from glutamic acid (E) to valine (V), resulting in production of hemoglobin S protein (HbS). Production of HbS instead of the WT HbA results in formation of defective hemoglobin tetramers that polymerize upon deoxygenation. Hemoglobin polymerization causes affected red blood cells (RBCs) to lose normal deformability and adopt the archetypal sickle shape. See, e.g., Hoban et al., Blood, 2016 Feb. 18; 127(7):839-48. High-efficiency HDR has been previously demonstrated for knock-in of short donor sequences, for example, a corrective SNP sequence that can revert the E6V mutation back to the wild-type codon in HBB. See e.g., Dever, et al., Nature. 2016 Nov. 17; 539(7629): 384-389. However, correction of alleles containing multiple mutations throughout the gene, for example, as seen in beta-thalassemia major, requires longer donor sequences which may be prone to lower HDR rates and thus lower levels of protein production from corrected alleles. To assess how the AAV6 HBB^{div} donor constructs described above compare to shorter SNP donors in terms of HDR rates and expression levels, a series of constructs were designed to introduce the E6V mutation into the HBB locus in wild-type CD34+ HSPCs as a way to distinguish the HBB protein produced from the HDR allele (forming HbS) from the HBB protein produced from the WT allele (forming HbA). Each construct was designed to include a short 19-nucleotide sequence (SEQ ID NO: 38) which, upon editing of the target HBB allele, introduces the E6V mutation into exon 1 as well as synonymous mutations to the PAM and the sgRNA target site to prevent re-cutting of the edited allele by Cas9. A control construct was designed to knock-in only this short sequence, while test constructs were designed to introduce this sequence in the context of diverged HBB exon sequences and intron sequences from HBG2, HBD and monkey (described in Example 1), respectively. The designs of these constructs are summarized in Table 3 below.

TABLE 3
AAV6 HBB-E6V donor contructs containing heterologous introns

| | | | 3′ regulatory | Homology | |
|---|---|---|---|---|---|
| Construct name | HBB exons | Introns | sequence | arms | FIG. |
| β-SCD-SNP | partial exon 1 | none | none | HBB 5′ & | 6A |
| (control) | containing E6V | | | ex2 | (left) |
| | SNP | | | SEQ ID | |
| | (SEQ ID NO: 38) | | | NOs: 39-40 | |
| β-SCD-HBB^{div}-_ | Diverged HBB | none | bGH | HBB 5′ & | 6A |
| NoIntrons-bGH | exons containing | | (SEQ ID NO: | int1 | (right) |
| | E6V SNP | | 32) | (SEQ ID | |
| | (SEQ ID NO: 41) | | | NOs: 19-20) | |
| β-SCD- | Diverged HBB | HBG2 | bGH | HBB 5′ & | 6A |
| HBB^{div}HBG2^{intr}- | exons containing | (SEQ ID | (SEQ ID NO: | int1 | (right) |
| bGH | E6V SNP | NOs: 11- | 32) | (SEQ ID | |
| | (SEQ ID NO: 41) | 12) | | NOs: 19-20) | |
| β-SCD- | Diverged HBB | HBD | (SEQ ID NO: | HBB 5′ & | 6A |
| HBB^{div}HBD^{intr}-bGH | exons containing | (SEQ ID | 32) | int1 | (right) |
| | E6V SNP | NOs: 13- | | (SEQ ID | |
| | (SEQ ID NO: 41) | 14) | | NOs: 19-20) | |
| β-SCD- | Diverged HBB | Monkey | bGH | HBB 5′ & | 6A |
| HBB^{div}monkey^{intr}- | exons containing | (SEQ ID | (SEQ ID NO: | int1 | (right) |
| bGH | E6V SNP | NOs: 15- | 32) | (SEQ ID | |
| | (SEQ ID NO: 41) | 16) | | NOs: 19-20) | |

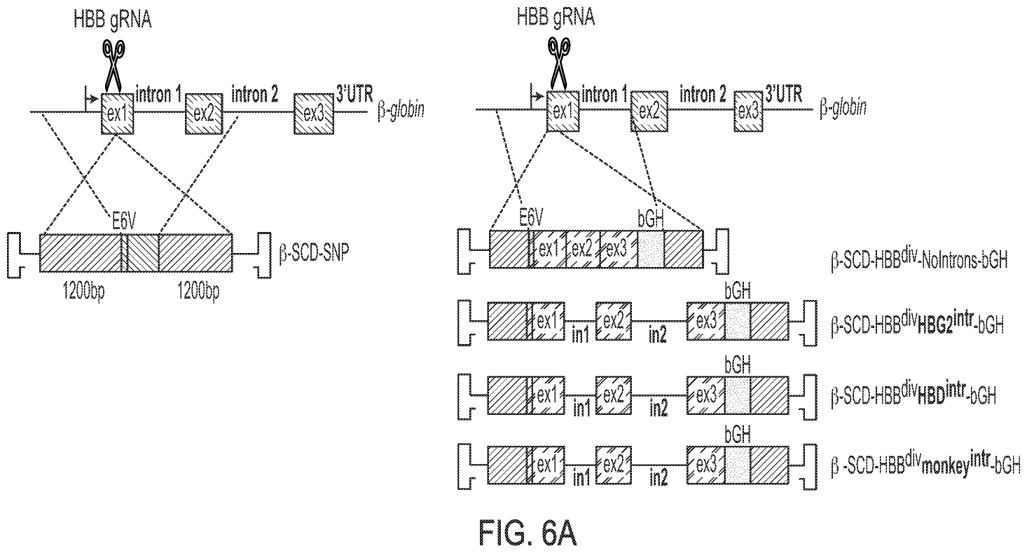

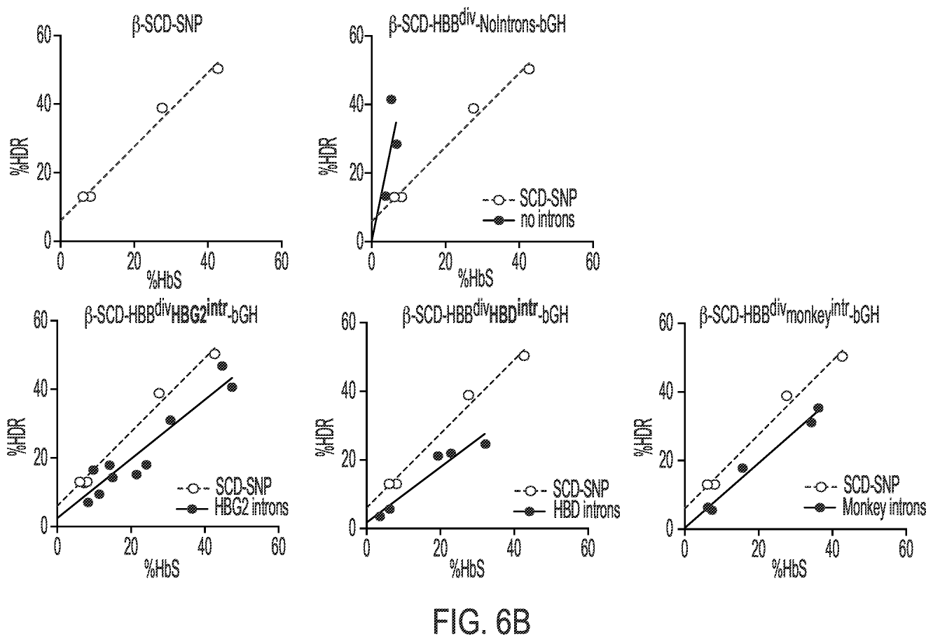

Following genome editing of CD34+ HSPCs utilizing the above AAV6 constructs (and an sgRNA targeting HBB exon 1 (SEQ ID NO: 27)), edited HSPCs underwent erythroid differentiation for fourteen days. HDR rates were assessed by ddPCR and expression from edited alleles containing the E6V mutation was assessed by quantifying the levels of HbS protein by HPLC. As shown in FIG. 6B, the control short-sequence donor converted about 15-50% of WT alleles to the E6V allele, with expression of HbS protein corresponding closely with the rate of HDR (range of about 10-45%). For AAV6 donor constructs containing diverged full-length HBB (HBB^{div}) coding sequences but no introns, similar HDR rates were observed as for the control construct (range of about 15-40%), though very low levels of HbS protein (range of about 5%-10%) were observed. For each of the HBB^{div} constructs containing heterologous introns, HDR rates were again similar to those for the control construct (15-50%) but HbS protein levels approached levels obtained with the short sequence control construct. Constructs containing HBG2 introns demonstrated the highest level HbS production (range of about 10-55%). These results highlight the importance of heterologous intron sequences for the expression of HBB^{div}, and further support the utilization of donor constructs which combine diverged coding sequences with heterologous introns to produce efficient levels of HDR and protein expression, similar to those obtained with short SNP donor sequences.

### Example 5: Optimized Poly A Sequences and HBG2 Intron Sequences Improves HDR Rates and Protein Expression

### Optimization of Poly A Sequences

To assess whether protein production from alleles edited with HBB^{div} donor constructs could be further improved, a series of donor constructs comprising HBG2 intron sequences and diverged HBB exon sequences linked to T2A-EGFP were generated to test an array of polyadenylation signal sequences, including those from the following genes: bovine Growth Hormone (bGH), Hemoglobin Subunit Epsilon 1 (HBE1), Hemoglobin Subunit Gamma 2 (HBG2), Hemoglobin Subunit Gamma 1 (HBG1), Hemoglobin Subunit Delta (HBD), Hemoglobin Subunit Zeta (HBZ), Hemoglobin Subunit Alpha 2 (HBA2), Hemoglobin Subunit Alpha 1 (HBA1), Human growth hormone (hGH), rabbit beta globin (RbGlob), a synthetic poly A sequence based on rabbit beta globin poly A (SynthRbGlob) (Levitt et al., Genes Dev. 1989 Jul.; 3(7):1019-25), and Simian Virus 40 (SV40). The designs of these constructs are summarized in Table 4 below.

TABLE 4
HBB^{div}-HBG2^{intr} donor constructs containing alternate poly A sequences

| | HBB | | | | |
|---|---|---|---|---|---|
| | diverged | | 3′ regulatory | Homology | |
| Construct name | exons | Introns | sequence | arms | FIG. |
| HBB-2A-EGFP | none | none | none | SEQ ID | 3A |
| | | | | NOs: 21-22 | |
| β-HBB^{div}-EGFP- | SEQ ID | none | bGH poly A | HBB 5′ & | 7A |
| bGH | NOs: 35- | | (SEQ ID NO: | int1 | |
| | 37 | | 32) | (SEQ ID | |
| | | | | NOs: 19-20) | |
| β-HBB^{div}HBG2^{intr}- | SEQ ID | HBG2 int1 & int2 | bGH poly A | HBB 5′ & | 7A |
| EGFP-bGH | NOs: 35- | (SEQ ID NOs: | (SEQ ID NO: | int1 | |
| | 37 | 11-12) | 32) | (SEQ ID | |
| | | | | NOs: 19-20) | |
| β-HBB^{div}HBG2^{intr}- | SEQ ID | HBG2 int1 & int2 | HBEI poly A | HBB 5′ & | 7A |
| EGFP-HBE1 | NOs: 35- | (SEQ ID NOs: | (SEQ ID NO: | int1 | |
| | 37 | 11-12) | 45) | (SEQ ID | |
| | | | | NOs: 19-20) | |
| β-HBB^{div}HBG2^{intr}-_ | SEQ ID | HBG2 int1 & int2 | HBG2 poly A | HBB 5′ & | 7A |
| EGFP-HBG2 | NOs: 35- | (SEQ ID NOs: | (SEQ ID NO: | int1 | |
| | 37 | 11-12) | 46) | (SEQ ID | |
| | | | | NOs: 19-20) | |
| β-HBB^{div}HBG2^{intr}- | SEQ ID | HBG2 int1 & int2 | HBG1 poly A | HBB 5′ & | 7A |
| EGFP-HBG1 | NOs: 35- | (SEQ ID NOs: | (SEQ ID NO: | int1 | |
| | 37 | 11-12) | 47) | (SEQ ID | |
| | | | | NOs: 19-20) | |
| β-HBB^{div}HBG2^{intr}-_ | SEQ ID | HBG2 int1 & int2 | HBD poly A | HBB 5′ & | 7A |
| EGFP-HBD | NOs: 35- | (SEQ ID NOs: | (SEQ ID NO: | int1 | |
| | 37 | 11-12) | 48) | (SEQ ID | |
| | | | | NOs: 19-20) | |
| β-HBB^{div}HBG2^{intr}- | SEQ ID | HBG2 int1 & int2 | HBZ poly A | HBB 5′ & | 7A |
| EGFP-HBZ | NOs: 35- | (SEQ ID NOs: | (SEQ ID NO: | int1 | |
| | 37 | 11-12) | 49) | (SEQ ID | |
| | | | | NOs: 19-20) | |
| β-HBB^{div}HBG2^{intr}- | SEQ ID | HBG2 int1 & int2 | HBA2 poly A | HBB 5′ & | 7A |
| EGFP-HBA2 | NOs: 35- | (SEQ ID NOs: | (SEQ ID NO: | int1 | |
| | 37 | 11-12) | 50) | (SEQ ID | |
| | | | | NOs: 19-20) | |
| β-HBB^{div}HBG2^{intr}- | SEQ ID | HBG2 int1 & int2 | HBA1 poly A | HBB 5′ & | 7A |
| EGFP-HBA1 | NOs: 35- | (SEQ ID NOs: | (SEQ ID NO: | int1 | |
| | 37 | 11-12) | 51) | (SEQ ID | |
| | | | | NOs: 19-20) | |
| β-HBB^{div}HBG2^{intr}- | SEQ ID | HBG2 int1 & int2 | hGH poly A | HBB 5′ & | 7A |
| EGFP-hGH | NOs: 35- | (SEQ ID NOs: | (SEQ ID NO: | int1 | |
| | 37 | 11-12) | 52) | (SEQ ID | |
| | | | | NOs: 19-20) | |
| β-HBB^{div}HBG2^{intr}- | SEQ ID | HBG2 int1 & int2 | RbGlob poly A | HBB 5′ & | 7A |
| EGFP-RbGlob | NOs: 35- | (SEQ ID NOs: | (SEQ ID NO: | int1 | |
| | 37 | 11-12) | 53) | (SEQ ID | |
| | | | | NOs: 19-20) | |
| β-HBB^{div}HBG2^{intr}- | SEQ ID | HBG2 int1 & int2 | SynthRbGlob | HBB 5′ & | 7A |
| EGFP- | NOs: 35- | (SEQ ID NOs: | poly A | int1 | |
| SynthRbGlob | 37 | 11-12) | (SEQ ID NO: | (SEQ ID | |
| | | | 54) | NOs: 19-20) | |
| β-HBB^{div}HBG2^{intr}- | SEQ ID | HBG2 int1 & int2 | SV40 poly A | HBB 5′ & | 7A |
| EGFP-SV40 | NOs: 35- | (SEQ ID NOs: | (SEQ ID NO: | int1 | |
| | 37 | 11-12) | 55) | (SEQ ID | |
| | | | | NOs: 19-20) | |

Following genome editing of CD34+ HSPCs utilizing the above AAV6 constructs (and an sgRNA targeting HBB exon 1 (SEQ ID NO: 27)), edited HSPCs underwent erythroid differentiation for ten days. EGFP expression following knock-in of test constructs was compared to EGFP expression from the endogenous HBB locus tagged with EGFP (representative of physiological HBB expression, as described in Example 1).

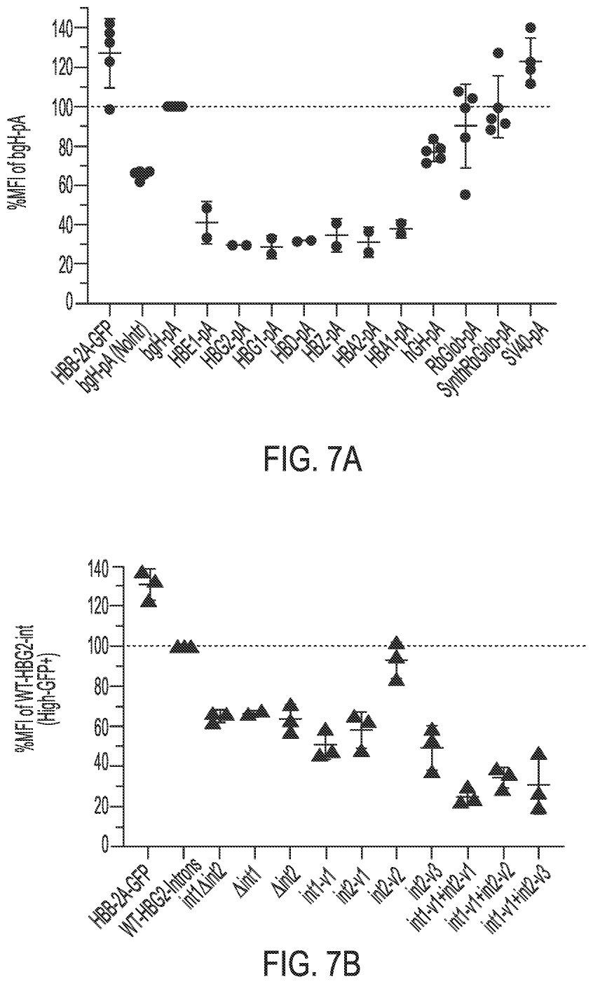

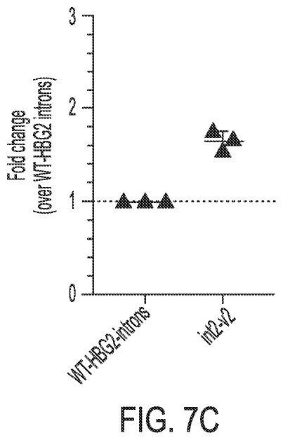

As shown in FIG. 7A, EGFP expression from knock-in of the HBB^{div}HBG2^{intr} test construct containing the bGH poly A sequence was similar to that observed from tagging of the endogenous HBB locus with EGFP. Several additional poly A sequences facilitated EGFP expression approaching or exceeding that observed after knock-in of the HBB^{div}HBG2^{intr} test construct containing the bGH poly A sequence, including hGH, RbGlob, SynthRbGlob and SV40 poly A sequences. Thus, a variety of poly A sequences can be utilized to effectively enhance protein expression from knocked-in HBB^{div}HBG2^{intr} donor sequences.

### Optimization of HBG2 Intron Sequences

An additional series of donor constructs comprising diverged HBB exon sequences linked to T2A-EGFP and bGH poly A were generated to test the impact of modifications to the HBG2 intron sequences on expression levels and HDR rates. The following modifications to HBG2 introns 1 and 2 were tested: (i) Int1-v1: deletion of nucleotides 21-67 of WT intron 1 sequence; (ii) int2-v1: deletion of nucleotides 232-437 and 513-834 of WT intron 2 sequence; (iii) int2-v2: deletion of nucleotides 21-437 and 513-834 of WT intron 2 sequence; and (iv) int2-v3: deletion of nucleotides 161-834 of WT intron 2 sequence.

The designs of HBB^{div}-EGFP-bGH constructs containing these modified intron sequences are summarized in Table 5 below.

TABLE 5
HBB^{div} donor constructs containing modified HBG2 intron sequences

| | HBB | | | | |
|---|---|---|---|---|---|
| | diverged | | 3′ regulatory | Homology | |
| Construct name | exons | Introns | sequence | arms | FIG. |
| HBB-2A-EGFP | none | none | none | HBB int2 & | 3A |
| | | | | 3′ | |
| | | | | (SEQ ID | |
| | | | | NOs: 21-22) | |
| β-HBB^{div}HBG2^{intr}- | SEQ ID | HBG2 int1 & | bGH poly A | HBB 5′ & | 7B |
| EGFP-bGH | NOs: 35-37 | int2 | (SEQ ID NO: | int1 | |
| | | (SEQ ID NOs: | 32) | (SEQ ID | |
| | | 11-12) | | NOs: 19-20) | |
| β-HBB^{div}-EGFP-bGH | SEQ ID | None | bGH poly A | HBB 5′ & | 7B |
| | NOs: 35-37 | (Δint1Δint2) | (SEQ ID NO: | int1 | |
| | | | 32) | (SEQ ID | |
| | | | | NOs: 19-20) | |
| β- | SEQ ID | HBG2 intron 2 | bGH poly A | HBB 5′ & | 7B |
| HBB^{div}HBG2^{intr}Δint1- | NOs: 35-37 | only (SEQ ID | (SEQ ID NO: | int1 | |
| EGFP-bGH | | NO: 12) | 32) | (SEQ ID | |
| | | | | NOs: 19-20) | |
| β- | SEQ ID | HBG2 intron 1 | bGH poly A | HBB 5′ & | 7B |
| HBB^{div}HBG2^{intr}Δint2- | NOs: 35-37 | only (SEQ ID | (SEQ ID NO: | int1 | |
| EGFP-bGH | | NO: 11) | 32) | (SEQ ID | |
| | | | | NOs: 19-20) | |
| β- | SEQ ID | HBG2 intron 1- | bGH poly A | HBB 5′ & | 7B |
| HBB^{div}HBG2^{intr}int1- | NOs: 35-37 | v1 (SEQ ID NO: | (SEQ ID NO: | int1 | |
| v1-EGFP-bGH | | 75) and HBG2 | 32) | (SEQ ID | |
| | | intron 2 (SEQ ID | | NOs: 19-20) | |
| | | NO: 12) | | | |
| β- | SEQ ID | HBG2 intron 1 | bGH poly A | HBB 5′ & | 7B |
| HBB^{div}HBG2^{intr}int2- | NOs: 35-37 | (SEQ ID NO: | (SEQ ID NO: | int1 | |
| v1-EGFP-bGH | | 11) and HBG2 | 32) | (SEQ ID | |
| | | intron 2-v1 (SEQ | | NOs: 19-20) | |
| | | ID NO: 77) | | | |
| β- | SEQ ID | HBG2 intron 1 | bGH poly A | HBB 5′ & | 7B |
| HBB^{div}HBG2^{intr}int2- | NOs: 35-37 | (SEQ ID NO: | (SEQ ID NO: | int1 | |
| v2-EGFP-bGH | | 11) and HBG2 | 32) | (SEQ ID | |
| | | intron 2-v2 (SEQ | | NOs: 19-20) | |
| | | ID NO: 78) | | | |
| β- | SEQ ID | HBG2 intron 1 | bGH poly A | HBB 5′ & | 7B |
| HBB^{div}HBG2^{intr}int2- | NOs: 35-37 | (SEQ ID NO: | (SEQ ID NO: | int1 | |
| v3-EGFP-bGH | | 11) and HBG2 | 32) | (SEQ ID | |
| | | intron 2-v2 (SEQ | | NOs: 19-20) | |
| | | ID NO: 79) | | | |
| β- | SEQ ID | HBG2 intron 1- | bGH poly A | HBB 5′ & | 7B |
| HBB^{div}HBG2^{intr}int1- | NOs: 35-37 | v1 (SEQ ID NO: | (SEQ ID NO: | int1 | |
| v1-int2-v1-EGFP- | | 75) and HBG2 | 32) | (SEQ ID | |
| bGH | | intron 2 v1 (SEQ | | NOs: 19-20) | |
| | | ID NO: 77) | | | |
| β- | SEQ ID | HBG2 intron 1- | bGH poly A | HBB 5′ & | 7B |
| HBB^{div}HBG2^{intr}int1- | NOs: 35-37 | v1 (SEQ ID NO: | (SEQ ID NO: | int1 | |
| v1-int2-v2-EGFP- | | 75) and HBG2 | 32) | (SEQ ID | |
| bGH | | intron 2 v1 (SEQ | | NOs: 19-20) | |
| | | ID NO: 78) | | | |
| β- | SEQ ID | HBG2 intron 1- | bGH poly A | HBB 5′ & | 7B |
| HBB^{div}HBG2^{intr}int1- | NOs: 35-37 | v1 (SEQ ID NO: | (SEQ ID NO: | int1 | |
| v1-int2-v3-EGFP- | | 75) and HBG2 | 32) | (SEQ ID | |
| bGH | | intron 2 v1 (SEQ | | NOs: 19-20) | |
| | | ID NO: 79) | | | |

Following genome editing of CD34+ HSPCs utilizing the above AAV6 constructs (and an sgRNA targeting HBB exon 1 (SEQ ID NO: 27)), edited HSPCs underwent erythroid differentiation for ten days. EGFP expression following knock-in of test constructs was compared to EGFP expression from the endogenous HBB locus tagged with EGFP (representative of physiological HBB expression, as described in Example 1).

As shown in FIG. 7B, EGFP expression from knock-in of the HBB^{div}-EGFP-bGH test construct containing wild-type HBG2 intron 1 and 2 sequences approached the levels observed from tagging of the endogenous HBB locus with EGFP. Modifications that shortened HBG2 intron 1 or intron 2 largely reduced EGFP expression relative to that seen with full length introns. However, the construct containing wild-type HBG2 intron 1 and a deletion of nucleotides 21-437 and 513-834 from intron 2 (HBG2^{i2v2}) facilitated EGFP expression equivalent to that observed with full length introns. Moreover, knock-in efficiency (i.e. HDR rates) of this construct were nearly 2-fold higher compared to that observed for the donor construct containing full-length HBG2 introns (FIG. 7C).

### Example 6: Optimized HBB^{div} HBG2^{intr} Donor Constructs Rescue the SCD Phenotype in Patient-Derived CD34+ HSPCs

Following the optimization of poly A and HBG2 intron sequences, additional HBB^{div} donor constructs containing these sequences were generated to test their ability to rescue the SCD phenotype caused by the E6V mutation at the HBB locus in SCD patient-derived CD34+ HSPCs (provided by Dr. John Tisdale and the U.S. Department of Health and Human Services). Both full-length and shortened HBG2 intron sequences were tested in combination with bGH and SV40 poly A sequences, respectively. The designs of constructs containing these optimized sequences are summarized in Table 6 below.

TABLE 6
HBB^{div} donor constructs containing HBG2 intron sequences

| | HBB | | | | |
|---|---|---|---|---|---|
| | diverged | | 3′ regulatory | Homology | |
| Construct name | exons | Introns | sequence | arms | FIG. |
| β-HBB^{div}HBG2^{intr}- | SEQ ID | HBG2 int1 & int2 | bGH poly A | HBB 5′ & int1 | 8A |
| bGH | NOs: 35- | (SEQ ID NOs: | (SEQ ID NO: | (SEQ ID NOs: | |
| | 37 | 11-12) | 32) | 19-20) | |
| β-HBB^{div}HBG2^{intr}- | SEQ ID | HBG2 int1 & int2 | SV40 poly A | HBB 5′ & int1 | 8A |
| SV40 | NOs: 35- | (SEQ ID NOs: | (SEQ ID NO: | (SEQ ID NOs: | |
| | 37 | 11-12) | 55) | 19-20) | |
| β- | SEQ ID | HBG2 intron 1 | bGH poly A | HBB 5′ & int1 | 8A |
| HBB^{div}HBG2^{intr}int2- | NOs: 35- | (SEQ ID NO: 11) | (SEQ ID NO: | (SEQ ID NOs: | |
| v2-bGH | 37 | and HBG2 intron | 32) | 19-20) | |
| | | 2-v2 (SEQ ID | | | |
| | | NO: 78) | | | |
| β- | SEQ ID | HBG2 intron 1 | SV40 poly A | HBB 5′ & int1 | 8A |
| HBB^{div}HBG2^{intr}int2- | NOs: 35- | (SEQ ID NO: 11) | (SEQ ID NO: | (SEQ ID NOs: | |
| v2-SV40 | 37 | and HBG2 intron | 55) | 19-20) | |
| | | 2-v2 (SEQ ID | | | |
| | | NO: 78) | | | |

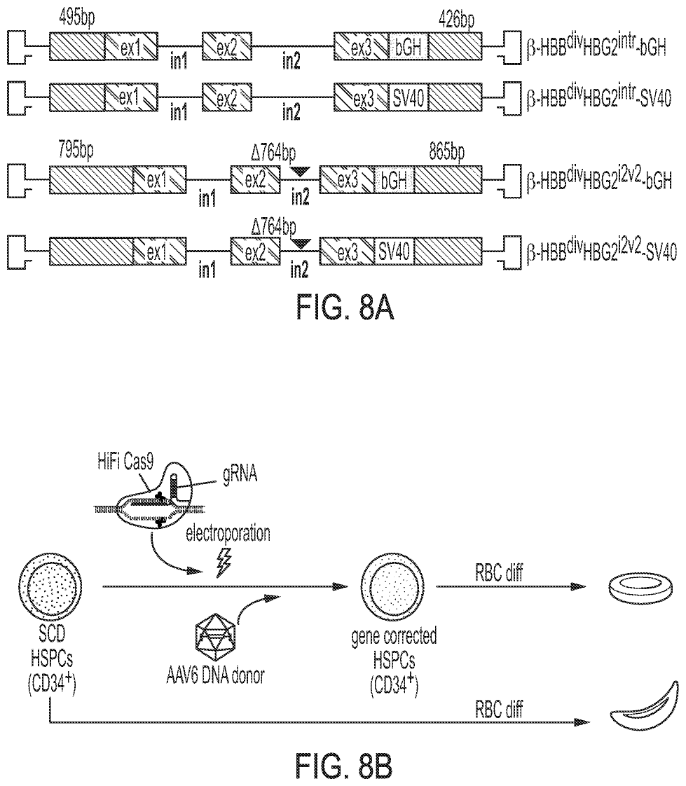

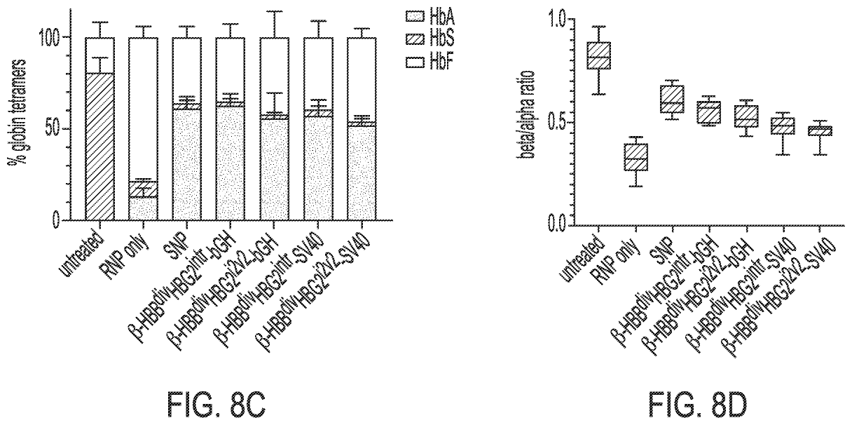

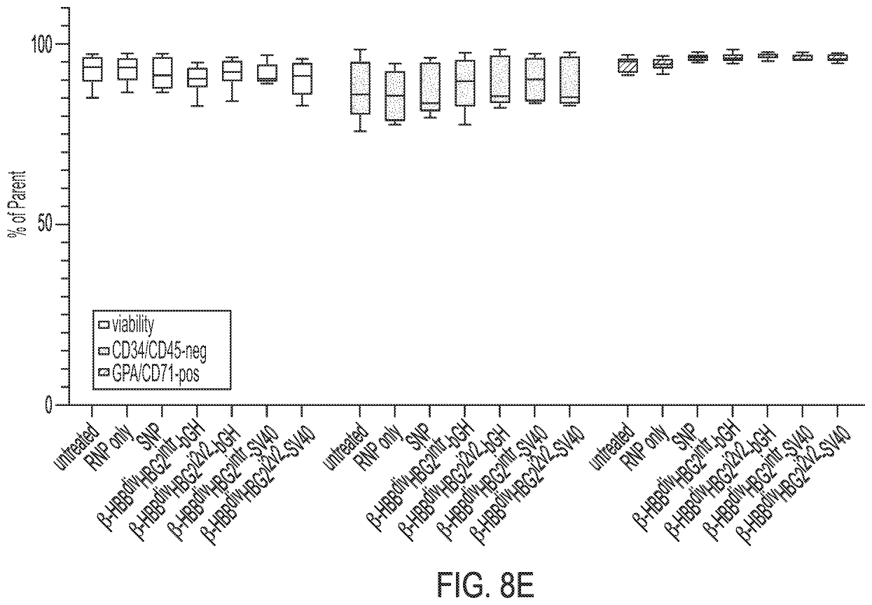

Constructs were targeted for knock-in at the HBB locus using homology arms to exon 1, and gene editing was performed with a guide RNA that generates a cut site within exon 1 (SEQ ID NO: 27). SCD patient-derived CD34+ HSPCs were treated with ribonucleoprotein (RNP) only (pre-complexed HiFi Cas9 and the HBB guide RNA but without donor constructs) as a negative control. As a positive control for HbS to HbA conversion, the HSPCs were edited with RNP and an AAV6 donor containing a corrective SNP sequence (SEQ ID NO: 80) that can revert the E6V mutation back to the wild-type codon in HBB. Both edited and non-edited SCD patient-derived CD34+ HSPCs underwent erythroid differentiation for seven days (FIG. 8B), then assessed for HbA, HbS and HbF formation.

As shown in FIG. 8C, knock-in of constructs containing diverged HBB coding sequences, HBG2 intronic sequences (full length and shortened intron 2 (i2V2)) and bGH or SV40 poly A sequences resulted in robust conversion of sickle hemoglobin (HbS) to normal adult hemoglobin (HbA). HbA production from knock-in of the HBB^{div}HBG^{int} donor sequences was on par with HbA production resulting from knock-in of the short corrective SNP donor.

Beta to alpha chain ratios were also assessed following editing using reverse-phase HPLC (FIG. 8D). While editing with RNPs without donors significantly reduced the production of beta chains (likely due to frameshift mutations in HBB from indel formation), knock-in of each of the four HBB^{div}HBG^{int} donor sequences resulted in beta:alpha globin chain ratios of ≥0.5 (with a ratio of at least 0.5 representing beta-thalassemia trait), similar to the ratios observed with knock-in of the short corrective SNP donor.

Viability and red blood cell differentiation potential of edited patient-derived CD34+ HSPCs were also assessed. Edited and non-edited HSPCs subjected to in vitro erythrocyte differentiation were analyzed at d7, d10 and d14 for viability and the presence of erythrocyte lineage-specific markers. As shown in FIG. 8E, cell viability following editing with each HBB^{div}HBG^{int} donor construct was unaffected when compared with non-edited cells and cells edited with the corrective SNP donor. Red blood cell differentiation potential of edited CD34+ HSPCs was also unaffected, as demonstrated by nearly equal amounts of stem cell marker (CD34/CD45)-negative and erythroid cell marker (GPA/CD71)-positive cells across all edited and non-edited populations following the in vitro differentiation process. In total, these results demonstrate that full length diverged HBB coding sequences combined with heterologous intron sequences can correct or replace mutant HBB alleles in CD34+ HSPCs, leading to rescue of a hemoglobinopathy phenotype while preserving the potential for RBC differentiation.

### Example 7: Inclusion of Heterologous Introns Improves Therapeutic Protein Expression from HBB and HBA1 Loci

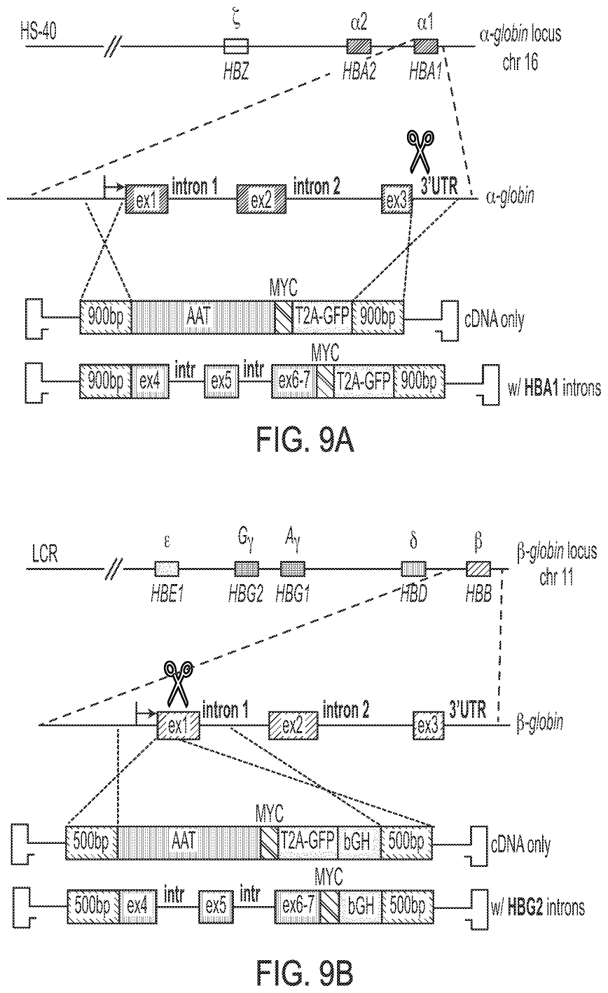

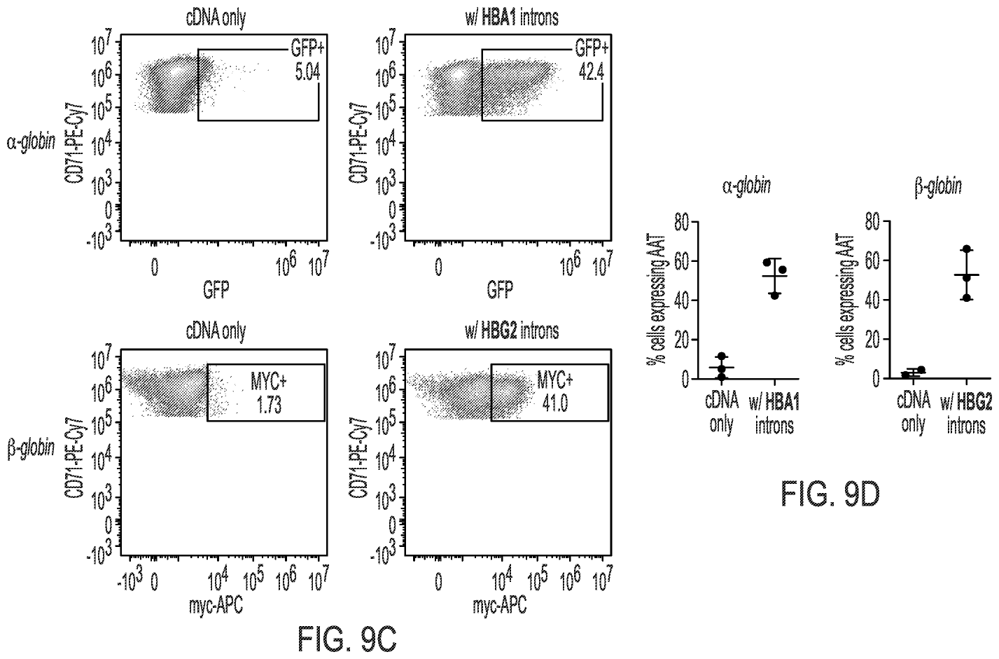

Additional donor constructs were generated and tested to see if inclusion of heterologous intron sequences were necessary for the expression of the therapeutic protein alpha-1 antitrypsin (AAT) following knock-in at two different loci. AAV6 donor constructs were designed to include the AAT coding sequence (exons 4-7; SEQ ID NO:71) fused to a myc tag, without introns or with heterologous introns from HBA1 or HBG2. Donor constructs containing HBA1 introns were designed with homology arms targeting the HBA1 locus (FIG. 9A), while constructs containing HBG2 introns were designed with homology arms targeting HBB (FIG. 9B). The designs of these constructs are summarized in Table 7 below.

TABLE 7
AAT donor constructs containing heterologous intron sequences

| Construct | Coding | | 3′ regulatory | Homology | |
|---|---|---|---|---|---|
| name | sequence | Introns | sequence | arms | FIG. |
| α-AAT-myc- | AAT cDNA | none | none | HBA1 5′ & | 9A |
| T2A-EGFP | (SEQ ID NO: | | | 3′ (SEQ ID | |
| | 71) | | | NOs: 23- | |
| | | | | 24) | |
| α-AAT- | AAT cDNA | HBA1 int1 & int2 | None | HBA1 5′ & | 9A |
| HBA1^{intr}-myc- | (SEQ ID NO: | (SEQ ID NOs: 28- | | 3′ (SEQ ID | |
| T2A-EGFP | 71) | 29) | | NOs: 23- | |
| | | | | 24) | |
| β-AAT-myc- | AAT cDNA | none | bGH poly A | HBB 5′ & | 9B |
| T2A-EGFP-bGH | (SEQ ID NO: | | (SEQ ID NO: | int1 | |
| | 71) | | 32) | (SEQ ID | |
| | | | | NOs: 19- | |
| | | | | 20) | |
| β-AAT- | AAT cDNA | HBG2 int1 & int2 | bGH poly A | HBB 5′ & | 9B |
| HBG2^{intr}-myc- | (SEQ ID NO: | (SEQ ID NOs: 11- | (SEQ ID NO: | int1 | |
| bGH | 71) | 12) | 32) | (SEQ ID | |
| | | | | NOs: 19- | |
| | | | | 20) | |

Knock-in to the HBA1 locus and HBB locus was facilitated by guide RNAs targeting the 3′UTR region of HBA1 (SEQ ID NO: 25), and exon 1 of HBB (SEQ ID NO: 27), respectively. Following gene editing with the above donor constructs, edited CD34+ HSPCs underwent erythroid differentiation for seven days (FIG. 8B), then assessed for AAT expression by way of EGFP expression or by intracellular staining for myc expression.

As shown in FIG. 9C and FIG. 9D, inclusion of heterologous intron sequences enabled robust expression of AAT following knock-in at both the alpha-globin and beta-globin locus, while knock-in of AAT donor sequences without heterologous introns resulted in low to undetectable levels of AAT expression. These results further demonstrate the effectiveness of adding heterologous introns to donor constructs for enabling and improving therapeutic protein expression in HDR-based gene correction and replacement applications.

TABLE 8
Sequence Tables

| SEQ | | |
|---|---|---|
| ID | | |
| NO: | Name | Sequence |
| 1 | Diverged | ATGGTcCAcCTcACcCCcGAaGAaAAaTCcGCaGTcACcGCTCTCT |
| | HBB with | GGGGAAAAGTCAATGTCGACGAGGTGGGAGGCGAAGCTCTC |
| | HBB Introns | GGAAGgttggtatcaaggttacaagacaggtttaaggagaccaatagaaactgggcatgtgga |
| | | gacagagaagactcttgggtttctgataggcactgactctctctgcctattggtctattttcccaccctta |
| | | gACTCCTCGTCGTGTATCCCTGGACACAAAGATTTTTCGAAA |
| | | GCTTCGGCGACCTCAGCACACCCGACGCCGTGATGGGGAAT |
| | | CCCAAAGTCAAAGCCCACGGGAAAAAGGTCCTGGGCGCTTT |
| | | CTCCGACGGACTCGCCCATCTCGATAATCTGAAAGGAACTTT |
| | | CGCTACCCTCTCCGAACTCCATTGCGATAAACTCCATGTCGA |
| | | CCCCGAAAATTTTAGAgtgagtctatgggacgcttgatgttttctttccccttcttttctatg |
| | | gttaagttcatgtcataggaaggggataagtaacagggtacagtttagaatgggaaacagacgaatg |
| | | attgcatcagtgtggaagtctcaggatcgttttagtttcttttatttgctgttcataacaattgttttctt |
| | | ttgtttaattcttgctttctttttttttcttctccgcaatttttactattatacttaatgcctta |
| | | acattgtgtataacaaaaggaaatatctctgagatacattaagtaacttaaaaaaaaactttacacagtc |
| | | tgcctagtacattactatttggaatatatgtgtgcttatttgcatattcataatctccctactttattttcttt |
| | | tatttttaattgatacataatcattatacatatttatgggttaaagtgtaatgttttaatatgtgtacacata |
| | | ttgaccaaatcagggtaattttgcatttgtaattttaaaaaatgctttcttcttttaatatacttttttgtttat |
| | | cttatttctaatactttccctaatctctttctttcagggcaataatgatacaatgtatcatgcctctttgcac |
| | | cattctaaagaataacagtgataatttctgggttaaggcaatagcaatatctctgcatataaatatttctgca |
| | | tataaattgtaactgatgtaagaggtttcatattgctaatagcagctacaatccagctaccattctgcttttatt |
| | | ttatggttgggataaggctggattattctgagtccaagctaggcccttttgctaatcatgttcatac |
| | | ctcttatcttcctcccacagCTGCTCGGGAATGTCCTCGTGTGCGTCCTCGCTCACCATTTCGGGAAGG |
| | | AGTTTACACCCCCCGTCCAAGCCGCTTACCAAAAGGTCGTCGCCGGAGTCGCCAACGCTCTCGCTCAT |
| | | AAATACCAT |
| 2 | HBB (CDS | ATGGTcCAcCTcACcCCcGAaGAaAAaTCcGCaGTcACcGCTCTCT |
| | diverged with | GGGGAAAAGTCAATGTCGACGAGGTGGGAGGCGAAGCTCTC |
| | HBG2 | GGAAGgtaggctctggtgaccaggacaagggagggaaggaaggaccctgtgcctggcaaaa |
| | introns) | gtccaggtcgcttctcaggatttgtggcaccttctgactgtcaaactgttcttgtcaatctcacagACT |
| | | CCTCGTCGTGTATCCCTGGACACAAAGATTTTTCGAAAGCTT |
| | | CGGCGACCTCAGCACACCCGACGCCGTGATGGGGAATCCCA |
| | | AAGTCAAAGCCCACGGGAAAAAGGTCCTGGGCGCTTTCTCC |
| | | GACGGACTCGCCCATCTCGATAATCTGAAAGGAACTTTCGCT |
| | | ACCCTCTCCGAACTCCATTGCGATAAACTCCATGTCGACCCC |
| | | GAAAATTTTAGAgtgagtccaggagatgtttcagcactgttgcctttagtctcgaggcaac |
| | | ttagacaactgagtattgatctgagcacagcagggtgtgagctgtttgaagatactggggttgggagt |
| | | gaagaaactgcagaggactaactgggctgagacccagtggcaatgttttagggcctaaggagtgcc |
| | | tctgaaaatctagatggacaactttgactttgagaaaagagaggtggaaatgaggaaaatgacttttctt |
| | | tattagatttcggtagaaagaactttcacctttcccctatttttgttattcgttttaaaacatctatctggagg |
| | | caggacaagtatggtcattaaaaagatgcaggcagaaggcatatattggctcagtcaaagtggggaa |
| | | ctttggtggccaaacatacattgctaaggctattcctatatcagctggacacatataaaatgctgctaat |
| | | gcttcattacaaacttatatcctttaattccagatgggggcaaagtatgtccaggggtgaggaacaattg |
| | | aaacatttgggctggagtagattttgaaagtcagctctgtgtgtgtgtgtgtgtgtgtgcgcgcgtgtgtt |
| | | tgtgtgtgtgtgagagcgtgtgtttcttttaacgttttcagcctacagcatacagggttcatggtggcaag |
| | | aagataacaagatttaaattatggccagtgactagtgctgcaagaagaacaactacctgcatttaatgg |
| | | gaaagcaaaatctcaggctttgagggaagttaacataggcttgattctgggtggaagcttggtgtgta |
| | | gttatctggaggccaggctggagctctcagctcactatgggttcatctttattgtctcctttcatctcaaca |
| | | gCTGCTCGGGAATGTCCTCGTGTGCGTCCTCGCTCACCATTTC |
| | | GGGAAGGAGTTTACACCCCCCGTCCAAGCCGCTTACCAAAA |
| | | GGTCGTCGCCGGAGTCGCCAACGCTCTCGCTCATAAATACCAT |
| 3 | HBB (CDS | ATGGTcCAcCTcACcCCcGAaGAaAAaTCcGCaGTcACcGCTCTCT |
| | diverged with | GGGGAAAAGTCAATGTCGACGAGGTGGGAGGCGAAGCTCTC |
| | HBD introns) | GGAAGgttggtatcaaggttataagagaggctcaaggaggcaaatggaaactgggcatgtgta |
| | | gacagagaagactcttgggtttctgataggcactgactctctgtcccttgggctgttttcctaccctcag |
| | | ACTCCTCGTCGTGTATCCCTGGACACAAAGATTTTTCGAAAG |
| | | CTTCGGCGACCTCAGCACACCCGACGCCGTGATGGGGAATC |
| | | CCAAAGTCAAAGCCCACGGGAAAAAGGTCCTGGGCGCTTTC |
| | | TCCGACGGACTCGCCCATCTCGATAATCTGAAAGGAACTTTC |
| | | GCTACCCTCTCCGAACTCCATTGCGATAAACTCCATGTCGAC |
| | | CCCGAAAATTTTAGAgtgagtccaggagatgcttcacttttctctttttactttctaatcttacatttt |
| | | ggttcttttacctacctgctcttctcccacatttttgtcattttactatattttatcatttaatgcttctaaa |
| | | attttgttaattttttatttaaatattctgcattttttccttcctcacaatcttgctattttaaattatt |
| | | taatatcctgtctttctctcccaaccccctcccttcatttttccttctctaacaacaactcaaattatgc |
| | | ataccagctctcacctgctaattctgcacttagaataatccttttgtctctccacatgggtatgggagaggc |
| | | tccaactcaaagatgagaggcatagaatactgttttagaggctataaatcattttacaataaggaataa |
| | | ttggaattttataaattctgtagtaaatggaatggaaaggaaagtgaatatttgattatgaaagactaggc |
| | | agttacactggaggtggggcagaagtcgttgctaggagacagcccatcatcacactgattaatcaattaatttg |
| | | tatctattaatctgtttatagtaattaatttgtatatgctatatacacatacaaaattaaaactaatttggaatt |
| | | aatttgtatatagtattatacagcatatatagcatatatgtacatatatagactacatgctagttaagtaca |
| | | tagaggatgtgtgtgtatagatatatgttatatgtatgcattcatatatgtacttatttatgctgatggg |
| | | aataacctggggatcagttttgtctaagatttgggcagaaaaaaatgggtgttggctcagtttctcagaag |
| | | ccagtctttatttctctgttaaccatatgcatgtatctgcctacctcttctccgcagCTGCTCGGGAATGT |
| | | CCTCGTGTGCGTCCTCGCTCACCATTTCGGGAAGGAGTTTAC |
| | | ACCCCCCGTCCAAGCCGCTTACCAAAAGGTCGTCGCCGGAG |
| | | TCGCCAACGCTCTCGCTCATAAATACCAT |
| 4 | HBB (CDS | ATGGTcCAcCTcACcCCcGAaGAaAAaTCcGCaGTcACcGCTCTCT |
| | diverged with | GGGGAAAAGTCAATGTCGACGAGGTGGGAGGCGAAGCTCTC |
| | HBG-D | GGAAGgtaggctctggtgaccaggacaagggagggaaggaaggaccctgtgcctggcaaaa |
| | hybrid | gtccaggtcgcttctcaggatttgtggcaccttctgactgtcaaactgttcttgtcaatctcacagACT |
| | introns) | CCTCGTCGTGTATCCCTGGACACAAAGATTTTTCGAAAGCTT |
| | | CGGCGACCTCAGCACACCCGACGCCGTGATGGGGAATCCCA |
| | | AAGTCAAAGCCCACGGGAAAAAGGTCCTGGGCGCTTTCTCC |
| | | GACGGACTCGCCCATCTCGATAATCTGAAAGGAACTTTCGCT |
| | | ACCCTCTCCGAACTCCATTGCGATAAACTCCATGTCGACCCCGAAAAT |
| | | TTTAGAgtgagtccaggagatgcttcacttttctctttttactttctaatcttacattttggttcttttacc |
| | | tacctgctcttctcccacatttttgtcattttactatattttatcatttaatgcttctaaaattttgttaat |
| | | tttttatttaaatattctgcattttttccttcctcacaatcttgctattttaaattatttaatatcctgtctt |
| | | tctctcccaaccccctcccttcatttttccttctctaacaacaactcaaattatgcataccagctctcacctg |
| | | ctaattctgcacttagaataatccttttgtctctccacatgggtatgggagaggctccaactcaaagatg |
| | | agaggcatagaatactgttttagaggctataaatcattttacaataaggaataattggaattttataaattc |
| | | tgtagtaaatggaatggaaaggaaagtgaatatttgattatgaaagactaggcagttacactggaggt |
| | | ggggcagaagtcgttgctaggagacagcccatcatcacactgattaatcaattaatttgtatctattaat |
| | | ctgtttatagtaattaatttgtatatgctatatacacatacaaaattaaaactaatttggaattaatttgtatat |
| | | agtattatacagcatatatagcatatatgtacatatatagactacatgctagttaagtacatagaggatgt |
| | | gtgtgtatagatatatgttatatgtatgcattcatatatgtacttatttatgctgatgggaataacctgggga |
| | | tcagttttgtctaagatttgggcagaaaaaaatgggtgttggctcagtttctcagaagccagtctttatttc |
| | | tctgttaaccatatgcatgtatctgcctacctcttctccgcagCTGCTCGGGAATGTCCT |
| | | CGTGTGCGTCCTCGCTCACCATTTCGGGAAGGAGTTTACACC |
| | | CCCCGTCCAAGCCGCTTACCAAAAGGTCGTCGCCGGAGTCG |
| | | CCAACGCTCTCGCTCATAAATACCAT |
| 5 | Div HBB | ATGGTcCAcCTcACcCCcGAaGAaAAaTCcGCaGTcACcGCTCTCT |
| | (with monkey | GGGGAAAAGTCAATGTCGACGAGGTGGGAGGCGAAGCTCTC |
| | introns) | GGAAGgttggtatcaatgttataagagaggctcatggaggtaaatggaagctgggcatgtgtag |
| | | acagagaagactctggaggttctgatagtcattgattctctctgtcccttgggctgttttcctaccctcag |
| | | ACTCCTCGTCGTGTATCCCTGGACACAAAGATTTTTCGAAAG |
| | | CTTCGGCGACCTCAGCACACCCGACGCCGTGATGGGGAATC |
| | | CCAAAGTCAAAGCCCACGGGAAAAAGGTCCTGGGCGCTTTC |
| | | TCCGACGGACTCGCCCATCTCGATAATCTGAAAGGAACTTTC |
| | | GCTACCCTCTCCGAACTCCATTGCGATAAACTCCATGTCGACCCCGAAAATT |
| | | TTAGAgtgagtccaggagatgcttcacttttctctgtttactgtctaatcttacattttagtttttacc |
| | | tacctgctcttcccccacatttttgtcattttactatattttatcatttaatgcttctaaaattttgttattt |
| | | ttttatttaaatattctgcattttttccttcctcacaatcttgctattttaaattatttaatatcctgt |
| | | cctctcctccccaaccccttcccttcgttttcttctctaaccacaactcaaattatgcatgccagctctcac |
| | | gtgctaattctgcacttagaataattctttgtctctccacatgggtatgagagaggctccagctcaaaga |
| | | cgagaggcatagaatactgttttagaggctataaattattttacaataaggaataattggaattttataaat |
| | | ttggtagtaaatgggatggaaaggaaagtgaatatttgattatgaaagactagaaagttacactggag |
| | | gtggggcagaagtcgttgctaggagacagcccatcatcacactgattaatgaattaatttgtatctatta |
| | | atctgtttagagtaattaatttgtatatgctatatacacatacaaaattaaaactaatttggaattaatttgtat |
| | | atagcattatacagcatatatagcatatatgtacatatatagactatatgctagttaagtacacagaggat |
| | | gtgtgtgtatagatatatgttatatgcatgcattcatatatgtacttatttatgctgatgggaataacctggg |
| | | gatcagttttgtctaagatttgcgcagaaaaaaatgggtgttggcccagtttctcagaagccaatctttat |
| | | ttctctgttaaccatatgcatatatctgcctaccttttctctgcagCTGCTCGGGAATGTCC |
| | | TCGTGTGCGTCCTCGCTCACCATTTCGGGAAGGAGTTTACAC |
| | | CCCCCGTCCAAGCCGCTTACCAAAAGGTCGTCGCCGGAGTC |
| | | GCCAACGCTCTCGCTCATAAATACCAT |
| 6 | HBB WT | ATGGTGCATCTGACTCCTGAGGAGAAGTCTGCCGTTACTGCC |
| | | CTGTGGGGCAAGGTGAACGTGGATGAAGTTGGTGGTGAGGC |
| | | CCTGGGCAGgttggtatcaaggttacaagacaggtttaaggagaccaatagaaactgggcat |
| | | gtggagacagagaagactcttgggtttctgataggcactgactctctctgcctattggtctattttcccac |
| | | ccttagGCTGCTGGTGGTCTACCCTTGGACCCAGAGGTTCTTTGA |
| | | GTCCTTTGGGGATCTGTCCACTCCTGATGCTGTTATGGGCAA |
| | | CCCTAAGGTGAAGGCTCATGGCAAGAAAGTGCTCGGTGCCT |
| | | TTAGTGATGGCCTGGCTCACCTGGACAACCTCAAGGGCACCT |
| | | TTGCCACACTGAGTGAGCTGCACTGTGACAAGCTGCACGTG |
| | | GATCCTGAGAACTTCAGGgtgagtctatgggacgcttgatgttttctttccccttcttttc |
| | | tatggttaagttcatgtcataggaaggggataagtaacagggtacagtttagaatgggaaacagacg |
| | | aatgattgcatcagtgtggaagtctcaggatcgttttagtttcttttatttgctgttcataacaattgt |
| | | tttcttttgtttaattcttgctttctttttttttcttctccgcaatttttactattatactta |
| | | atgccttaacattgtgtataacaaaaggaaatatctctgagatacattaagtaacttaaaaaaaaac |
| | | tttacacagtctgcctagtacattactatttggaatatatgtgtgcttatttgcatattcataatct |
| | | ccctactttattttcttttatttttaattgatacataatcattatacatatttatgggttaaagt |
| | | gtaatgttttaatatgtgtacacatattgaccaaatcagggtaattttgcatttgtaattttaaaa |
| | | aatgctttcttcttttaatatacttttttgtttatcttatttctaatactttccctaatctc |
| | | tttctttcagggcaataatgatacaatgtatcatgcctctttgcaccattctaaagaataacagtgataattt |
| | | ctgggttaaggcaatagcaatatctctgcatataaatatttctgcatataaattgtaactgatgtaagagg |
| | | tttcatattgctaatagcagctacaatccagctaccattctgcttttattttatggttgggataaggctggat |
| | | tattctgagtccaagctaggcccttttgctaatcatgttcatacctcttatcttcctcccacagCTCCT |
| | | GGGCAACGTGCTGGTCTGTGTGCTGGCCCATCACTTTGGCAA |
| | | AGAATTCACCCCACCAGTGCAGGCTGCCTATCAGAAAGTGG |
| | | TGGCTGGTGTGGCTAATGCCCTGGCCCACAAGTATCAC |
| 7 | WTHBB | ATGGTGCATCTGACTCCTGAGGAGAAGTCTGCCGTTACTGCC |
| | cDNA | CTGTGGGGCAAGGTGAACGTGGATGAAGTTGGTGGTGAGGC |
| | | CCTGGGCAGGCTGCTGGTGGTCTACCCTTGGACCCAGAGGTT |
| | | CTTTGAGTCCTTTGGGGATCTGTCCACTCCTGATGCTGTTATG |
| | | GGCAACCCTAAGGTGAAGGCTCATGGCAAGAAAGTGCTCGG |
| | | TGCCTTTAGTGATGGCCTGGCTCACCTGGACAACCTCAAGGG |
| | | CACCTTTGCCACACTGAGTGAGCTGCACTGTGACAAGCTGCA |
| | | CGTGGATCCTGAGAACTTCAGG |
| | | CTCCTGGGCAACGTGCTGGTCTGTGTGCTGGCCCATCACTTT |
| | | GGCAAAGAATTCACCCCACCAGTGCAGGCTGCCTATCAGAA |
| | | AGTGGTGGCTGGTGTGGCTAATGCCCTGGCCCACAAGTATCAC |
| 8 | Diverged | ATGGTcCAcCTcACcCCcGAaGAaAAaTCcGCaGTcACcGCTCTCT |
| | HBB cDNA | GGGGAAAAGTCAATGTCGACGAGGTGGGAGGCGAAGCTCTC |
| | | GGAAGACTCCTCGTCGTGTATCCCTGGACACAAAGATTTTTC |
| | | GAAAGCTTCGGCGACCTCAGCACACCCGACGCCGTGATGGG |
| | | GAATCCCAAAGTCAAAGCCCACGGGAAAAAGGTCCTGGGCG |
| | | CTTTCTCCGACGGACTCGCCCATCTCGATAATCTGAAAGGAA |
| | | CTTTCGCTACCCTCTCCGAACTCCATTGCGATAAACTCCATG |
| | | TCGACCCCGAAAATTTTAGA |
| | | CTGCTCGGGAATGTCCTCGTGTGCGTCCTCGCTCACCATTTC |
| | | GGGAAGGAGTTTACACCCCCCGTCCAAGCCGCTTACCAAAA |
| | | GGTCGTCGCCGGAGTCGCCAACGCTCTCGCTCATAAATACCA |
| | | T |
| 9 | HBB Intron 1 | gttggtatcaaggttacaagacaggtttaaggagaccaatagaaactgggcatgtggagacagaga |
| | | agactcttgggtttctgataggcactgactctctctgcctattggtctattttcccacccttag |
| 10 | HBB Intron 2 | gtgagtctatgggacgcttgatgttttctttccccttcttttctatggttaagttcatgtcataggaagggga |
| | | taagtaacagggtacagtttagaatgggaaacagacgaatgattgcatcagtgtggaagtctcaggatcgttt |
| | | tagtttcttttatttgctgttcataacaattgttttcttttgtttaattcttgctttctttttttttcttctccgca |
| | | atttttactattatacttaatgccttaacattgtgtataacaaaaggaaatatctctgagatacattaagtaa |
| | | cttaaaaaaaaactttacacagtctgcctagtacattactatttggaatatatgtgtgcttatttgcatattca |
| | | taatctccctactttattttcttttatttttaattgatacataatcattatacatatttatgggttaaagtgtaatgt |
| | | tttaatatgtgtacacatattgaccaaatcagggtaattttgcatttgtaattttaaaaaatgctttcttctttta |
| | | atatacttttttgtttatcttatttctaatactttccctaatctctttctttcagggcaataatgatacaatgtatc |
| | | atgcctctttgcaccattctaaagaataacagtgataatttctgggttaaggcaatagcaatatctctgca |
| | | tataaatatttctgcatataaattgtaactgatgtaagaggtttcatattgctaatagcagctacaatccag |
| | | ctaccattctgcttttattttatggttgggataaggctggattattctgagtccaagctaggcccttttgcta |
| | | atcatgttcatacctcttatcttcctcccacag |
| 11 | HBG2 intron 1 | gtaggctctggtgaccaggacaagggagggaaggaaggaccctgtgcctggcaaaagtccaggt |
| | | cgcttctcaggatttgtggcaccttctgactgtcaaactgttcttgtcaatctcacag |
| 12 | HBG2 intron 2 | gtgagtccaggagatgtttcagcactgttgcctttagtctcgaggcaacttagacaactgagtattgatc |
| | | tgagcacagcagggtgtgagctgtttgaagatactggggttgggagtgaagaaactgcagaggact |
| | | aactgggctgagacccagtggcaatgttttagggcctaaggagtgcctctgaaaatctagatggaca |
| | | actttgactttgagaaaagagaggtggaaatgaggaaaatgacttttctttattagatttcggtagaaag |
| | | aactttcacctttcccctatttttgttattcgttttaaaacatctatctggaggcaggacaagtatggtcatta |
| | | aaaagatgcaggcagaaggcatatattggctcagtcaaagtggggaactttggtggccaaacataca |
| | | ttgctaaggctattcctatatcagctggacacatataaaatgctgctaatgcttcattacaaacttatatcc |
| | | tttaattccagatgggggcaaagtatgtccaggggtgaggaacaattgaaacatttgggctggagtag |
| | | attttgaaagtcagctctgtgtgtgtgtgtgtgtgtgtgcgcgcgtgtgtttgtgtgtgtgtgagagcgtg |
| | | tgtttcttttaacgttttcagcctacagcatacagggttcatggtggcaagaagataacaagatttaaatt |
| | | atggccagtgactagtgctgcaagaagaacaactacctgcatttaatgggaaagcaaaatctcaggc |
| | | tttgagggaagttaacataggcttgattctgggtggaagcttggtgtgtagttatctggaggccaggct |
| | | ggagctctcagctcactatgggttcatctttattgtctcctttcatctcaacag |
| 13 | HBD intron 1 | gttggtatcaaggttataagagaggctcaaggaggcaaatggaaactgggcatgtgtagacagaga |
| | | agactcttgggtttctgataggcactgactctctgtcccttgggctgttttcctaccctcag |
| 14 | HBD intron 2 | gtgagtccaggagatgcttcacttttctctttttactttctaatcttacattttggttcttttacctacctgctctt |
| | | ctcccacatttttgtcattttactatattttatcatttaatgcttctaaaattttgttaattttttatttaaatattctg |
| | | cattttttccttcctcacaatcttgctattttaaattatttaatatcctgtctttctctcccaaccccctcccttca |
| | | tttttccttctctaacaacaactcaaattatgcataccagctctcacctgctaattctgcacttagaataatc |
| | | cttttgtctctccacatgggtatgggagaggctccaactcaaagatgagaggcatagaatactgtttta |
| | | gaggctataaatcattttacaataaggaataattggaattttataaattctgtagtaaatggaatggaaag |
| | | gaaagtgaatatttgattatgaaagactaggcagttacactggaggtggggcagaagtcgttgctagg |
| | | agacagcccatcatcacactgattaatcaattaatttgtatctattaatctgtttatagtaattaatttgtatat |
| | | gctatatacacatacaaaattaaaactaatttggaattaatttgtatatagtattatacagcatatatagcat |
| | | atatgtacatatatagactacatgctagttaagtacatagaggatgtgtgtgtatagatatatgttatatgt |
| | | atgcattcatatatgtacttatttatgctgatgggaataacctggggatcagttttgtctaagatttgggca |
| | | gaaaaaaatgggtgttggctcagtttctcagaagccagtctttatttctctgttaaccatatgcatgtatct |
| | | gcctacctcttctccgcag |
| 15 | Monkey- | gttggtatcaatgttataagagaggctcatggaggtaaatggaagctgggcatgtgtagacagagaa |
| | derived intron | gactctggaggttctgatagtcattgattctctctgtcccttgggctgttttcctaccctcag |
| | 1 | |
| 16 | Monkey- | gtgagtccaggagatgcttcacttttctctgtttactgtctaatcttacattttagtttttacctacctgctctt |
| | derived | cccccacatttttgtcattttactatattttatcatttaatgcttctaaaattttgttatttttttatttaaatattctg |
| | Intron 2 | cattttttccttcctcacaatcttgctattttaaattatttaatatcctgtcctctcctccccaaccccttccctt |
| | | cgttttcttctctaaccacaactcaaattatgcatgccagctctcacgtgctaattctgcacttagaataatt |
| | | ctttgtctctccacatgggtatgagagaggctccagctcaaagacgagaggcatagaatactgttttag |
| | | aggctataaattattttacaataaggaataattggaattttataaatttggtagtaaatgggatggaaagg |
| | | aaagtgaatatttgattatgaaagactagaaagttacactggaggtggggcagaagtcgttgctagga |
| | | gacagcccatcatcacactgattaatgaattaatttgtatctattaatctgtttagagtaattaatttgtatat |
| | | gctatatacacatacaaaattaaaactaatttggaattaatttgtatatagcattatacagcatatatagcat |
| | | atatgtacatatatagactatatgctagttaagtacacagaggatgtgtgtgtatagatatatgttatatgc |
| | | atgcattcatatatgtacttatttatgctgatgggaataacctggggatcagttttgtctaagatttgcgca |
| | | gaaaaaaatgggtgttggcccagtttctcagaagccaatctttatttctctgttaaccatatgcatatatct |
| | | gcctaccttttctctgcag |
| 17 | Split 5′ | Gtcctgtaagtattttgcatattctggagacgcaggaagagatccatctacatatcccaaagctgaatt |
| | Homology | atggtagacaaaactcttccacttttagtgcatcaacttcttatttgtgtaataagaaaattgggaaaac |
| | Arm | gatcttcaatatgcttaccaagctgtgattccaaatattacgtaaatacacttgcaaaggaggatgttttt |
| | | agtagcaatttgtactgatggtatggggccaagagatatatcttagagggagggctgagggtttgaa |
| | | gtccaactcctaagccagtgccagaagagccaaggacaggtacggctgtcatcacttagacctcac |
| | | cctgtggagccacaccctagggttggccaatctactcccaggagcagggagggcaggagccag |
| | | ggctgggcataaaagtcagggcagagccatctattgcttacatttgcttctgacacaactgtgttcact |
| | | agcaacctcaaacagacacc |
| 18 | Split 3′ | GCTCGCTTTCTTGCTGTCCAATTTCTATTAAAGGTTCCTTTGT |
| | Homology | TCCCTAAGTCCAACTACTAAACTGGGGGATATTATGAAGGG |
| | Arm | CCTTGAGCATCTGGATTCTGCCTAATAAAAAACATTTATTTT |
| | | CATTGCAATGATGTATTTAAATTATTTCTGAATATTTTACTA |
| | | AAAAGGGAATGTGGGAGGTCAGTGCATTTAAAACATAAAG |
| | | AAATGAAGAGCTAGTTCAAACCTTGGGAAAATACACTATAT |
| | | CTTAAACTCCATGAAAGAAGGTGAGGCTGCAAACAGCTAAT |
| | | GCACATTGGCAACAGCCCCTGATGCATATGCCTTATTCATC |
| | | CCTCAGAAAAGGATTCAAGTAGAGGCTTGATTTGGAGGTTA |
| | | AAGTTTTGCTATGCTGTATTTTACATTACTTATTGTTTTAGCT |
| | | GTCCTCATGAA |
| 19 | HBB | Gtcctgtaagtattttgcatattctggagacgcaggaagagatccatctacatatcccaaagctgaatt |
| | Homology 5′ | atggtagacaaaactcttccacttttagtgcatcaacttcttatttgtgtaataagaaaattgggaaaac |
| | Arm | gatcttcaatatgcttaccaagctgtgattccaaatattacgtaaatacacttgcaaaggaggatgttttt |
| | | agtagcaatttgtactgatggtatggggccaagagatatatcttagagggagggctgagggtttgaa |
| | | gtccaactcctaagccagtgccagaagagccaaggacaggtacggctgtcatcacttagacctcac |
| | | cctgtggagccacaccctagggttggccaatctactcccaggagcagggagggcaggagccag |
| | | ggctgggcataaaagtcagggcagagccatctattgcttacatttgcttctgacacaactgtgttcact |
| | | agcaacctcaaacagacacc |
| 20 | HBB | Ctgccctgtggggcaaggtgaacgtggatgaagttggtggtgaggccctgggcaggttggtatca |
| | Homology 3′ | aggttacaagacaggtttaaggagaccaatagaaactgggcatgtggagacagagaagactcttg |
| | Arm | ggtttctgataggcactgactctctctgcctattggtctattttcccacccttaggctgctggtggtctac |
| | | ccttggacccagaggttctttgagtcctttggggatctgtccactcctgatgctgttatgggcaaccct |
| | | aaggtgaaggctcatggcaagaaagtgctcggtgcctttagtgatggcctggctcacctggacaac |
| | | ctcaagggcacctttgccacactgagtgagctgcactgtgacaagctgcacgtggatcctgagaac |
| | | ttcagggtgagtctatgggacgct |
| 21 | HBB STOP 5′ | Cagggcaataatgatacaatgtatcatgcctctttgcaccattctaaagaataacagtgataatttctg |
| | Homology | ggttaaggcaatagcaatatctctgcatataaatatttctgcatataaattgtaactgatgtaagaggttt |
| | Arm | catattgctaatagcagctacaatccagctaccattctgcttttattttatggttgggataaggctggatt |
| | | attctgagtccaagctaggcccttttgctaatcatgttcatacctcttatcttcctcccacagCTCCT |
| | | GGGCAACGTGCTGGTCTGTGTGCTGGCCCATCACTTTGGCA |
| | | AAGAATTCACCCCACCAGTGCAGGCTGCCTATCAGAAAGTG |
| | | GTGGCTGGTGTGGCTAATGCCCTGGCTCATAAATACCAT |
| 22 | HBB STOP 3′ | GCTCGCTTTCTTGCTGTCCAATTTCTATTAAAGGTTCCTTTGT |
| | Homology | TCCCTAAGTCCAACTACTAAACTGGGGGATATTATGAAGGG |
| | Arm | CCTTGAGCATCTGGATTCTGCCTAATAAAAAACATTTATTTT |
| | | CATTGCAATGATGTATTTAAATTATTTCTGAATATTTTACTA |
| | | AAAAGGGAATGTGGGAGGTCAGTGCATTTAAAACATAAAG |
| | | AAATGAAGAGCTAGTTCAAACCTTGGGAAAATACACTATAT |
| | | CTTAAACTCCATGAAAGAAGGTGAGGCTGCAAACAGCTAAT |
| | | GCACATTGGCAACAGCCCCTGATGCATATGCCTTATTCATC |
| | | CCTCAGAAAAGGATTCAAGTAGAGGCTTGATTTGGAGGTTA |
| | | AAGTTTTGCTATGCTGTATTTTACA |
| 23 | HBA1 5′ | Gctccagccggttccagctattgctttgtttacctgtttaaccagtatttacctagcaagtcttccatcag |
| | Homology | atagcatttggagagctgggggtgtcacagtgaaccacgacctctaggccagtgggagagtcagt |
| | Arm | cacacaaactgtgagtccatgacttggggcttagccagcacccaccaccccacgcgccaccccac |
| | | aaccccgggtagaggagtctgaatctggagccgcccccagcccagccccgtgctttttgcgtcctg |
| | | gtgtttattccttcccggtgcctgtcactcaagcacactagtgactatcgccagagggaaagggagc |
| | | tgcaggaagcgaggctggagagcaggaggggctctgcgcagaaattcttttgagttcctatgggc |
| | | cagggcgtccgggtgcgcgcattcctctccgccccaggattgggcgaagcctcccggctcgcact |
| | | cgctcgcccgtgtgttccccgatcccgctggagtcgatgcgcgtccagcgcgtgccaggccggg |
| | | gcgggggtgcgggctgactttctccctcgctagggacgctccggcgcccgaaaggaaagggtgg |
| | | cgctgcgctccggggtgcacgagccgacagcgcccgaccccaacgggccggccccgccagcg |
| | | ccgctaccgccctgcccccgggcgagcgggatgggcgggagtggagtggcgggtggagggtg |
| | | gagacgtcctggcccccgccccgcgtgcacccccaggggaggccgagcccgccgcccggccc |
| | | cgcgcaggccccgcccgggactcccctgcggtccaggccgcgccccgggctccgcgccagcc |
| | | aatgagcgccgcccggccgggcgtgcccccgcgccccaagcataaaccctggcgcgctcgcgg |
| | | cccggcactcttctggtccccacagactcagagagaacccacc |
| 24 | HBA1 3′ | tggccatgcttcttgccccttgggcctccccccagcccctcctccccttcctgcacccgtacccccgt |
| | Homology | ggtctttgaataaagtctgagtgggcggcagcctgtgtgtgcctgagttttttccctcagcaaacgtgc |
| | Arm | caggcatgggcgtggacagcagctgggacacacatggctagaacctctctgcagctggatagggt |
| | | aggaaaaggcaggggcgggaggaggggatggaggagggaaagtggagccaccgcgaagtcc |
| | | agctggaaaaacgctggaccctagagtgctttgaggatgcatttgctctttcccgagttttattcccag |
| | | acttttcagattcaatgcaggtttgctgaaataatgaatttatccatctttacgtttctgggcactctgtgc |
| | | caagaactggctggctttctgcctgggacgtcactggtttcccagaggtcctcccacatatgggtggt |
| | | gggtaggtcagagaagtcccactccagcatggctgcattgatcccccatcgttcccactagtctccg |
| | | taaaacctcccagatacaggcacagtctagatgaaatcaggggtgcggggtgcaactgcaggccc |
| | | caggcaattcaataggggctctactttcacccccaggtcaccccagaatgctcacacaccagacac |
| | | tgacgccctggggctgtcaagatcaggcgtttgtctctgggcccagctcagggcccagctcagcac |
| | | ccactcagctcccctgaggctggggagcctgtcccattgcgactggagaggagagcggggccac |
| | | agaggcctggctagaaggtcccttctccctggtgtgtgttttctctctgctgagcaggcttgcagtgcc |
| | | tggggtatca |
| 25 | HBA1 target | GGCAAGAAGCATGGCCACCG |
| | sequence for | |
| | sgRNAagainst | |
| | HBA1 | |
| 26 | HBB target | AGCGAGCTTAGTGATACTTG |
| | sequence for | |
| | sgRNAagainst | |
| | HBB-STOP | |
| 27 | HBB target | CTTGCCCCACAGGGCAGTAA |
| | sequence for | |
| | sgRNAagainst | |
| | HBB exon 1 | |
| 28 | HBA1 Intron 1 | gtgaggctccctcccctgctccgacccgggctcctcgcccgcccggacccacaggccaccctca |
| | | accgtcctggccccggacccaaaccccacccctcactctgcttctccccgcag |
| 29 | HBA1 Intron 2 | gtgagcggcgggccgggagcgatctgggtcgaggggcgagatggcgccttcctcgcagggca |
| | | gaggatcacgcgggttgcgggaggtgtagcgcaggcggcggctgcgggcctgggccctcggc |
| | | cccactgaccctcttctctgcacag |
| 30 | HBB (CDS | ATGGTGCACCTGACTCCTGAGGAGAAGTCTGCCGTGACCGC |
| | diverged with | TCTCTGGGGAAAAGTCAATGTCGACGAGGTGGGAGGCGAA |
| | HBA1 Introns) | GCTCTCGGAAGgtgaggctccctcccctgctccgacccgggctcctcgcccgcccgga |
| | | cccacaggccaccctcaaccgtcctggccccggacccaaaccccacccctcactctgcttctcccc |
| | | gcagACTCCTCGTCGTGTATCCCTGGACACAAAGATTTTTCGA |
| | | AAGCTTCGGCGACCTCAGCACACCCGACGCCGTGATGGGGA |
| | | ATCCCAAAGTCAAAGCCCACGGGAAAAAGGTCCTGGGCGC |
| | | TTTCTCCGACGGACTCGCCCATCTCGATAATCTGAAAGGAA |
| | | CTTTCGCTACCCTCTCCGAACTCCATTGCGATAAACTCCATG |
| | | TCGACCCCGAAAATTTTAGAgtgagcggcgggccgggagcgatctgggtcga |
| | | ggggcgagatggcgccttcctcgcagggcagaggatcacgcgggttgcgggaggtgtagcgca |
| | | ggcggcggctgcgggcctgggccctcggccccactgaccctcttctctgcacagCTGCTCG |
| | | GGAATGTCCTCGTGTGCGTCCTCGCTCACCATTTCGGGAAG |
| | | GAGTTTACACCCCCCGTCCAAGCCGCTTACCAAAAGGTCGT |
| | | CGCCGGAGTCGCCAACGCTCTCGCTCATAAATACCATTAA |
| 31 | EGFP | ATGGTGAGCAAGGGCGAGGAGCTGTTCACCGGGGTGGTGC |
| | | CCATCCTGGTCGAGCTGGACGGCGACGTAAACGGCCACAA |
| | | GTTCAGCGTGTCCGGCGAGGGCGAGGGCGATGCCACCTACG |
| | | GCAAGCTGACCCTGAAGTTCATCTGCACCACCGGCAAGCTG |
| | | CCCGTGCCCTGGCCCACCCTCGTGACCACCCTGACCTACGG |
| | | CGTGCAGTGCTTCAGCCGCTACCCCGACCACATGAAGCAGC |
| | | ACGACTTCTTCAAGTCCGCCATGCCCGAAGGCTACGTCCAG |
| | | GAGCGCACCATCTTCTTCAAGGACGACGGCAACTACAAGAC |
| | | CCGCGCCGAGGTGAAGTTCGAGGGCGACACCCTGGTGAAC |
| | | CGCATCGAGCTGAAGGGCATCGACTTCAAGGAGGACGGCA |
| | | ACATCCTGGGGCACAAGCTGGAGTACAACTACAACAGCCA |
| | | CAACGTCTATATCATGGCCGACAAGCAGAAGAACGGCATC |
| | | AAGGTGAACTTCAAGATCCGCCACAACATCGAGGACGGCA |
| | | GCGTGCAGCTCGCCGACCACTACCAGCAGAACACCCCCATC |
| | | GGCGACGGCCCCGTGCTGCTGCCCGACAACCACTACCTGAG |
| | | CACCCAGTCCGCCCTGAGCAAAGACCCCAACGAGAAGCGC |
| | | GATCACATGGTCCTGCTGGAGTTCGTGACCGCCGCCGGGAT |
| | | CACTCTCGGCATGGACGAGCTGTACAAGTAA |
| 32 | bGH | CTGTGCCTTCTAGTTGCCAGCCATCTGTTGTTTGCCCCTCCC |
| | polyadenyl- | CCGTGCCTTCCTTGACCCTGGAAGGTGCCACTCCCACTGTCC |
| | ation | TTTCCTAATAAAATGAGGAAATTGCATCGCATTGTCTGAGT |
| | sequence | AGGTGTCATTCTATTCTGGGGGGTGGGGTGGGGCAGGACAG |
| | | CAAGGGGGAGGATTGGGAAGACAATAGCAGGCATGCTGGG |
| | | GA |
| 33 | WPRE | AATCAACCTCTGGATTACAAAATTTGTGAAAGATTGACTGG |
| | sequence with | TATTCTTAACTATGTTGCTCCTTTTACGCTATGTGGATACGC |
| | short poly A | TGCTTTAATGCCTTTGTATCATGCTATTGCTTCCCGTATGGC |
| | sequence | TTTCATTTTCTCCTCCTTGTATAAATCCTGGTTGCTGTCTCTT |
| | | TATGAGGAGTTGTGGCCCGTTGTCAGGCAACGTGGCGTGGT |
| | | GTGCACTGTGTTTGCTGACGCAACCCCCACTGGTTGGGGCA |
| | | TTGCCACCACCTGTCAGCTCCTTTCCGGGACTTTCGCTTTCC |
| | | CCCTCCCTATTGCCACGGCGGAACTCATCGCCGCCTGCCTTG |
| | | CCCGCTGCTGGACAGGGGCTCGGCTGTTGGGCACTGACAAT |
| | | TCCGTGGTGTTGTCGGGGAAGCTGACGTCCTTTCCATGGCT |
| | | GCTCGCCTGTGTTGCCACCTGGATTCTGCGCGGGACGTCCTT |
| | | CTGCTACGTCCCTTCGGCCCTCAATCCAGCGGACCTTCCTTC |
| | | CCGCGGCCTGCTGCCGGCTCTGCGGCCTCTTCCGCGTCTTCG |
| | | CCTTCGCCCTCAGACGAGTCGGATCTCCCTTTGGGCCGCCTC |
| | | CCCGCaataaaagatctttattttcattagatctgtgtgttggttttttgtgtg |
| 34 | HBB Exon 1 | ATGGTGCATCTGACTCCTGAGGAGAAGTCTGCCGTTACTGC |
| | (WT) | CCTGTGGGGCAAGGTGAACGTGGATGAAGTTGGTGGTGAG |
| | | GCCCTGGGCAG |
| 35 | HBB Diverged | ATGGTcCAcCTcACcCCcGAaGAaAAaTCcGCaGTcACcGCTCTC |
| | Exon 1 | TGGGGAAAAGTCAATGTCGACGAGGTGGGAGGCGAAGCTC |
| | | TCGGAAG |
| 36 | HBB Diverged | ACTCCTCGTCGTGTATCCCTGGACACAAAGATTTTTCGAAA |
| | Exon 2 | GCTTCGGCGACCTCAGCACACCCGACGCCGTGATGGGGAAT |
| | | CCCAAAGTCAAAGCCCACGGGAAAAAGGTCCTGGGCGCTTT |
| | | CTCCGACGGACTCGCCCATCTCGATAATCTGAAAGGAACTT |
| | | TCGCTACCCTCTCCGAACTCCATTGCGATAAACTCCATGTCG |
| | | ACCCCGAAAATTTTAGA |
| 37 | HBB Diverged | CTGCTCGGGAATGTCCTCGTGTGCGTCCTCGCTCACCATTTC |
| | Exon 3 | GGGAAGGAGTTTACACCCCCCGTCCAAGCCGCTTACCAAAA |
| | | GGTCGTCGCCGGAGTCGCCAACGCTCTCGCTCATAAATACC |
| | | AT |
| 38 | β-SCD-SNP | GTGGAAAAATCCGCAGTCA |
| | donor | |
| | sequence (E6V) | |
| 39 | β-SCD-SNP 5′ | AATTTAGTACAAGGGGAAAAAGTACAGGGGGATGGGAGAA |
| | Homology Arm | AGGCGATCACGTTGGGAAGCTATAGAGAAAGAAGAGTAAA |
| | | TTTTAGTAAAGGAGGTTTAAACAAACAAAATATAAAGAGA |
| | | AATAGGAACTTGAATCAAGGAAATGATTTTAAAACGCAGTA |
| | | TTCTTAGTGGACTAGAGGAAAAAAATAATCTGAGCCAAGTA |
| | | GAAGACCTTTTCCCCTCCTACCCCTACTTTCTAAGTCACAGA |
| | | GGCTTTTTGTTCCCCCAGACACTCTTGCAGATTAGTCCAGGC |
| | | AGAAACAGTTAGATGTCCCCAGTTAACCTCCTATTTGACAC |
| | | CACTGATTACCCCATTGATAGTCACACTTTGGGTTGTAAGTG |
| | | ACTTTTTATTTATTTGTATTTTTGACTGCATTAAGAGGTCTCT |
| | | AGTTTTTTACCTCTTGTTTCCCAAAACCTAATAAGTAACTAA |
| | | TGCACAGAGCACATTGATTTGTATTTATTCTATTTTTAGACA |
| | | TAATTTATTAGCATGCATGAGCAAATTAAGAAAAACAACAA |
| | | CAAATGAATGCATATATATGTATATGTATGTGTGTACATAT |
| | | ACACATATATATATATATTTTTTTTCTTTTCTTACCAGAAGG |
| | | TTTTAATCCAAATAAGGAGAAGATATGCTTAGAACTGAGGT |
| | | AGAGTTTTCATCCATTCTGTCCTGTAAGTATTTTGCATATTC |
| | | TGGAGACGCAGGAAGAGATCCATCTACATATCCCAAAGCTG |
| | | AATTATGGTAGACAAAACTCTTCCACTTTTAGTGCATCAATT |
| | | TCTTATTTGTGTAATAAGAAAATTGGGAAAACGATCTTCAA |
| | | TATGCTTACCAAGCTGTGATTCCAAATATTACGTAAATACA |
| | | CTTGCAAAGGAGGATGTTTTTAGTAGCAATTTGTACTGATG |
| | | GTATGGGGCCAAGAGATATATCTTAGAGGGAGGGCTGAGG |
| | | GTTTGAAGTCCAACTCCTAAGCCAGTGCCAGAAGAGCCAAG |
| | | GACAGGTACGGCTGTCATCACTTAGACCTCACCCTGTGGAG |
| | | CCACACCCTAGGGTTGGCCAATCTACTCCCAGGAGCAGGGA |
| | | GGGCAGGAGCCAGGGCTGGGCATAAAAGTCAGGGCAGAGC |
| | | CATCTATTGCTTACATTTGCTTCTGACACAACTGTGTTCACT |
| | | AGCAACCTCAAACAGACACCATGGTGCACCTGACTCCT |
| 40 | β-SCD-SNP 3′ | CTGCCCTGTGGGGCAAGGTGAACGTGGATGAAGTTGGTGGT |
| | Homology Arm | GAGGCCCTGGGCAGgttggtatcaaggttacaagacaggtttaaggagaccaatagaa |
| | | actgggcatgtggagacagagaagactcttgggtttctgataggcactgactctctctgcctattggt |
| | | ctattttcccacccttagGCTGCTGGTGGTCTACCCTTGGACCCAGAGG |
| | | ttctttgagtcctttggggatctgtccactcctgatgctgtt |
| | | ATGGGCAACCCTAAGGTGAAGGCTCATGGCAAGAAAGTGC |
| | | TCGGTGCCTTTAGTGATGGCCTGGCTCACCTGGACAACCTC |
| | | AAGGGCACCTTTGCCACACTGAGTGAGCTGCACTGTGACAA |
| | | GCTGCACGTGGATCCTGAGAACTTCAGGgtgagtctatgggacccttgat |
| | | gttttctttccccttcttttctatggttaagttcatgtcataggaaggggataagtaacagggtacagttta |
| | | gaatgggaaacagacgaatgattgcatcagtgtggaagtctcaggatcgttttagtttcttttatttgctgtt |
| | | cataacaattgttttcttttgtttaattcttgctttctttttttttcttctccgcaatttttactattatacttaat |
| | | gccttaacattgtgtataacaaaaggaaatatctctgagatacattaagtaacttaaaaaaaaactttac |
| | | acagtctgcctagtacattactatttggaatatatgtgtgcttatttgcatattcataatctccctactttattt |
| | | tcttttatttttaattgatacataatcattatacatatttatgggttaaagtgtaatgttttaatatgtgtacaca |
| | | tattgaccaaatcagggtaattttgcatttgtaattttaaaaaatgctttcttcttttaatatacttttttgtttat |
| | | cttatttctaatactttccctaatctctttctttcagggcaataatgatacaatgtatcatgcctctttgcacc |
| | | attctaaagaataacagtgataatttctgggttaaggcaatagcaatatctctgcatataaatatttctgc |
| | | atataaattgtaactgatgtaagaggtttcatattgctaatagcagctacaatccagctaccattctgctt |
| | | ttattttatggttgggataaggctgg |
| 41 | β-SCD-HB^{div}- | ATGGTCCACCTCACCCCCGTGGAAAAATCCGCAGTCACCGC |
| | NoIntrons-bGH | TCTCTGGGGAAAAGTCAATGTCGACGAGGTGGGAGGCGAA |
| | donor | GCTCTCGGAAGACTCCTCGTCGTGTATCCCTGGACACAAAG |
| | sequence | ATTTTTCGAAAGCTTCGGCGACCTCAGCACACCCGACGCCG |
| | | TGATGGGGAATCCCAAAGTCAAAGCCCACGGGAAAAAGGT |
| | | CCTGGGCGCTTTCTCCGACGGACTCGCCCATCTCGATAATCT |
| | | GAAAGGAACTTTCGCTACCCTCTCCGAACTCCATTGCGATA |
| | | AACTCCATGTCGACCCCGAAAATTTTAGACTGCTCGGGAAT |
| | | GTCCTCGTGTGCGTCCTCGCTCACCATTTCGGGAAGGAGTTT |
| | | ACACCCCCCGTCCAAGCCGCTTACCAAAAGGTCGTCGCCGG |
| | | AGTCGCCAACGCTCTCGCTCATAAATACCATTAA |
| 42 | β-SCD- | ATGGTCCACCTCACCCCCGTGGAAAAATCCGCAGTCACCGC |
| | HBB^{div}HBG2^{intr}- | TCTCTGGGGAAAAGTCAATGTCGACGAGGTGGGAGGCGAA |
| | bGH donor | GCTCTCGGAAGgtaggctctggtgaccaggacaagggagggaaggaaggaccctgtg |
| | sequence | cctggcaaaagtccaggtcgcttctcaggatttgtggcaccttctgactgtcaaactgttcttgtcaatc |
| | | tcacagACTCCTCGTCGTGTATCCCTGGACACAAAGATTTTTCG |
| | | AAAGCTTCGGCGACCTCAGCACACCCGACGCCGTGATGGGG |
| | | AATCCCAAAGTCAAAGCCCACGGGAAAAAGGTCCTGGGCG |
| | | CTTTCTCCGACGGACTCGCCCATCTCGATAATCTGAAAGGA |
| | | ACTTTCGCTACCCTCTCCGAACTCCATTGCGATAAACTCCAT |
| | | GTCGACCCCGAAAATTTTAGAgtgagtccaggagatgtttcagcactgttgcctt |
| | | tagtctcgaggcaacttagacaactgagtattgatctgagcacagcagggtgtgagctgtttgaagat |
| | | actggggttgggagtgaagaaactgcagaggactaactgggctgagacccagtggcaatgttttag |
| | | ggcctaaggagtgcctctgaaaatctagatggacaactttgactttgagaaaagagaggtggaaat |
| | | gaggaaaatgacttttctttattagatttcggtagaaagaactttcacctttcccctatttttgttattcgtttt |
| | | aaaacatctatctggaggcaggacaagtatggtcattaaaaagatgcaggcagaaggcatatattg |
| | | gctcagtcaaagtggggaactttggtggccaaacatacattgctaaggctattcctatatcagctgga |
| | | cacatataaaatgctgctaatgcttcattacaaacttatatcctttaattccagatgggggcaaagtatgt |
| | | ccaggggtgaggaacaattgaaacatttgggctggagtagattttgaaagtcagctctgtgtgtgtgt |
| | | gtgtgtgtgtgcgcgcgtgtgtttgtgtgtgtgtgagagcgtgtgtttcttttaacgttttcagcctacag |
| | | catacagggttcatggtggcaagaagataacaagatttaaattatggccagtgactagtgctgcaag |
| | | aagaacaactacctgcatttaatgggaaagcaaaatctcaggctttgagggaagttaacataggctt |
| | | gattctgggtggaagcttggtgtgtagttatctggaggccaggctggagctctcagctcactatgggt |
| | | tcatctttattgtctcctttcatctcaacagCTGCTCGGGAATGTCCTCGTGTGC |
| | | GTCCTCGCTCACCATTTCGGGAAGGAGTTTACACCCCCCGT |
| | | CCAAGCCGCTTACCAAAAGGTCGTCGCCGGAGTCGCCAACG |
| | | CTCTCGCTCATAAATACCATTAA |
| 43 | β-SCD- | ATGGTCCACCTCACCCCCGTGGAAAAATCCGCAGTCACCGC |
| | HBB^{div}HBD^{intr}- | TCTCTGGGGAAAAGTCAATGTCGACGAGGTGGGAGGCGAA |
| | bGH | GCTCTCGGAAGgttggtatcaaggttataagagaggctcaaggaggcaaatggaaactg |
| | | ggcatgtgtagacagagaagactcttgggtttctgataggcactgactctctgtcccttgggctgtttt |
| | | cctaccctcagACTCCTCGTCGTGTATCCCTGGACACAAAGATTTT |
| | | TCGAAAGCTTCGGCGACCTCAGCACACCCGACGCCGTGATG |
| | | GGGAATCCCAAAGTCAAAGCCCACGGGAAAAAGGTCCTGG |
| | | GCGCTTTCTCCGACGGACTCGCCCATCTCGATAATCTGAAA |
| | | GGAACTTTCGCTACCCTCTCCGAACTCCATTGCGATAAACTC |
| | | CATGTCGACCCCGAAAATTTTAGAgtgagtccaggagatgcttcacttttctct |
| | | ttttactttctaatcttacattttggttcttttacctacctgctcttctcccacatttttgtcattttactatatttta |
| | | tcatttaatgcttctaaaattttgttaattttttatttaaatattctgcattttttccttcctcacaatcttgctattt |
| | | taaattatttaatatcctgtctttctctcccaaccccctcccttcatttttccttctctaacaacaactcaaat |
| | | tatgcataccagctctcacctgctaattctgcacttagaataatccttttgtctctccacatgggtatggg |
| | | agaggctccaactcaaagatgagaggcatagaatactgttttagaggctataaatcattttacaataa |
| | | ggaataattggaattttataaattctgtagtaaatggaatggaaaggaaagtgaatatttgattatgaaa |
| | | gactaggcagttacactggaggtggggcagaagtcgttgctaggagacagcccatcatcacactg |
| | | attaatcaattaatttgtatctattaatctgtttatagtaattaatttgtatatgctatatacacatacaaaatt |
| | | aaaactaatttggaattaatttgtatatagtattatacagcatatatagcatatatgtacatatatagacta |
| | | catgctagttaagtacatagaggatgtgtgtgtatagatatatgttatatgtatgcattcatatatgtactt |
| | | atttatgctgatgggaataacctggggatcagttttgtctaagatttgggcagaaaaaaatgggtgttg |
| | | gctcagtttctcagaagccagtctttatttctctgttaaccatatgcatgtatctgcctacctcttctccgc |
| | | agCTGCTCGGGAATGTCCTCGTGTGCGTCCTCGCTCACCATT |
| | | TCGGGAAGGAGTTTACACCCCCCGTCCAAGCCGCTTACCAA |
| | | AAGGTCGTCGCCGGAGTCGCCAACGCTCTCGCTCATAAATA |
| | | CCATTAA |
| 44 | β-SCD- | ATGGTCCACCTCACCCCCGTGGAAAAATCCGCAGTCACCGC |
| | HBB^{div}monkey^{intr}- | TCTCTGGGGAAAAGTCAATGTCGACGAGGTGGGAGGCGAA |
| | bGH | GCTCTCGGAAGgttggtatcaatgttataagagaggctcatggaggtaaatggaagctgg |
| | | gcatgtgtagacagagaagactctggaggttctgatagtcattgattctctctgtcccttgggctgtttt |
| | | cctaccctcagACTCCTCGTCGTGTATCCCTGGACACAAAGATTTT |
| | | TCGAAAGCTTCGGCGACCTCAGCACACCCGACGCCGTGATG |
| | | GGGAATCCCAAAGTCAAAGCCCACGGGAAAAAGGTCCTGG |
| | | GCGCTTTCTCCGACGGACTCGCCCATCTCGATAATCTGAAA |
| | | GGAACTTTCGCTACCCTCTCCGAACTCCATTGCGATAAACTC |
| | | CATGTCGACCCCGAAAATTTTAGAgtgagtccaggagatgcttcacttttctct |
| | | gtttactgtctaatcttacattttagtttttacctacctgctcttcccccacatttttgtcattttactatattttat |
| | | catttaatgcttctaaaattttgttatttttttatttaaatattctgcattttttccttcctcacaatcttgctatttt |
| | | aaattatttaatatcctgtcctctcctccccaaccccttcccttcgttttcttctctaaccacaactcaaatt |
| | | atgcatgccagctctcacgtgctaattctgcacttagaataattctttgtctctccacatgggtatgaga |
| | | gaggctccagctcaaagacgagaggcatagaatactgttttagaggctataaattattttacaataag |
| | | gaataattggaattttataaatttggtagtaaatgggatggaaaggaaagtgaatatttgattatgaaag |
| | | actagaaagttacactggaggtggggcagaagtcgttgctaggagacagcccatcatcacactgat |
| | | taatgaattaatttgtatctattaatctgtttagagtaattaatttgtatatgctatatacacatacaaaatta |
| | | aaactaatttggaattaatttgtatatagcattatacagcatatatagcatatatgtacatatatagactat |
| | | atgctagttaagtacacagaggatgtgtgtgtatagatatatgttatatgcatgcattcatatatgtactta |
| | | tttatgctgatgggaataacctggggatcagttttgtctaagatttgcgcagaaaaaaatgggtgttgg |
| | | cccagtttctcagaagccaatctttatttctctgttaaccatatgcatatatctgcctaccttttctctgcag |
| | | CTGCTCGGGAATGTCCTCGTGTGCGTCCTCGCTCACCATTTC |
| | | GGGAAGGAGTTTACACCCCCCGTCCAAGCCGCTTACCAAAA |
| | | GGTCGTCGCCGGAGTCGCCAACGCTCTCGCTCATAAATACC |
| | | ATTAA |
| 45 | HBE1 poly A | GTTCTCTTCCAGTTTGCAGGTGTTCCTGTGACCCTGACACCC |
| | | TCCTTCTGCACATGGGGACTGGGCTTGGCCTTGAGAGAAAG |
| | | CCTTCTGTTTAATAAAGTACATTTTCTTCAGTAATCAAAAA |
| 46 | HBG2 poly A | GCTCACTGCCCATGATGCAGAGCTTTCAAGGATAGGCTTTA |
| | | TTCTGCAAGCAATCAAATAATAAATCTATTCTGCTAAGAGA |
| | | TCACACA |
| 47 | HBG1 poly A | GCTCACTGCCCATGATTCAGAGCTTTCAAGGATAGGCTTTA |
| | | TTCTGCAAGCAATACAAATAATAAATCTATTCTGCTGAGAG |
| | | ATCACACA |
| 48 | HBD poly A | GATCCTGGACTGTTTCCTGATAACCATAAGAAGACCCTATT |
| | | TCCCTAGATTCTATTTTCTGAACTTGGGAACACAATGCCTAC |
| | | TTCAAGGGTATGGCTTCTGCCTAATAAAGAATGTTCAGCTC |
| | | AA |
| 49 | HBZ poly A | GCGCCGCCTCCGGGACCCCCAGGACAGGCTGCGGCCCCTCC |
| | | CCCGTCCTGGAGGTTCCCCAGCCCCACTTACCGCGTAATGC |
| | | GCCAATAAACCAATGAACGAA |
| 50 | HBA2 poly A | GCTGGAGCCTCGGTAGCCGTTCCTCCTGCCCGCTGGGCCTC |
| | | CCAACGGGCCCTCCTCCCCTCCTTGCACCGGCCCTTCCTGGT |
| | | CTTTGAATAAAGTCTGAGTGGGCAGCA |
| 51 | HBA1 poly A | GCTGGAGCCTCGGTGGCCATGCTTCTTGCCCCTTGGGCCTCC |
| | | CCCCAGCCCCTCCTCCCCTTCCTGCACCCGTACCCCCGTGGT |
| | | CTTTGAATAAAGTCTGAGTGGGCGGCA |
| 52 | hGH poly A | gggtggcatccctgtgacccctccccagtgcctctcctggccctggaagttgccactccagtgccc |
| | | accagccttgtcctaataaaattaagttgcatcattttgtctgactaggtgtccttctataatattatgggg |
| | | tggaggggggtggtatggagcaaggggcaagttgggaagacaacctgtagggcctgcggggtct |
| | | attgggaaccaagctggagtgcagtggcacaatcttggctcactgcaatctccgcctcctgggttca |
| | | agcgattctcctgcctcagcctcccgagttgttgggattccaggcatgcatgaccaggctcagctaat |
| | | ttttgtttttttggtagagacggggtttcaccatattggccaggctggtctccaactcctaatctcaggtg |
| | | atctacccaccttggcctcccaaattgctgggattacaggcgtgaaccactgctcccttccctgtcctt |
| 53 | RbGlob poly A | aataaaggaaatttattttcattgcaatagtgtgttggaattttttgtgtctctca |
| 54 | SynthRbGlob | AATAAAAGATCTTTATTTTCATTAGATCTGTGTGTTGGTTTT |
| | poly A | TTGTGTG |
| 55 | SV40 poly A | aacttgtttattgcagcttataatggttacaaataaagcaatagcatcacaaatttcacaaataaagcatt |
| | | tttttcactgcattctagttgtggtttgtccaaactcatcaatgtatctta |
| 56 | HBB (CDS | ATGGTcCAcCTcACcCCcGAaGAaAAaTCcGCaGTcACcGCTCTC |
| | diverged with | TGGGGAAAAGTCAATGTCGACGAGGTGGGAGGCGAAGCTC |
| | WTHBG2 | TCGGAAGgtaggctctggtgaccaggacaagggagggaaggaaggaccctgtgcctggc |
| | introns) | aaaagtccaggtcgcttctcaggatttgtggcaccttctgactgtcaaactgttcttgtcaatctcacag |
| | | ACTCCTCGTCGTGTATCCCTGGACACAAAGATTTTTCGAAA |
| | | GCTTCGGCGACCTCAGCACACCCGACGCCGTGATGGGGAAT |
| | | CCCAAAGTCAAAGCCCACGGGAAAAAGGTCCTGGGCGCTTT |
| | | CTCCGACGGACTCGCCCATCTCGATAATCTGAAAGGAACTT |
| | | TCGCTACCCTCTCCGAACTCCATTGCGATAAACTCCATGTCG |
| | | ACCCCGAAAATTTTAGAgtgagtccaggagatgtttcagcactgttgcctttagtctc |
| | | gaggcaacttagacaactgagtattgatctgagcacagcagggtgtgagctgtttgaagatactggg |
| | | gttgggagtgaagaaactgcagaggactaactgggctgagacccagtggcaatgttttagggccta |
| | | aggagtgcctctgaaaatctagatggacaactttgactttgagaaaagagaggtggaaatgaggaa |
| | | aatgacttttctttattagatttcggtagaaagaactttcacctttcccctatttttgttattcgttttaaaaca |
| | | tctatctggaggcaggacaagtatggtcattaaaaagatgcaggcagaaggcatatattggctcagt |
| | | caaagtggggaactttggtggccaaacatacattgctaaggctattcctatatcagctggacacatat |
| | | aaaatgctgctaatgcttcattacaaacttatatcctttaattccagatgggggcaaagtatgtccagg |
| | | ggtgaggaacaattgaaacatttgggctggagtagattttgaaagtcagctctgtgtgtgtgtgtgtgt |
| | | gtgtgcgcgcgtgtgtttgtgtgtgtgtgagagcgtgtgtttcttttaacgttttcagcctacagcataca |
| | | gggttcatggtggcaagaagataacaagatttaaattatggccagtgactagtgctgcaagaagaac |
| | | aactacctgcatttaatgggaaagcaaaatctcaggctttgagggaagttaacataggcttgattctg |
| | | ggtggaagcttggtgtgtagttatctggaggccaggctggagctctcagctcactatgggttcatcttt |
| | | attgtctcctttcatctcaacagCTGCTCGGGAATGTCCTCGTGTGCGTCCT |
| | | CGCTCACCATTTCGGGAAGGAGTTTACACCCCCCGTCCAAG |
| | | CCGCTTACCAAAAGGTCGTCGCCGGAGTCGCCAACGCTCTC |
| | | GCTCATAAATACCAT |
| 57 | HBB (CDS | ATGGTGCACCTGACTCCTGAGGAGAAGTCTGCCGTGACCGC |
| | diverged with | TCTCTGGGGAAAAGTCAATGTCGACGAGGTGGGAGGCGAA |
| | no introns | GCTCTCGGAAGACTCCTCGTCGTGTATCCCTGGACACAAAG |
| | (Aint1Aint2)) | ATTTTTCGAAAGCTTCGGCGACCTCAGCACACCCGACGCCG |
| | | TGATGGGGAATCCCAAAGTCAAAGCCCACGGGAAAAAGGT |
| | | CCTGGGCGCTTTCTCCGACGGACTCGCCCATCTCGATAATCT |
| | | GAAAGGAACTTTCGCTACCCTCTCCGAACTCCATTGCGATA |
| | | AACTCCATGTCGACCCCGAAAATTTTAGACTGCTCGGGAAT |
| | | GTCCTCGTGTGCGTCCTCGCTCACCATTTCGGGAAGGAGTTT |
| | | ACACCCCCCGTCCAAGCCGCTTACCAAAAGGTCGTCGCCGG |
| | | AGTCGCCAACGCTCTCGCTCATAAATACCAT |
| 58 | HBB (CDS | ATGGTCCACCTCACCCCCGAAGAAAAATCCGCAGTCACCGC |
| | diverged with | TCTCTGGGGAAAAGTCAATGTCGACGAGGTGGGAGGCGAA |
| | HBG2 intron 2 | GCTCTCGGAAGACTCCTCGTCGTGTATCCCTGGACACAAAG |
| | only (Aint1) | ATTTTTCGAAAGCTTCGGCGACCTCAGCACACCCGACGCCG |
| | | TGATGGGGAATCCCAAAGTCAAAGCCCACGGGAAAAAGGT |
| | | CCTGGGCGCTTTCTCCGACGGACTCGCCCATCTCGATAATCT |
| | | GAAAGGAACTTTCGCTACCCTCTCCGAACTCCATTGCGATA |
| | | AACTCCATGTCGACCCCGAAAATTTTAGAgtgagtccaggagatgtttc |
| | | agcactgttgcctttagtctcgaggcaacttagacaactgagtattgatctgagcacagcagggtgtg |
| | | agctgtttgaagatactggggttgggagtgaagaaactgcagaggactaactgggctgagaccca |
| | | gtggcaatgttttagggcctaaggagtgcctctgaaaatctagatggacaactttgactttgagaaaa |
| | | gagaggtggaaatgaggaaaatgacttttctttattagatttcggtagaaagaactttcacctttcccct |
| | | atttttgttattcgttttaaaacatctatctggaggcaggacaagtatggtcattaaaaagatgcaggca |
| | | gaaggcatatattggctcagtcaaagtggggaactttggtggccaaacatacattgctaaggctattc |
| | | ctatatcagctggacacatataaaatgctgctaatgcttcattacaaacttatatcctttaattccagatg |
| | | ggggcaaagtatgtccaggggtgaggaacaattgaaacatttgggctggagtagattttgaaagtc |
| | | agctctgtgtgtgtgcgcgcgtgtgtttgtgtgtgtgtgagagcgtgtgtttcttttaacgttttcagccta |
| | | cagcatacagggttcatggtggcaagaagataacaagatttaaattatggccagtgactagtgctgc |
| | | aagaagaacaactacctgcatttaatgggaaagcaaaatctcaggctttgagggaagttaacatagg |
| | | cttgattctgggtggaagcttggtgtgtagttatctggaggccaggctggagctctcagctcactatg |
| | | ggttcatctttattgtctcctttcatctcaacagCTGCTCGGGAATGTCCTCGTGTG |
| | | CGTCCTCGCTCACCATTTCGGGAAGGAGTTTACACCCCCCG |
| | | TCCAAGCCGCTTACCAAAAGGTCGTCGCCGGAGTCGCCAAC |
| | | GCTCTCGCTCATAAATACCAT |
| 59 | HBB (CDS | ATGGTCCACCTCACCCCCGAAGAAAAATCCGCAGTCACCGC |
| | diverged with | TCTCTGGGGAAAAGTCAATGTCGACGAGGTGGGAGGCGAA |
| | HBG2 intron 1 | GCTCTCGGAAGgtaggctctggtgaccaggacaagggagggaaggaaggaccctgtg |
| | only (Aint2) | cctggcaaaagtccaggtcgcttctcaggatttgtggcaccttctgactgtcaaactgttcttgtcaatc |
| | | tcacagACTCCTCGTCGTGTATCCCTGGACACAAAGATTTTTCG |
| | | AAAGCTTCGGCGACCTCAGCACACCCGACGCCGTGATGGGG |
| | | AATCCCAAAGTCAAAGCCCACGGGAAAAAGGTCCTGGGCG |
| | | CTTTCTCCGACGGACTCGCCCATCTCGATAATCTGAAAGGA |
| | | ACTTTCGCTACCCTCTCCGAACTCCATTGCGATAAACTCCAT |
| | | GTCGACCCCGAAAATTTTAGACTGCTCGGGAATGTCCTCGT |
| | | GTGCGTCCTCGCTCACCATTTCGGGAAGGAGTTTACACCCC |
| | | CCGTCCAAGCCGCTTACCAAAAGGTCGTCGCCGGAGTCGCC |
| | | AACGCTCTCGCTCATAAATACCAT |
| 60 | HBB (CDS | ATGGTCCACCTCACCCCCGAAGAAAAATCCGCAGTCACCGC |
| | diverged with | TCTCTGGGGAAAAGTCAATGTCGACGAGGTGGGAGGCGAA |
| | truncated | GCTCTCGGAAGgtaggctctggtgaccaggattctcaggatttgtggcaccttctgactgt |
| | HBG2 intron 1 | caaactgttcttgtcaatctcacagACTCCTCGTCGTGTATCCCTGGACACA |
| | and full | AAGATTTTTCGAAAGCTTCGGCGACCTCAGCACACCCGACG |
| | length intron | CCGTGATGGGGAATCCCAAAGTCAAAGCCCACGGGAAAAA |
| | 2 (int1-v1) | GGTCCTGGGCGCTTTCTCCGACGGACTCGCCCATCTCGATA |
| | | ATCTGAAAGGAACTTTCGCTACCCTCTCCGAACTCCATTGC |
| | | GATAAACTCCATGTCGACCCCGAAAATTTTAGAgtgagtccaggag |
| | | atgtttcagcactgttgcctttagtctcgaggcaacttagacaactgagtattgatctgagcacagcag |
| | | ggtgtgagctgtttgaagatactggggttgggagtgaagaaactgcagaggactaactgggctgag |
| | | acccagtggcaatgttttagggcctaaggagtgcctctgaaaatctagatggacaactttgactttga |
| | | gaaaagagaggtggaaatgaggaaaatgacttttctttattagatttcggtagaaagaactttcaccttt |
| | | cccctatttttgttattcgttttaaaacatctatctggaggcaggacaagtatggtcattaaaaagatgca |
| | | ggcagaaggcatatattggctcagtcaaagtggggaactttggtggccaaacatacattgctaagg |
| | | ctattcctatatcagctggacacatataaaatgctgctaatgcttcattacaaacttatatcctttaattcc |
| | | agatgggggcaaagtatgtccaggggtgaggaacaattgaaacatttgggctggagtagattttga |
| | | aagtcagctctgtgtgtgtgcgcgcgtgtgtttgtgtgtgtgtgagagcgtgtgtttcttttaacgttttca |
| | | gcctacagcatacagggttcatggtggcaagaagataacaagatttaaattatggccagtgactagt |
| | | gctgcaagaagaacaactacctgcatttaatgggaaagcaaaatctcaggctttgagggaagttaac |
| | | ataggcttgattctgggtggaagcttggtgtgtagttatctggaggccaggctggagctctcagctca |
| | | ctatgggttcatctttattgtctcctttcatctcaacagCTGCTCGGGAATGTCCTCGT |
| | | GTGCGTCCTCGCTCACCATTTCGGGAAGGAGTTTACACCCC |
| | | CCGTCCAAGCCGCTTACCAAAAGGTCGTCGCCGGAGTCGCC |
| | | AACGCTCTCGCTCATAAATACCAT |
| 61 | HBB (CDS | ATGGTCCACCTCACCCCCGAAGAAAAATCCGCAGTCACCGC |
| | diverged with | TCTCTGGGGAAAAGTCAATGTCGACGAGGTGGGAGGCGAA |
| | full length | GCTCTCGGAAGgtaggctctggtgaccaggacaagggagggaaggaaggaccctgtg |
| | HBG2 intron 1 | cctggcaaaagtccaggtcgcttctcaggatttgtggcaccttctgactgtcaaactgttcttgtcaatc |
| | and truncated | tcacagACTCCTCGTCGTGTATCCCTGGACACAAAGATTTTTCG |
| | intron 2 | AAAGCTTCGGCGACCTCAGCACACCCGACGCCGTGATGGGG |
| | (int2-v1) | AATCCCAAAGTCAAAGCCCACGGGAAAAAGGTCCTGGGCG |
| | | CTTTCTCCGACGGACTCGCCCATCTCGATAATCTGAAAGGA |
| | | ACTTTCGCTACCCTCTCCGAACTCCATTGCGATAAACTCCAT |
| | | GTCGACCCCGAAAATTTTAGAgtgagtccaggagatgtttcagcactgttgcctt |
| | | tagtctcgaggcaacttagacaactgagtattgatctgagcacagcagggtgtgagctgtttgaagat |
| | | actggggttgggagtgaagaaactgcagaggactaactgggctgagacccagtggcaatgttttag |
| | | ggcctaaggagtgcctctgaaaatctagatggacaactttgactttgagaaaagagaggtggagct |
| | | ggacacatataaaatgctgctaatgcttcattacaaacttatatcctttaattccagatgggggcaaagt |
| | | atagctctcagctcactatgggttcatctttattgtctcctttcatctcaacagCTGCTCGGGA |
| | | ATGTCCTCGTGTGCGTCCTCGCTCACCATTTCGGGAAGGAG |
| | | TTTACACCCCCCGTCCAAGCCGCTTACCAAAAGGTCGTCGC |
| | | CGGAGTCGCCAACGCTCTCGCTCATAAATACCAT |
| 62 | HBB (CDS | ATGGTCCACCTCACCCCCGAAGAAAAATCCGCAGTCACCGC |
| | diverged with | TCTCTGGGGAAAAGTCAATGTCGACGAGGTGGGAGGCGAA |
| | full length | GCTCTCGGAAGgtaggctctggtgaccaggacaagggagggaaggaaggaccctgtg |
| | HBG2 intron 1 | cctggcaaaagtccaggtcgcttctcaggatttgtggcaccttctgactgtcaaactgttcttgtcaatc |
| | and truncated | tcacagACTCCTCGTCGTGTATCCCTGGACACAAAGATTTTTCG |
| | intron 2 | AAAGCTTCGGCGACCTCAGCACACCCGACGCCGTGATGGGG |
| | (int2-v2) | AATCCCAAAGTCAAAGCCCACGGGAAAAAGGTCCTGGGCG |
| | | CTTTCTCCGACGGACTCGCCCATCTCGATAATCTGAAAGGA |
| | | ACTTTCGCTACCCTCTCCGAACTCCATTGCGATAAACTCCAT |
| | | GTCGACCCCGAAAATTTTAGAgtgagtccaggagatgtttcgctggacacatat |
| | | aaaatgctgctaatgcttcattacaaacttatatcctttaattccagatgggggcaaagtatagctctca |
| | | gctcactatgggttcatctttattgtctcctttcatctcaacagCTGCTCGGGAATGTCCT |
| | | CGTGTGCGTCCTCGCTCACCATTTCGGGAAGGAGTTTACAC |
| | | CCCCCGTCCAAGCCGCTTACCAAAAGGTCGTCGCCGGAGTC |
| | | GCCAACGCTCTCGCTCATAAATACCAT |
| 63 | HBB (CDS | ATGGTCCACCTCACCCCCGAAGAAAAATCCGCAGTCACCGC |
| | diverged with | TCTCTGGGGAAAAGTCAATGTCGACGAGGTGGGAGGCGAA |
| | full length | GCTCTCGGAAGgtaggctctggtgaccaggacaagggagggaaggaaggaccctgtg |
| | HBG2 intron 1 | cctggcaaaagtccaggtcgcttctcaggatttgtggcaccttctgactgtcaaactgttcttgtcaatc |
| | and truncated | tcacagACTCCTCGTCGTGTATCCCTGGACACAAAGATTTTTCG |
| | intron 2 | AAAGCTTCGGCGACCTCAGCACACCCGACGCCGTGATGGGG |
| | (int2-v3) | AATCCCAAAGTCAAAGCCCACGGGAAAAAGGTCCTGGGCG |
| | | CTTTCTCCGACGGACTCGCCCATCTCGATAATCTGAAAGGA |
| | | ACTTTCGCTACCCTCTCCGAACTCCATTGCGATAAACTCCAT |
| | | GTCGACCCCGAAAATTTTAGAgtgagtccaggagatgtttcagcactgttgcctt |
| | | tagtctcgaggcaacttagacaactgagtattgatctgagcacagcagggtgtgagctgtttgaagat |
| | | actggggttgggagtgaagaaactgcagaggactaactgggctgagacccagtggcaagctctca |
| | | gctcactatgggttcatctttattgtctcctttcatctcaacagCTGCTCGGGAATGTCCT |
| | | CGTGTGCGTCCTCGCTCACCATTTCGGGAAGGAGTTTACAC |
| | | CCCCCGTCCAAGCCGCTTACCAAAAGGTCGTCGCCGGAGTC |
| | | GCCAACGCTCTCGCTCATAAATACCAT |
| 64 | HBB (CDS | ATGGTCCACCTCACCCCCGAAGAAAAATCCGCAGTCACCGC |
| | diverged with | TCTCTGGGGAAAAGTCAATGTCGACGAGGTGGGAGGCGAA |
| | truncated | GCTCTCGGAAGgtaggctctggtgaccaggattctcaggatttgtggcaccttctgactgt |
| | HBG2 intron 1 | caaactgttcttgtcaatctcacagACTCCTCGTCGTGTATCCCTGGACACA |
| | and truncated | AAGATTTTTCGAAAGCTTCGGCGACCTCAGCACACCCGACG |
| | intron 2 | CCGTGATGGGGAATCCCAAAGTCAAAGCCCACGGGAAAAA |
| | (int1-v1 + | GGTCCTGGGCGCTTTCTCCGACGGACTCGCCCATCTCGATA |
| | int2-v1) | ATCTGAAAGGAACTTTCGCTACCCTCTCCGAACTCCATTGC |
| | | GATAAACTCCATGTCGACCCCGAAAATTTTAGAgtgagtccaggag |
| | | atgtttcagcactgttgcctttagtctcgaggcaacttagacaactgagtattgatctgagcacagcag |
| | | ggtgtgagctgtttgaagatactggggttgggagtgaagaaactgcagaggactaactgggctgag |
| | | acccagtggcaatgttttagggcctaaggagtgcctctgaaaatctagatggacaactttgactttga |
| | | gaaaagagaggtggagctggacacatataaaatgctgctaatgcttcattacaaacttatatcctttaa |
| | | ttccagatgggggcaaagtatagctctcagctcactatgggttcatctttattgtctcctttcatctcaac |
| | | agCTGCTCGGGAATGTCCTCGTGTGCGTCCTCGCTCACCATT |
| | | TCGGGAAGGAGTTTACACCCCCCGTCCAAGCCGCTTACCAA |
| | | AAGGTCGTCGCCGGAGTCGCCAACGCTCTCGCTCATAAATA |
| | | CCAT |
| 65 | HBB (CDS | ATGGTCCACCTCACCCCCGAAGAAAAATCCGCAGTCACCGC |
| | diverged with | TCTCTGGGGAAAAGTCAATGTCGACGAGGTGGGAGGCGAA |
| | truncated | GCTCTCGGAAGgtaggctctggtgaccaggattctcaggatttgtggcaccttctgactgt |
| | HBG2 intron 1 | caaactgttcttgtcaatctcacagaCTCCTCGTCGTGTATCCCTGGACACA |
| | and truncated | AAGATTTTTCGAAAGCTTCGGCGACCTCAGCACACCCGACG |
| | intron 2 | CCGTGATGGGGAATCCCAAAGTCAAAGCCCACGGGAAAAA |
| | (int1-v1 | GGTCCTGGGCGCTTTCTCCGACGGACTCGCCCATCTCGATA |
| | + int2-v2) | ATCTGAAAGGAACTTTCGCTACCCTCTCCGAACTCCATTGC |
| | | GATAAACTCCATGTCGACCCCGAAAATTTTAGAGtgagtccagga |
| | | gatgtttcgctggacacatataaaatgctgctaatgcttcattacaaacttatatcctttaattccagatg |
| | | ggggcaaagtatagctctcagctcactatgggttcatctttattgtctcctttcatctcaacagCTGC |
| | | TCGGGAATGTCCTCGTGTGCGTCCTCGCTCACCATTTCGGGA |
| | | AGGAGTTTACACCCCCCGTCCAAGCCGCTTACCAAAAGGTC |
| | | GTCGCCGGAGTCGCCAACGCTCTCGCTCATAAATACCAT |
| 66 | HBB (CDS | ATGGTCCACCTCACCCCCGAAGAAAAATCCGCAGTCACCGC |
| | diverged with | TCTCTGGGGAAAAGTCAATGTCGACGAGGTGGGAGGCGAA |
| | truncated | GCTCTCGGAAGgtaggctctggtgaccaggattctcaggatttgtggcaccttctgactgt |
| | HBG2 intron 1 | caaactgttcttgtcaatctcacagACTCCTCGTCGTGTATCCCTGGACACA |
| | and truncated | AAGATTTTTCGAAAGCTTCGGCGACCTCAGCACACCCGACG |
| | intron 2 | CCGTGATGGGGAATCCCAAAGTCAAAGCCCACGGGAAAAA |
| | (int1-v1 | GGTCCTGGGCGCTTTCTCCGACGGACTCGCCCATCTCGATA |
| | + int2-v3) | ATCTGAAAGGAACTTTCGCTACCCTCTCCGAACTCCATTGC |
| | | GATAAACTCCATGTCGACCCCGAAAATTTTAGAgtgagtccaggag |
| | | atgtttcagcactgttgcctttagtctcgaggcaacttagacaactgagtattgatctgagcacagcag |
| | | ggtgtgagctgtttgaagatactggggttgggagtgaagaaactgcagaggactaactgggctgag |
| | | acccagtggcaagctctcagctcactatgggttcatctttattgtctcctttcatctcaacagCTGCT |
| | | CGGGAATGTCCTCGTGTGCGTCCTCGCTCACCATTTCGGGA |
| | | AGGAGTTTACACCCCCCGTCCAAGCCGCTTACCAAAAGGTC |
| | | GTCGCCGGAGTCGCCAACGCTCTCGCTCATAAATACCAT |
| 67 | β- | ATGGTCCACCTCACCCCCGAAGAAAAATCCGCAGTCACCGC |
| | HBB^{div}HBG2^{intr} | TCTCTGGGGAAAAGTCAATGTCGACGAGGTGGGAGGCGAA |
| | Donor | GCTCTCGGAAGgtaggctctggtgaccaggacaagggagggaaggaaggaccctgtg |
| | sequence | cctggcaaaagtccaggtcgcttctcaggatttgtggcaccttctgactgtcaaactgttcttgtcaatc |
| | | tcacagACTCCTCGTCGTGTATCCCTGGACACAAAGATTTTTCG |
| | | AAAGCTTCGGCGACCTCAGCACACCCGACGCCGTGATGGGG |
| | | AATCCCAAAGTCAAAGCCCACGGGAAAAAGGTCCTGGGCG |
| | | CTTTCTCCGACGGACTCGCCCATCTCGATAATCTGAAAGGA |
| | | ACTTTCGCTACCCTCTCCGAACTCCATTGCGATAAACTCCAT |
| | | GTCGACCCCGAAAATTTTAGAgtgagtccaggagatgtttcagcactgttgcctt |
| | | tagtctcgaggcaacttagacaactgagtattgatctgagcacagcagggtgtgagctgtttgaagat |
| | | actggggttgggagtgaagaaactgcagaggactaactgggctgagacccagtggcaatgttttag |
| | | ggcctaaggagtgcctctgaaaatctagatggacaactttgactttgagaaaagagaggtggaaat |
| | | gaggaaaatgacttttctttattagatttcggtagaaagaactttcacctttcccctatttttgttattcgtttt |
| | | aaaacatctatctggaggcaggacaagtatggtcattaaaaagatgcaggcagaaggcatatattg |
| | | gctcagtcaaagtggggaactttggtggccaaacatacattgctaaggctattcctatatcagctgga |
| | | cacatataaaatgctgctaatgcttcattacaaacttatatcctttaattccagatgggggcaaagtatgt |
| | | ccaggggtgaggaacaattgaaacatttgggctggagtagattttgaaagtcagctctgtgtgtgtgt |
| | | gtgtgtgtgtgcgcgcgtgtgtttgtgtgtgtgtgagagcgtgtgtttcttttaacgttttcagcctacag |
| | | catacagggttcatggtggcaagaagataacaagatttaaattatggccagtgactagtgctgcaag |
| | | aagaacaactacctgcatttaatgggaaagcaaaatctcaggctttgagggaagttaacataggctt |
| | | gattctgggtggaagcttggtgtgtagttatctggaggccaggctggagctctcagctcactatgggt |
| | | tcatctttattgtctcctttcatctcaacagCTGCTCGGGAATGTCCTCGTGTGC |
| | | GTCCTCGCTCACCATTTCGGGAAGGAGTTTACACCCCCCGT |
| | | CCAAGCCGCTTACCAAAAGGTCGTCGCCGGAGTCGCCAACG |
| | | CTCTCGCTCATAAATACCATTAA |
| 68 | β- | ATGGTCCACCTCACCCCCGAAGAAAAATCCGCAGTCACCGC |
| | HBB^{div}HBG2^{i2v2} | TCTCTGGGGAAAAGTCAATGTCGACGAGGTGGGAGGCGAA |
| | Donor | GCTCTCGGAAGGTAGGCTCTGGTGACCAGGACAAGGGAGG |
| | sequence | GAAGGAAGGACCCTGTGCCTGGCAAAAGTCCAGGTCGCTTC |
| | | TCAGGATTTGTGGCACCTTCTGACTGTCAAACTGTTCTTGTC |
| | | AATCTCACAGACTCCTCGTCGTGTATCCCTGGACACAAAGA |
| | | TTTTTCGAAAGCTTCGGCGACCTCAGCACACCCGACGCCGT |
| | | GATGGGGAATCCCAAAGTCAAAGCCCACGGGAAAAAGGTC |
| | | CTGGGCGCTTTCTCCGACGGACTCGCCCATCTCGATAATCTG |
| | | AAAGGAACTTTCGCTACCCTCTCCGAACTCCATTGCGATAA |
| | | ACTCCATGTCGACCCCGAAAATTTTAGAGTGAGTCCAGGAG |
| | | ATGTTTCGCTGGACACATATAAAATGCTGCTAATGCTTCATT |
| | | ACAAACTTATATCCTTTAATTCCAGATGGGGGCAAAGTATA |
| | | GCTCTCAGCTCACTATGGGTTCATCTTTATTGTCTCCTTTCAT |
| | | CTCAACAGCTGCTCGGGAATGTCCTCGTGTGCGTCCTCGCTC |
| | | ACCATTTCGGGAAGGAGTTTACACCCCCCGTCCAAGCCGCT |
| | | TACCAAAAGGTCGTCGCCGGAGTCGCCAACGCTCTCGCTCA |
| | | TAAATACCATTAA |
| 69 | 5′ Homology | TAACCTCCTATTTGACACCACTGATTACCCCATTGATAGTCA |
| | arm for β- | CACTTTGGGTTGTAAGTGACTTTTTATTTATTTGTATTTTTGA |
| | HBB^{div}HBG2^{i2v2} | CTGCATTAAGAGGTCTCTAGTTTTTTACCTCTTGTTTCCCAA |
| | construct | AACCTAATAAGTAACTAATGCACAGAGCACATTGATTTGTA |
| | | TTTATTCTATTTTTAGACATAATTTATTAGCATGCATGAGCA |
| | | AATTAAGAAAAACAACAACAAATGAATGCATATATATGTAT |
| | | ATGTATGTGTGTACATATACACATATATATATATATTTTTTT |
| | | TCTTTTCTTACCAGAAGGTTTTAATCCAAATAAGGAGAAGA |
| | | TATGCTTAGAACTGAGGTAGAGTTTTCATCCATTCTGTCCTG |
| | | TAAGTATTTTGCATATTCTGGAGACGCAGGAAGAGATCCAT |
| | | CTACATATCCCAAAGCTGAATTATGGTAGACAAAACTCTTC |
| | | CACTTTTAGTGCATCAATTTCTTATTTGTGTAATAAGAAAAT |
| | | TGGGAAAACGATCTTCAATATGCTTACCAAGCTGTGATTCC |
| | | AAATATTACGTAAATACACTTGCAAAGGAGGATGTTTTTAG |
| | | TAGCAATTTGTACTGATGGTATGGGGCCAAGAGATATATCT |
| | | TAGAGGGAGGGCTGAGGGTTTGAAGTCCAACTCCTAAGCCA |
| | | GTGCCAGAAGAGCCAAGGACAGGTACGGCTGTCATCACTTA |
| | | GACCTCACCCTGTGGAGCCACACCCTAGGGTTGGCCAATCT |
| | | ACTCCCAGGAGCAGGGAGGGCAGGAGCCAGGGCTGGGCAT |
| | | AAAAGTCAGGGCAGAGCCATCTATTGCTTACATTTGCTTCT |
| | | GACACAACTGTGTTCACTAGCAACCTCAAACAGACACC |
| 70 | 3′ Homology | CTGCCCTGTGGGGCAAGGTGAACGTGGATGAAGTTGGTGGT |
| | arm for 3- | GAGGCCCTGGGCAGgttggtatcaaggttacaagacaggtttaaggagaccaatagaa |
| | HBB^{div}HBG2^{i2v2} | actgggcatgtggagacagagaagactcttgggtttctgataggcactgactctctctgcctattggt |
| | construct | ctattttcccacccttagGCTGCTGGTGGTCTACCCTTGGACCCAGAGG |
| | | ttctttgagtcctttggggatctgtccactcctgatgctgtt |
| | | ATGGGCAACCCTAAGGTGAAGGCTCATGGCAAGAAAGTGC |
| | | TCGGTGCCTTTAGTGATGGCCTGGCTCACCTGGACAACCTC |
| | | AAGGGCACCTTTGCCACACTGAGTGAGCTGCACTGTGACAA |
| | | GCTGCACGTGGATCCTGAGAACTTCAGGgtgagtctatgggacccttgat |
| | | gttttctttccccttcttttctatggttaagttcatgtcataggaaggggataagtaacagggtacagttta |
| | | gaatgggaaacagacgaatgattgcatcagtgtggaagtctcaggatcgttttagtttcttttatttgct |
| | | gttcataacaattgttttcttttgtttaattcttgctttctttttttttcttctccgcaatttttactattatacttaat |
| | | gccttaacattgtgtataacaaaaggaaatatctctgagatacattaagtaacttaaaaaaaaactttac |
| | | acagtctgcctagtacattactatttggaatatatgtgtgcttatttgcatattcataatctccctactttat |
| 71 | WT AAT | ATGCCGTCTTCTGTCTCGTGGGGCATCCTCCTGCTGGCAGGC |
| | cDNA | CTGTGCTGCCTGGTCCCTGTCTCCCTGGCTGAGGATCCCCAG |
| | | GGAGATGCTGCCCAGAAGACAGATACATCCCACCATGATCA |
| | | GGATCACCCAACCTTCAACAAGATCACCCCCAACCTGGCTG |
| | | AGTTCGCCTTCAGCCTATACCGCCAGCTGGCACACCAGTCC |
| | | AACAGCACCAATATCTTCTTCTCCCCAGTGAGCATCGCTAC |
| | | AGCCTTTGCAATGCTCTCCCTGGGGACCAAGGCTGACACTC |
| | | ACGATGAAATCCTGGAGGGCCTGAATTTCAACCTCACGGAG |
| | | ATTCCGGAGGCTCAGATCCATGAAGGCTTCCAGGAACTCCT |
| | | CCGTACCCTCAACCAGCCAGACAGCCAGCTCCAGCTGACCA |
| | | CCGGCAATGGCCTGTTCCTCAGCGAGGGCCTGAAGCTAGTG |
| | | GATAAGTTTTTGGAGGATGTTAAAAAGTTGTACCACTCAGA |
| | | AGCCTTCACTGTCAACTTCGGGGACACCGAAGAGGCCAAGA |
| | | AACAGATCAACGATTACGTGGAGAAGGGTACTCAAGGGAA |
| | | AATTGTGGATTTGGTCAAGGAGCTTGACAGAGACACAGTTT |
| | | TTGCTCTGGTGAATTACATCTTCTTTAAAGGCAAATGGGAG |
| | | AGACCCTTTGAAGTCAAGGACACCGAGGAAGAGGACTTCC |
| | | ACGTGGACCAGGTGACCACCGTGAAGGTGCCTATGATGAA |
| | | GCGTTTAGGCATGTTTAACATCCAGCACTGTAAGAAGCTGT |
| | | CCAGCTGGGTGCTGCTGATGAAATACCTGGGCAATGCCACC |
| | | GCCATCTTCTTCCTGCCTGATGAGGGGAAACTACAGCACCT |
| | | GGAAAATGAACTCACCCACGATATCATCACCAAGTTCCTGG |
| | | AAAATGAAGACAGAAGGTCTGCCAGCTTACATTTACCCAAA |
| | | CTGTCCATTACTGGAACCTATGATCTGAAGAGCGTCCTGGG |
| | | TCAACTGGGCATCACTAAGGTCTTCAGCAATGGGGCTGACC |
| | | TCTCCGGGGTCACAGAGGAGGCACCCCTGAAGCTCTCCAAG |
| | | GCCGTGCATAAGGCTGTGCTGACCATCGACGAGAAAGGGA |
| | | CTGAAGCTGCTGGGGCCATGTTTTTAGAGGCCATACCCATG |
| | | TCTATCCCCCCCGAGGTCAAGTTCAACAAACCCTTTGTCTTC |
| | | TTAATGATTGAACAAAATACCAAGTCTCCCCTCTTCATGGG |
| | | AAAAGTGGTGAATCCCACCCAAAAA |
| 72 | AAT cDNA | ATGCCGTCTTCTGTCTCGTGGGGCATCCTCCTGCTGGCAGGC |
| | with HBA1 | CTGTGCTGCCTGGTCCCTGTCTCCCTGGCTGAGGATCCCCAG |
| | introns | GGAGATGCTGCCCAGAAGACAGATACATCCCACCATGATCA |
| | | GGATCACCCAACCTTCAACAAGATCACCCCCAACCTGGCTG |
| | | AGTTCGCCTTCAGCCTATACCGCCAGCTGGCACACCAGTCC |
| | | AACAGCACCAATATCTTCTTCTCCCCAGTGAGCATCGCTAC |
| | | AGCCTTTGCAATGCTCTCCCTGGGGACCAAGGCTGACACTC |
| | | ACGATGAAATCCTGGAGGGCCTGAATTTCAACCTCACGGAG |
| | | ATTCCGGAGGCTCAGATCCATGAAGGCTTCCAGGAACTCCT |
| | | CCGTACCCTCAACCAGCCAGACAGCCAGCTCCAGCTGACCA |
| | | CCGGCAATGGCCTGTTCCTCAGCGAGGGCCTGAAGCTAGTG |
| | | GATAAGTTTTTGGAGGATGTTAAAAAGTTGTACCACTCAGA |
| | | AGCCTTCACTGTCAACTTCGGGGACACCGAAGAGGCCAAGA |
| | | AACAGATCAACGATTACGTGGAGAAGGGTACTCAAGGGAA |
| | | AATTGTGGATTTGGTCAAGGAGCTTGACAGAGACACAGTTT |
| | | TTGCTCTGGTGAATTACATCTTCTTTAAAGgtgaggctccctcccctgct |
| | | ccgacccgggctcctcgcccgcccggacccacaggccaccctcaaccgtcctggccccggacc |
| | | caaaccccacccctcactctgcttctccccgcagGCAAATGGGAGAGACCCTTT |
| | | GAAGTCAAGGACACCGAGGAAGAGGACTTCCACGTGGACC |
| | | AGGTGACCACCGTGAAGGTGCCTATGATGAAGCGTTTAGGC |
| | | ATGTTTAACATCCAGCACTGTAAGAAGCTGTCCAGCTGGGT |
| | | GCTGCTGATGAAATACCTGGGCAATGCCACCGCCATCTTCT |
| | | TCCTGCCTGATGAGGGGAAACTACAGCACCTGGAAAATGA |
| | | ACTCACCCACGATATCATCACCAAGTTCCTGGAAAATGAAG |
| | | ACAGAAGgtgagcggcgggccgggagcgatctgggtcgaggggcgagatggcgccttc |
| | | ctcgcagggcagaggatcacgcgggttgcgggaggtgtagcgcaggcggcggctgcgggcct |
| | | gggccctcggccccactgaccctcttctctgcacagGTCTGCCAGCTTACATTTA |
| | | CCCAAACTGTCCATTACTGGAACCTATGATCTGAAGAGCGT |
| | | CCTGGGTCAACTGGGCATCACTAAGGTCTTCAGCAATGGGG |
| | | CTGACCTCTCCGGGGTCACAGAGGAGGCACCCCTGAAGCTC |
| | | TCCAAGGCCGTGCATAAGGCTGTGCTGACCATCGACGAGAA |
| | | AGGGACTGAAGCTGCTGGGGCCATGTTTTTAGAGGCCATAC |
| | | CCATGTCTATCCCCCCCGAGGTCAAGTTCAACAAACCCTTT |
| | | GTCTTCTTAATGATTGAACAAAATACCAAGTCTCCCCTCTTC |
| | | ATGGGAAAAGTGGTGAATCCCACCCAAAAA |
| 73 | AAT cDNA | ATGCCGTCTTCTGTCTCGTGGGGCATCCTCCTGCTGGCAGGC |
| | with HBG2 | CTGTGCTGCCTGGTCCCTGTCTCCCTGGCTGAGGATCCCCAG |
| | introns | GGAGATGCTGCCCAGAAGACAGATACATCCCACCATGATCA |
| | | GGATCACCCAACCTTCAACAAGATCACCCCCAACCTGGCTG |
| | | AGTTCGCCTTCAGCCTATACCGCCAGCTGGCACACCAGTCC |
| | | AACAGCACCAATATCTTCTTCTCCCCAGTGAGCATCGCTAC |
| | | AGCCTTTGCAATGCTCTCCCTGGGGACCAAGGCTGACACTC |
| | | ACGATGAAATCCTGGAGGGCCTGAATTTCAACCTCACGGAG |
| | | ATTCCGGAGGCTCAGATCCATGAAGGCTTCCAGGAACTCCT |
| | | CCGTACCCTCAACCAGCCAGACAGCCAGCTCCAGCTGACCA |
| | | CCGGCAATGGCCTGTTCCTCAGCGAGGGCCTGAAGCTAGTG |
| | | GATAAGTTTTTGGAGGATGTTAAAAAGTTGTACCACTCAGA |
| | | AGCCTTCACTGTCAACTTCGGGGACACCGAAGAGGCCAAGA |
| | | AACAGATCAACGATTACGTGGAGAAGGGTACTCAAGGGAA |
| | | AATTGTGGATTTGGTCAAGGAGCTTGACAGAGACACAGTTT |
| | | TTGCTCTGGTGAATTACATCTTCTTTAAAGgtaggctctggtgaccagg |
| | | acaagggagggaaggaaggaccctgtgcctggcaaaagtccaggtcgcttctcaggatttgtggc |
| | | accttctgactgtcaaactgttcttgtcaatctcacagGCAAATGGGAGAGACCCTT |
| | | TGAAGTCAAGGACACCGAGGAAGAGGACTTCCACGTGGAC |
| | | CAGGTGACCACCGTGAAGGTGCCTATGATGAAGCGTTTAGG |
| | | CATGTTTAACATCCAGCACTGTAAGAAGCTGTCCAGCTGGG |
| | | TGCTGCTGATGAAATACCTGGGCAATGCCACCGCCATCTTC |
| | | TTCCTGCCTGATGAGGGGAAACTACAGCACCTGGAAAATGA |
| | | ACTCACCCACGATATCATCACCAAGTTCCTGGAAAATGAAG |
| | | ACAGAAGgtgagtccaggagatgtttcagcactgttgcctttagtctcgaggcaacttagaca |
| | | actgagtattgatctgagcacagcagggtgtgagctgtttgaagatactggggttgggagtgaagaa |
| | | actgcagaggactaactgggctgagacccagtggcaatgttttagggcctaaggagtgcctctgaa |
| | | aatctagatggacaactttgactttgagaaaagagaggtggaaatgaggaaaatgacttttctttatta |
| | | gatttcggtagaaagaactttcacctttcccctatttttgttattcgttttaaaacatctatctggaggcag |
| | | gacaagtatggtcattaaaaagatgcaggcagaaggcatatattggctcagtcaaagtggggaactt |
| | | tggtggccaaacatacattgctaaggctattcctatatcagctggacacatataaaatgctgctaatgc |
| | | ttcattacaaacttatatcctttaattccagatgggggcaaagtatgtccaggggtgaggaacaattga |
| | | aacatttgggctggagtagattttgaaagtcagctctgtgtgtgtgtgtgtgtgtgtgcgcgcgtgtgtt |
| | | tgtgtgtgtgtgagagcgtgtgtttcttttaacgttttcagcctacagcatacagggttcatggtggcaa |
| | | gaagataacaagatttaaattatggccagtgactagtgctgcaagaagaacaactacctgcatttaat |
| | | gggaaagcaaaatctcaggctttgagggaagttaacataggcttgattctgggtggaagcttggtgt |
| | | gtagttatctggaggccaggctggagctctcagctcactatgggttcatctttattgtctcctttcatctc |
| | | aacagGTCTGCCAGCTTACATTTACCCAAACTGTCCATTACTG |
| | | GAACCTATGATCTGAAGAGCGTCCTGGGTCAACTGGGCATC |
| | | ACTAAGGTCTTCAGCAATGGGGCTGACCTCTCCGGGGTCAC |
| | | AGAGGAGGCACCCCTGAAGCTCTCCAAGGCCGTGCATAAG |
| | | GCTGTGCTGACCATCGACGAGAAAGGGACTGAAGCTGCTG |
| | | GGGCCATGTTTTTAGAGGCCATACCCATGTCTATCCCCCCCG |
| | | AGGTCAAGTTCAACAAACCCTTTGTCTTCTTAATGATTGAAC |
| | | AAAATACCAAGTCTCCCCTCTTCATGGGAAAAGTGGTGAAT |
| | | CCCACCCAAAAA |
| 74 | HBG2 intron | gtaggctctggtgaccaggacaagggagggaaggaaggaccctgtgcctggcaaaagtccaggt |
| | 1-WT | cgcttctcaggatttgtggcaccttctgactgtcaaactgttcttgtcaatctcacag |
| 75 | HBG2 Intron | gtaggctctggtgaccaggattctcaggatttgtggcaccttctgactgtcaaactgttcttgtcaatct |
| | 1-modification | cacag |
| | 1 (int1-v1) | |
| 76 | HBG2 intron | gtgagtccaggagatgtttcagcactgttgcctttagtctcgaggcaacttagacaactgagtattgat |
| | 2-WT | ctgagcacagcagggtgtgagctgtttgaagatactggggttgggagtgaagaaactgcagagga |
| | | ctaactgggctgagacccagtggcaatgttttagggcctaaggagtgcctctgaaaatctagatgga |
| | | caactttgactttgagaaaagagaggtggaaatgaggaaaatgacttttctttattagatttcggtaga |
| | | aagaactttcacctttcccctatttttgttattcgttttaaaacatctatctggaggcaggacaagtatggt |
| | | cattaaaaagatgcaggcagaaggcatatattggctcagtcaaagtggggaactttggtggccaaa |
| | | catacattgctaaggctattcctatatcagctggacacatataaaatgctgctaatgcttcattacaaac |
| | | ttatatcctttaattccagatgggggcaaagtatgtccaggggtgaggaacaattgaaacatttgggc |
| | | tggagtagattttgaaagtcagctctgtgtgtgtgtgtgtgtgtgtgcgcgcgtgtgtttgtgtgtgtgtg |
| | | agagcgtgtgtttcttttaacgttttcagcctacagcatacagggttcatggtggcaagaagataacaa |
| | | gatttaaattatggccagtgactagtgctgcaagaagaacaactacctgcatttaatgggaaagcaa |
| | | aatctcaggctttgagggaagttaacataggcttgattctgggtggaagcttggtgtgtagttatctgg |
| | | aggccaggctggagctctcagctcactatgggttcatctttattgtctcctttcatctcaacag |
| 77 | HBG2 Intron | gtgagtccaggagatgtttcagcactgttgcctttagtctcgaggcaacttagacaactgagtattgat |
| | 2- | ctgagcacagcagggtgtgagctgtttgaagatactggggttgggagtgaagaaactgcagagga |
| | modification | ctaactgggctgagacccagtggcaatgttttagggcctaaggagtgcctctgaaaatctagatgga |
| | 1 (int2-v1) | caactttgactttgagaaaagagaggtggagctggacacatataaaatgctgctaatgcttcattaca |
| | | aacttatatcctttaattccagatgggggcaaagtatagctctcagctcactatgggttcatctttattgt |
| | | ctcctttcatctcaacag |
| 78 | HBG2 Intron | gtgagtccaggagatgtttcgctggacacatataaaatgctgctaatgcttcattacaaacttatatcct |
| | 2- | ttaattccagatgggggcaaagtatagctctcagctcactatgggttcatctttattgtctcctttcatctc |
| | modification | aacag |
| | 2 (int2-v2) | |
| 79 | HBG2 Intron | gtgagtccaggagatgtttcagcactgttgcctttagtctcgaggcaacttagacaactgagtattgat |
| | 2- | ctgagcacagcagggtgtgagctgtttgaagatactggggttgggagtgaagaaactgcagagga |
| | modification | ctaactgggctgagacccagtggcaagctctcagctcactatgggttcatctttattgtctcctttcatct |
| | 3 (int2-v3) | caacag |
| 80 | E6V corrective | GAGGAAAAATCCGCAGTCA |
| | SNP donor | |
| | sequence | |
| 81 | Human HBB | MVHLTPEEKSAVTALWGKVNVDEVGGEALGRLLVVYPWTQR |
| | amino acid | FFESFGDLSTPDAVMGNPKVKAHGKKVLGAFSDGLAHLDNLK |
| | sequence | GTFATLSELHCDKLHVDPENFRLLGNVLVCVLAHHFGKEFTPP |
| | | VQAAYQKVVAGVANALAHKYH |
| 82 | HBB exon 1 | MVHLTPEEKSAVTALWGKVNVDEVGGEALG |
| | amino acid | |
| | sequence | |
| 83 | HBB exon 2 | LLVVYPWTQRFFESFGDLSTPDAVMGNPKVKAHGKKVLGAFS |
| | amino acid | DGLAHLDNLKGTFATLSELHCDKLHVDPENFR |
| | sequence | |
| 84 | HBB exon 3 | LLGNVLVCVLAHHFGKEFTPPVQAAYQKVVAGVANALAHKY |
| | amino acid | H |
| | sequence | |
| 85 | WT HBB exon 2 | GCTGCTGGTGGTCTACCCTTGGACCCAGAGGTTCTTTGAGTC |
| | nucleotide | CTTTGGGGATCTGTCCACTCCTGATGCTGTTATGGGCAACCC |
| | sequence | TAAGGTGAAGGCTCATGGCAAGAAAGTGCTCGGTGCCTTTA |
| | | GTGATGGCCTGGCTCACCTGGACAACCTCAAGGGCACCTTT |
| | | GCCACACTGAGTGAGCTGCACTGTGACAAGCTGCACGTGGA |
| | | TCCTGAGAACTTCAGG |
| 86 | WT HBB exon 3 | CTCCTGGGCAACGTGCTGGTCTGTGTGCTGGCCCATCACTTT |
| | nucleotide | GGCAAAGAATTCACCCCACCAGTGCAGGCTGCCTATCAGAA |
| | sequence | AGTGGTGGCTGGTGTGGCTAATGCCCTGGCCCACAAGTATC |
| | | AC |
| 87 | WT HBB exon | ATGGTGCATCTGACTCCTGAGGAGAAGTCTGCCGTTACTGC |
| | and intron | CCTGTGGGGCAAGGTGAACGTGGATGAAGTTGGTGGTGAG |
| | sequence | GCCCTGGGCAGgttggtatcaaggttacaagacaggtttaaggagaccaatagaaactgg |
| | | gcatgtggagacagagaagactcttgggtttctgataggcactgactctctctgcctattggtctatttt |
| | | cccacccttagGCTGCTGGTGGTCTACCCTTGGACCCAGAGGTTCT |
| | | TTGAGTCCTTTGGGGATCTGTCCACTCCTGATGCTGTTATGG |
| | | GCAACCCTAAGGTGAAGGCTCATGGCAAGAAAGTGCTCGG |
| | | TGCCTTTAGTGATGGCCTGGCTCACCTGGACAACCTCAAGG |
| | | GCACCTTTGCCACACTGAGTGAGCTGCACTGTGACAAGCTG |
| | | CACGTGGATCCTGAGAACTTCAGGgtgagtctatgggacgcttgatgttttctt |
| | | tccccttcttttctatggttaagttcatgtcataggaaggggataagtaacagggtacagtttagaatgg |
| | | gaaacagacgaatgattgcatcagtgtggaagtctcaggatcgttttagtttcttttatttgctgttcata |
| | | acaattgttttcttttgtttaattcttgctttctttttttttcttctccgcaatttttactattatacttaatgccttaa |
| | | cattgtgtataacaaaaggaaatatctctgagatacattaagtaacttaaaaaaaaactttacacagtct |
| | | gcctagtacattactatttggaatatatgtgtgcttatttgcatattcataatctccctactttattttcttttatt |
| | | tttaattgatacataatcattatacatatttatgggttaaagtgtaatgttttaatatgtgtacacatattgac |
| | | caaatcagggtaattttgcatttgtaattttaaaaaatgctttcttcttttaatatacttttttgtttatcttatttc |
| | | taatactttccctaatctctttctttcagggcaataatgatacaatgtatcatgcctctttgcaccattctaa |
| | | agaataacagtgataatttctgggttaaggcaatagcaatatctctgcatataaatatttctgcatataaa |
| | | ttgtaactgatgtaagaggtttcatattgctaatagcagctacaatccagctaccattctgcttttattttat |
| | | ggttgggataaggctggattattctgagtccaagctaggcccttttgctaatcatgttcatacctcttatc |
| | | ttcctcccacagCTCCTGGGCAACGTGCTGGTCTGTGTGCTGGCCC |
| | | ATCACTTTGGCAAAGAATTCACCCCACCAGTGCAGGCTGCC |
| | | TATCAGAAAGTGGTGGCTGGTGTGGCTAATGCCCTGGCCCA |
| | | CAAGTATCAC |
| 88 | β- | ATGGTcCAcCTcACcCCcGAaGAaAAaTCcGCaGTcACcGCTCTC |
| | HBB^{div}HBG2^{intr}- | TGGGGAAAAGTCAATGTCGACGAGGTGGGAGGCGAAGCTC |
| | bGH donor | TCGGAAGgtaggctctggtgaccaggacaagggagggaaggaaggaccctgtgcctggc |
| | sequence | aaaagtccaggtcgcttctcaggatttgtggcaccttctgactgtcaaactgttcttgtcaatctcacag |
| | | ACTCCTCGTCGTGTATCCCTGGACACAAAGATTTTTCGAAA |
| | | GCTTCGGCGACCTCAGCACACCCGACGCCGTGATGGGGAAT |
| | | CCCAAAGTCAAAGCCCACGGGAAAAAGGTCCTGGGCGCTTT |
| | | CTCCGACGGACTCGCCCATCTCGATAATCTGAAAGGAACTT |
| | | TCGCTACCCTCTCCGAACTCCATTGCGATAAACTCCATGTCG |
| | | ACCCCGAAAATTTTAGAgtgagtccaggagatgtttcagcactgttgcctttagtctc |
| | | gaggcaacttagacaactgagtattgatctgagcacagcagggtgtgagctgtttgaagatactggg |
| | | gttgggagtgaagaaactgcagaggactaactgggctgagacccagtggcaatgttttagggccta |
| | | aggagtgcctctgaaaatctagatggacaactttgactttgagaaaagagaggtggaaatgaggaa |
| | | aatgacttttctttattagatttcggtagaaagaactttcacctttcccctatttttgttattcgttttaaaaca |
| | | tctatctggaggcaggacaagtatggtcattaaaaagatgcaggcagaaggcatatattggctcagt |
| | | caaagtggggaactttggtggccaaacatacattgctaaggctattcctatatcagctggacacatat |
| | | aaaatgctgctaatgcttcattacaaacttatatcctttaattccagatgggggcaaagtatgtccagg |
| | | ggtgaggaacaattgaaacatttgggctggagtagattttgaaagtcagctctgtgtgtgtgtgtgtgt |
| | | gtgtgcgcgcgtgtgtttgtgtgtgtgtgagagcgtgtgtttcttttaacgttttcagcctacagcataca |
| | | gggttcatggtggcaagaagataacaagatttaaattatggccagtgactagtgctgcaagaagaac |
| | | aactacctgcatttaatgggaaagcaaaatctcaggctttgagggaagttaacataggcttgattctg |
| | | ggtggaagcttggtgtgtagttatctggaggccaggctggagctctcagctcactatgggttcatcttt |
| | | attgtctcctttcatctcaacagCTGCTCGGGAATGTCCTCGTGTGCGTCCT |
| | | CGCTCACCATTTCGGGAAGGAGTTTACACCCCCCGTCCAAG |
| | | CCGCTTACCAAAAGGTCGTCGCCGGAGTCGCCAACGCTCTC |
| | | GCTCATAAATACCATTAACTGTGCCTTCTAGTTGCCAGCCAT |
| | | CTGTTGTTTGCCCCTCCCCCGTGCCTTCCTTGACCCTGGAAG |
| | | GTGCCACTCCCACTGTCCTTTCCTAATAAAATGAGGAAATT |
| | | GCATCGCATTGTCTGAGTAGGTGTCATTCTATTCTGGGGGGT |
| | | GGGGTGGGGCAGGACAGCAAGGGGGAGGATTGGGAAGAC |
| | | AATAGCAGGCATGCTGGGGA |
| 89 | β- | ATGGTcCAcCTcACcCCcGAaGAaAAaTCcGCaGTcACcGCTCTC |
| | HBB^{div}HBG^{i2v2}- | TGGGGAAAAGTCAATGTCGACGAGGTGGGAGGCGAAGCTC |
| | bGH donor | TCGGAAGGTAGGCTCTGGTGACCAGGACAAGGGAGGGAAG |
| | sequence | GAAGGACCCTGTGCCTGGCAAAAGTCCAGGTCGCTTCTCAG |
| | | GATTTGTGGCACCTTCTGACTGTCAAACTGTTCTTGTCAATC |
| | | TCACAGACTCCTCGTCGTGTATCCCTGGACACAAAGATTTTT |
| | | CGAAAGCTTCGGCGACCTCAGCACACCCGACGCCGTGATGG |
| | | GGAATCCCAAAGTCAAAGCCCACGGGAAAAAGGTCCTGGG |
| | | CGCTTTCTCCGACGGACTCGCCCATCTCGATAATCTGAAAG |
| | | GAACTTTCGCTACCCTCTCCGAACTCCATTGCGATAAACTCC |
| | | ATGTCGACCCCGAAAATTTTAGAGTGAGTCCAGGAGATGTT |
| | | TCGCTGGACACATATAAAATGCTGCTAATGCTTCATTACAA |
| | | ACTTATATCCTTTAATTCCAGATGGGGGCAAAGTATAGCTC |
| | | TCAGCTCACTATGGGTTCATCTTTATTGTCTCCTTTCATCTCA |
| | | ACAGCTGCTCGGGAATGTCCTCGTGTGCGTCCTCGCTCACC |
| | | ATTTCGGGAAGGAGTTTACACCCCCCGTCCAAGCCGCTTAC |
| | | CAAAAGGTCGTCGCCGGAGTCGCCAACGCTCTCGCTCATAA |
| | | ATACCATTAACTGTGCCTTCTAGTTGCCAGCCATCTGTTGTT |
| | | TGCCCCTCCCCCGTGCCTTCCTTGACCCTGGAAGGTGCCACT |
| | | CCCACTGTCCTTTCCTAATAAAATGAGGAAATTGCATCGCA |
| | | TTGTCTGAGTAGGTGTCATTCTATTCTGGGGGGTGGGGTGG |
| | | GGCAGGACAGCAAGGGGGAGGATTGGGAAGACAATAGCAG |
| | | GCATGCTGGGGA |
| 90 | β- | ATGGTcCAcCTcACcCCcGAaGAaAAaTCcGCaGTcACcGCTCTC |
| | HBB^{div}HBG2^{intr}- | TGGGGAAAAGTCAATGTCGACGAGGTGGGAGGCGAAGCTC |
| | SV40 donor | TCGGAAGgtaggctctggtgaccaggacaagggagggaaggaaggaccctgtgcctggc |
| | sequence | aaaagtccaggtcgcttctcaggatttgtggcaccttctgactgtcaaactgttcttgtcaatctcacag |
| | | ACTCCTCGTCGTGTATCCCTGGACACAAAGATTTTTCGAAA |
| | | GCTTCGGCGACCTCAGCACACCCGACGCCGTGATGGGGAAT |
| | | CCCAAAGTCAAAGCCCACGGGAAAAAGGTCCTGGGCGCTTT |
| | | CTCCGACGGACTCGCCCATCTCGATAATCTGAAAGGAACTT |
| | | TCGCTACCCTCTCCGAACTCCATTGCGATAAACTCCATGTCG |
| | | ACCCCGAAAATTTTAGAgtgagtccaggagatgtttcagcactgttgcctttagtctc |
| | | gaggcaacttagacaactgagtattgatctgagcacagcagggtgtgagctgtttgaagatactggg |
| | | gttgggagtgaagaaactgcagaggactaactgggctgagacccagtggcaatgttttagggccta |
| | | aggagtgcctctgaaaatctagatggacaactttgactttgagaaaagagaggtggaaatgaggaa |
| | | aatgacttttctttattagatttcggtagaaagaactttcacctttcccctatttttgttattcgttttaaaaca |
| | | tctatctggaggcaggacaagtatggtcattaaaaagatgcaggcagaaggcatatattggctcagt |
| | | caaagtggggaactttggtggccaaacatacattgctaaggctattcctatatcagctggacacatat |
| | | aaaatgctgctaatgcttcattacaaacttatatcctttaattccagatgggggcaaagtatgtccagg |
| | | ggtgaggaacaattgaaacatttgggctggagtagattttgaaagtcagctctgtgtgtgtgtgtgtgt |
| | | gtgtgcgcgcgtgtgtttgtgtgtgtgtgagagcgtgtgtttcttttaacgttttcagcctacagcataca |
| | | gggttcatggtggcaagaagataacaagatttaaattatggccagtgactagtgctgcaagaagaac |
| | | aactacctgcatttaatgggaaagcaaaatctcaggctttgagggaagttaacataggcttgattctg |
| | | ggtggaagcttggtgtgtagttatctggaggccaggctggagctctcagctcactatgggttcatcttt |
| | | attgtctcctttcatctcaacagCTGCTCGGGAATGTCCTCGTGTGCGTCCT |
| | | CGCTCACCATTTCGGGAAGGAGTTTACACCCCCCGTCCAAG |
| | | CCGCTTACCAAAAGGTCGTCGCCGGAGTCGCCAACGCTCTC |
| | | GCTCATAAATACCATTAAaacttgtttattgcagcttataatggttacaaataaagca |
| | | atagcatcacaaatttcacaaataaagcatttttttcactgcattctagttgtggtttgtccaaactcatca |
| | | atgtatctta |
| 91 | β- | ATGGTcCAcCTcACcCCcGAaGAaAAaTCcGCaGTcACcGCTCTC |
| | HBB^{div}HBG2^{i2v2}- | TGGGGAAAAGTCAATGTCGACGAGGTGGGAGGCGAAGCTC |
| | SV40 donor | TCGGAAGGTAGGCTCTGGTGACCAGGACAAGGGAGGGAAG |
| | sequence | GAAGGACCCTGTGCCTGGCAAAAGTCCAGGTCGCTTCTCAG |
| | | GATTTGTGGCACCTTCTGACTGTCAAACTGTTCTTGTCAATC |
| | | TCACAGACTCCTCGTCGTGTATCCCTGGACACAAAGATTTTT |
| | | CGAAAGCTTCGGCGACCTCAGCACACCCGACGCCGTGATGG |
| | | GGAATCCCAAAGTCAAAGCCCACGGGAAAAAGGTCCTGGG |
| | | CGCTTTCTCCGACGGACTCGCCCATCTCGATAATCTGAAAG |
| | | GAACTTTCGCTACCCTCTCCGAACTCCATTGCGATAAACTCC |
| | | ATGTCGACCCCGAAAATTTTAGAGTGAGTCCAGGAGATGTT |
| | | TCGCTGGACACATATAAAATGCTGCTAATGCTTCATTACAA |
| | | ACTTATATCCTTTAATTCCAGATGGGGGCAAAGTATAGCTC |
| | | TCAGCTCACTATGGGTTCATCTTTATTGTCTCCTTTCATCTCA |
| | | ACAGCTGCTCGGGAATGTCCTCGTGTGCGTCCTCGCTCACC |
| | | ATTTCGGGAAGGAGTTTACACCCCCCGTCCAAGCCGCTTAC |
| | | CAAAAGGTCGTCGCCGGAGTCGCCAACGCTCTCGCTCATAA |
| | | ATACCATTAAaacttgtttattgcagcttataatggttacaaataaagcaatagcatcacaaat |
| | | ttcacaaataaagcatttttttcactgcattctagttgtggtttgtccaaactcatcaatgtatctta |
| 92 | Synthetic HBB | cuugccccac agggcaguaa guuuuagagc uagaaauagc aaguuaaaau |
| | MS sgRNA | aaggcuaguc |
| | sequence with | cguuaucaac uugaaaaagu ggcaccgagu cggugcuuuu |
| | modified MS | |
| | nucleotides at | |
| | 1^{st} three | |
| | positions of | |
| | 5′ and 3′ ends | |
| | (see | |
| | annotations | |
| | below) | |

<210> 92
<211> 100

### <212> RNA

### <213> Artificial Sequence

<220>
<223> Synthetic HBB MS sgRNA
<220>
<221> modified base
<222> (1) . . . (3)
<223> Nucleotide with 2′-O-methyl modification and MS Modification in the phosphate backbone
<220>
<221> modified_base
<222> (97) . . . (99)
<223> Nucleotide with 2′-O-methyl modification and MS Modification in the phosphate backbone
<400> 92

| | |
|---|---|
| cuugccccac agggcaguaa guuuuagagc uagaaauagc aaguuaaaau aaggcuaguc | 60 |
| cguuaucaac uugaaaaagu ggcaccgagu cggugcuuuu | 100 |

TABLE 9
Additional Sequence Tables

| | | |
|---|---|---|
| 93 | AAT exon 4 | ATGCCGTCTTCTGTCTCGTGGGGCATCCTCCTGCTGGCAGGC |
| | | CTGTGCTGCCTGGTCCCTGTCTCCCTGGCTGAGGATCCCCAG |
| | | GGAGATGCTGCCCAGAAGACAGATACATCCCACCATGATCA |
| | | GGATCACCCAACCTTCAACAAGATCACCCCCAACCTGGCTG |
| | | AGTTCGCCTTCAGCCTATACCGCCAGCTGGCACACCAGTCC |
| | | AACAGCACCAATATCTTCTTCTCCCCAGTGAGCATCGCTAC |
| | | AGCCTTTGCAATGCTCTCCCTGGGGACCAAGGCTGACACTC |
| | | ACGATGAAATCCTGGAGGGCCTGAATTTCAACCTCACGGAG |
| | | ATTCCGGAGGCTCAGATCCATGAAGGCTTCCAGGAACTCCT |
| | | CCGTACCCTCAACCAGCCAGACAGCCAGCTCCAGCTGACCA |
| | | CCGGCAATGGCCTGTTCCTCAGCGAGGGCCTGAAGCTAGTG |
| | | GATAAGTTTTTGGAGGATGTTAAAAAGTTGTACCACTCAGA |
| | | AGCCTTCACTGTCAACTTCGGGGACACCGAAGAGGCCAAGA |
| | | AACAGATCAACGATTACGTGGAGAAGGGTACTCAAGGGAA |
| | | AATTGTGGATTTGGTCAAGGAGCTTGACAGAGACACAGTTT |
| | | TTGCTCTGGTGAATTACATCTTCTTTAAAG |
| 94 | AAT exon 5 | GCAAATGGGAGAGACCCTTTGAAGTCAAGGACACCGAGGA |
| | | AGAGGACTTCCACGTGGACCAGGTGACCACCGTGAAGGTG |
| | | CCTATGATGAAGCGTTTAGGCATGTTTAACATCCAGCACTG |
| | | TAAGAAGCTGTCCAGCTGGGTGCTGCTGATGAAATACCTGG |
| | | GCAATGCCACCGCCATCTTCTTCCTGCCTGATGAGGGGAAA |
| | | CTACAGCACCTGGAAAATGAACTCACCCACGATATCATCAC |
| | | CAAGTTCCTGGAAAATGAAGACAGAAG |
| 95 | AAT exon 6-7 | GTCTGCCAGCTTACATTTACCCAAACTGTCCATTACTGGAAC |
| | | CTATGATCTGAAGAGCGTCCTGGGTCAACTGGGCATCACTA |
| | | AGGTCTTCAGCAATGGGGCTGACCTCTCCGGGGTCACAGAG |
| | | GAGGCACCCCTGAAGCTCTCCAAGGCCGTGCATAAGGCTGT |
| | | GCTGACCATCGACGAGAAAGGGACTGAAGCTGCTGGGGCC |
| | | ATGTTTTTAGAGGCCATACCCATGTCTATCCCCCCCGAGGTC |
| | | AAGTTCAACAAACCCTTTGTCTTCTTAATGATTGAACAAAA |
| | | TACCAAGTCTCCCCTCTTCATGGGAAAAGTGGTGAATCCCA |
| | | CCCAAAAA |
| 96 | AAT amino | MPSSVSWGILLLAGLCCLVPVSLAEDPQGDAAQKTDTSHHDQ |
| | acid sequence | DHPTFNKI |
| | | TPNLAEFAFSLYRQLAHQSNSTNIFFSPVSIATAFAMLSLGTKA |
| | | DTHDEI |
| | | LEGLNFNLTEIPEAQIHEGFQELLRTLNQPDSQLQLTTGNGLFL |
| | | SEGLKL |
| | | VDKFLEDVKKLYHSEAFTVNFGDTEEAKKQINDYVEKGTQGK |
| | | IVDLVKEL |
| | | DRDTVFALVNYIFFKGKWERPFEVKDTEEEDFHVDQVTTVKV |
| | | PMMKRLGM |
| | | FNIQHCKKLSSWVLLMKYLGNATAIFFLPDEGKLQHLENELTH |
| | | DIITKFL |
| | | ENEDRRSASLHLPKLSITGTYDLKSVLGQLGITKVFSNGADLSG |
| | | VTEEAP |
| | | LKLSKAVHKAVLTIDEKGTEAAGAMFLEAIPMSIPPEVKFNKP |
| | | FVFLMIE |
| | | QNTKSPLFMGKVVNPTQK |

What is claimed is:

1. A method of targeted integration of an exogenous polynucleotide sequence into a gene locus of a cell, the method comprising introducing into the cell:
a. a site-specific nuclease system capable of generating a double-strand break within the gene locus;
b. a recombinant vector comprising a donor polynucleotide, wherein the donor polynucleotide comprises:
i. the exogenous polynucleotide sequence which encodes a protein, wherein the exogenous polynucleotide sequence comprises at least one heterologous intron sequence or a portion thereof; and
ii. 5′ and 3′ homology arms flanking the exogenous polynucleotide sequence, wherein each homology arm is homologous to a portion of the gene locus;
whereupon generation of the double-strand break within the gene locus by the site-specific nuclease system, the nucleic acid sequence of the donor polynucleotide is integrated into the gene locus by homology directed repair (HDR), resulting in exogenous production of the protein from the gene locus of the cell.

2. The method of claim 1, wherein the exogenous polynucleotide sequence comprises 2, 3, 4, 5, or more heterologous intron sequences or portions thereof.

3. The method of claim 1, wherein the site-specific nuclease system comprises a CRISPR nuclease and a single guide RNA (sgRNA) capable of hybridizing to the gene locus.

4.-10. (canceled)

11. The method of claim 1, wherein the vector is selected from the group consisting of viral vectors, plasmids, and ssDNAs.

12. The method of claim 11, wherein the vector is an adeno-associated viral (AAV) vector.

13. (canceled)

14. The method of claim 12, wherein the AAV vector is an AAV6 vector.

15. The method of claim 1, wherein exogenous production of protein from the gene locus of the cell is regulated by the native promoter sequence of the gene locus.

16. The method of claim 1, wherein the cell is a primary cell.

17.-19. (canceled)

20. The method of claim 16, wherein the primary cell is selected from the group consisting of a primary stem cell, primary progenitor cell, and primary somatic cell.

21. The method of claim 16, wherein the stem cell selected from the group consisting of an embryonic stem cell, induced pluripotent stem cell, hematopoietic stem cell, mesenchymal stem cell, neural stem cell, and organ stem cell.

22.-25. (canceled)

26. The method of claim 1, wherein the gene locus of the cell comprises one or more mutations associated with a disease or encodes an aberrant protein.

27. The method of claim 1, wherein integration of the donor polynucleotide sequence corrects a mutation in the cell that is associated with a disease.

28. The method of claim 1, wherein integration of the donor polynucleotide sequence replaces a mutant allele in the cell with a wild-type allele.

29. The method of claim 26, wherein the disease is selected from the group consisting of a hemoglobinopathy, a viral infection, X-linked severe combined immune deficiency, Fanconi anemia, hemophilia, neoplasia, cancer, alpha-1 antitrypsin deficiency, amyotrophic lateral sclerosis, Alzheimer's disease, Parkinson's disease, cystic fibrosis, blood diseases and disorders, inflammation, immune system diseases or disorders, metabolic diseases, liver diseases and disorders, kidney diseases and disorders, muscular diseases and disorders, bone or cartilage diseases and disorders, neurological and neuronal diseases and disorders, cardiovascular diseases and disorders, pulmonary diseases and disorders, and lysosomal storage disorders.

30. The method of claim 1, wherein the gene locus of the cell is a Hemoglobin Subunit gene locus.

31. The method of claim 30, wherein the Hemoglobin Subunit gene is selected from the group consisting of the Hemoglobin Subunit Beta (HBB) gene, the Hemoglobin Subunit Alpha 1 (HBA1) gene, and the Hemoglobin Subunit Alpha 2 (HBA2) gene.

32. The method of claim 30, wherein the Hemoglobin Subunit gene locus comprises one or more genetic mutations associated with a hemoglobinopathy.

33. (canceled)

34. The method of claim 32, wherein the hemoglobinopathy is sickle cell disease, α-thalassemia, β-thalassemia, or δ-thalassemia.

35. The method of claim 30, wherein the at least one heterologous intron sequence or a portion thereof is derived from an intron sequence of a Hemoglobin Subunit gene selected from the group consisting of Hemoglobin Subunit Alpha 1 (HBA1) gene, Hemoglobin Subunit Beta (HBB), Hemoglobin Subunit Delta (HBD), and Hemoglobin Subunit Gamma 2 (HBG2).

36. The method of claim 1, wherein the exogenous polynucleotide sequence encodes beta globin protein.

37. The method of claim 1, wherein the exogenous polynucleotide sequence encodes alpha-1 antitrypsin protein.

38. The method of claim 1, wherein the gene locus of the cell is CCR5.

39. The method of claim 1, wherein the method is performed ex vivo.

40. A composition comprising a population of primary hematopoietic stem and progenitor cells (HSPCs) isolated from a subject, wherein one or more primary HSPCs of the population comprise:
(a) a site-specific nuclease system capable of generating a double-strand break within a gene locus of the HSPC; and
(b) a recombinant vector comprising a donor polynucleotide, wherein the donor polynucleotide comprises:
i. an exogenous polynucleotide sequence which encodes a protein, wherein the exogenous polynucleotide sequence comprises at least one heterologous intron sequence or a portion thereof; and
ii. 5′ and 3′ homology arms flanking the exogenous polynucleotide sequence, wherein each homology arm is homologous to a portion of the gene locus;
whereupon generation of the double-strand break within the gene locus by the site-specific nuclease system, the nucleic acid sequence of the donor polynucleotide is integrated into the gene locus by homology directed repair (HDR), resulting in exogenous production of the protein from the gene locus of the cell.

41.-64. (canceled)

65. A Hemoglobin Subunit Beta (HBB) donor polynucleotide comprising, in a 5′ to 3′ orientation:
a. a first HBB homology region comprising a nucleic acid sequence having at least 95% sequence identity to a first target region of the HBB gene;
b. a diverged HBB exon 1 region comprising a nucleic acid sequence having less than 95% sequence identity to exon 1 of the HBB gene, and which encodes an amino acid sequence encoded by exon 1 of the HBB gene;
c. a heterologous globin intron 1 region comprising a nucleic acid sequence having at least 95% sequence identity to intron 1, or a portion thereof, of a Hemoglobin Subunit gene;
d. a diverged HBB exon 2 region comprising a nucleic acid sequence having less than 95% sequence identity to exon 2 of the HBB gene, and which encodes an amino acid sequence encoded by exon 2 of the HBB gene;
e. a heterologous globin intron 2 region comprising a nucleic acid sequence having at least 95% sequence identity to intron 2, or a portion thereof, of a Hemoglobin Subunit gene;
f. a diverged HBB exon 3 region comprising a nucleic acid sequence having less than 95% sequence identity to exon 3 of the HBB gene, and which encodes an amino acid sequence encoded by exon 3 of the HBB gene; and
g. a second HBB homology region comprising a nucleic acid sequence having at least 95% sequence identity to a second target region of the HBB gene, wherein the second target region is positioned 3′ to the first target region in the HBB gene;
wherein homology directed repair (HDR)-mediated integration of the donor polynucleotide sequence into an HBB locus results in exogenous expression of beta globin protein from the HBB locus.

66. The HBB donor polynucleotide of claim 65, further comprising a polyadenylation signal sequence positioned between the diverged HBB exon 3 and the second HBB homology region.

67. The HBB donor polynucleotide of claim 66, wherein the polyadenylation signal sequence is selected from the group consisting of a polyadenylation signal sequence from bovine growth hormone (bGH), human growth hormone (hGH), rabbit beta globin (RbGlob), a synthetic poly A sequence based on rabbit beta globin poly A (SynthRbGlob) and Simian Virus 40 (SV40).

68. (canceled)

69. (canceled)

70. The HBB donor polynucleotide of claim 65, wherein the diverged HBB exon 1 region comprises a nucleic acid sequence having between 60% and 90% sequence identity to exon 1 of the HBB gene.

71. (canceled)

72. The HBB donor polynucleotide of claim 65, wherein the diverged HBB exon 2 region comprises a nucleic acid sequence having between 57% and 90% sequence identity to exon 2 of the HBB gene.

73. (canceled)

74. The HBB donor polynucleotide of claim 65, wherein the diverged HBB exon 3 region comprises a nucleic acid sequence having between 62% and 90% sequence identity to exon 3 of the HBB gene.

75. (canceled)

76. The HBB donor polynucleotide of claim 65, wherein the heterologous globin intron 1 region comprises a nucleic acid sequence having at least 95% sequence identity to intron 1, or a portion thereof, of a Hemoglobin Subunit gene selected from the group consisting of Hemoglobin Subunit Alpha 1 (HBA1), Hemoglobin Subunit Beta (HBB), Hemoglobin Subunit Delta (HBD), and Hemoglobin Subunit Gamma 2 (HBG2).

77. (canceled)

78. (canceled)

79. The HBB donor polynucleotide of claim 65, wherein the heterologous globin intron 2 region comprises a nucleic acid sequence having at least 95% sequence identity to intron 2, or a portion thereof, of a Hemoglobin Subunit gene selected from the group consisting of Hemoglobin Subunit Alpha 1 (HBA1), Hemoglobin Subunit Beta (HBB), Hemoglobin Subunit Delta (HBD), and Hemoglobin Subunit Gamma 2 (HBG2).

80. (canceled)

81. (canceled)

82. The HBB donor polynucleotide of claim 65, wherein the heterologous globin intron 2 region comprises a truncated intron 2 of a Hemoglobin Subunit gene, wherein the truncation comprises deletion of nucleotides 21-437 and 513-834 of the intron.

83. The HBB donor polynucleotide of claim 82, wherein the truncated intron 2 comprises a truncated HBG2 intron 2 nucleic acid sequence.

84.-94. (canceled)

95. A method of expressing exogenous beta globin protein in a cell, the method comprising introducing into the cell:
a. a site-specific nuclease system capable of generating a double-strand break within the HBB gene; and
b. a recombinant vector comprising the HBB donor polynucleotide of claim 65;
whereupon generation of the double-strand break within the HBB gene by the site-specific nuclease system, the nucleic acid sequence of the donor polynucleotide is integrated into the HBB locus by homology directed repair (HDR), resulting in exogenous production of beta globin protein from the HBB locus of the cell.

96.-118. (canceled)

119. A composition comprising a population of primary hematopoietic stem and progenitor cells (HSPCs) isolated from a subject, wherein one or more primary HSPCs of the population comprise:
a. a site-specific nuclease system capable of generating a double-strand break within the HBB gene; and
b. a recombinant vector comprising the HBB donor polynucleotide of claim 65.

120.-137. (canceled)

138. A pharmaceutical composition comprising an isolated population of primary hematopoietic stem and progenitor cells (HSPCs) derived from an individual subject having a hemoglobinopathy resulting from one or mutations in the HBB gene, wherein the HSPC population comprises:
a. a first plurality of primary HSPCs comprising the one or more mutations in the HBB gene; and
b. a second plurality of primary HSPCs comprising a heterologous polynucleotide integrated into the HBB locus, wherein the heterologous polynucleotide comprises the nucleic acid sequence of the HBB donor polynucleotide of claim 65.

139.-142. (canceled)

143. A method for preventing or treating a hemoglobinopathy resulting from one or mutations in the HBB gene in a subject in need thereof, the method comprising administering to the subject the pharmaceutical composition of claim 138.

144.-149. (canceled)

150. An isolated primary HSPC comprising a heterologous polynucleotide integrated into the HBB locus, wherein the heterologous polynucleotide comprises the nucleic acid sequence of the HBB donor polynucleotide of claim 65.

151. An alpha-1 antitrypsin (AAT) donor polynucleotide comprising, in a 5′ to 3′ orientation:
a. a first Hemoglobin Subunit Alpha 1 (HBA1) homology region comprising a nucleic acid sequence having at least 95% sequence identity to a first target region of the HBA1 gene;
b. an exon 1 region comprising a nucleic acid sequence having at least 95% sequence identity to exon 4 of the AAT gene, and which encodes an amino acid sequence encoded by exon 4 of the AAT gene;
c. a heterologous globin intron 1 region comprising a nucleic acid sequence having at least 95% sequence identity to intron 1, or a portion thereof, of a Hemoglobin Subunit gene;
d. an exon 2 region comprising a nucleic acid sequence having at least 95% sequence identity to exon 5 of the AAT gene, and which encodes an amino acid sequence encoded by exon 5 of the AAT gene;
e. a heterologous globin intron 2 region comprising a nucleic acid sequence having at least 95% sequence identity to intron 2, or a portion thereof, of a Hemoglobin Subunit gene;
f. an exon 3 region comprising a nucleic acid sequence having at least 95% sequence identity to exon 6-7 of the AAT gene, and which encodes an amino acid sequence encoded by exon 6-7 of the AAT gene; and
g. a second HBA1 homology region comprising a nucleic acid sequence having at least 95% sequence identity to a second target region of the HBA1 gene, wherein the second target region is positioned 3′ to the first target region in the HBA1 gene;
wherein homology directed repair (HDR)-mediated integration of the ATT donor polynucleotide sequence into an HBA1 locus results in exogenous expression of alpha-1 antitrypsin protein from the HBA1 locus.

152.-178. (canceled)


SEQUENCE LISTING (97 sequences)

SEQ ID NO: 1 - DNA, 1421 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaaggttggtatcaaggttacaagacaggtttaaggagaccaatagaaactgggcatgtggagacagagaagactcttgggtttctgataggcactgactctctctgcctattggtctattttcccacccttagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctgctcgggaatgtcctcgtgtgcgtcctcgctcaccatttcgggaaggagtttacaccccccgtccaagccgcttaccaaaaggtcgtcgccggagtcgccaacgctctcgctcataaataccat

SEQ ID NO: 2 - DNA, 1449 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaaggtaggctctggtgaccaggacaagggagggaaggaaggaccctgtgcctggcaaaagtccaggtcgcttctcaggatttgtggcaccttctgactgtcaaactgttcttgtcaatctcacagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgcgcgcgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagcttggtgtgtagttatctggaggccaggctggagctctcagctcactatgggttcatctttattgtctcctttcatctcaacagctgctcgggaatgtcctcgtgtgcgtcctcgctcaccatttcgggaaggagtttacaccccccgtccaagccgcttaccaaaaggtcgtcgccggagtcgccaacgctctcgctcataaataccat

SEQ ID NO: 3 - DNA, 1467 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaaggttggtatcaaggttataagagaggctcaaggaggcaaatggaaactgggcatgtgtagacagagaagactcttgggtttctgataggcactgactctctgtcccttgggctgttttcctaccctcagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgcttcacttttctctttttactttctaatcttacattttggttcttttacctacctgctcttctcccacatttttgtcattttactatattttatcatttaatgcttctaaaattttgttaattttttatttaaatattctgcattttttccttcctcacaatcttgctattttaaattatttaatatcctgtctttctctcccaaccccctcccttcatttttccttctctaacaacaactcaaattatgcataccagctctcacctgctaattctgcacttagaataatccttttgtctctccacatgggtatgggagaggctccaactcaaagatgagaggcatagaatactgttttagaggctataaatcattttacaataaggaataattggaattttataaattctgtagtaaatggaatggaaaggaaagtgaatatttgattatgaaagactaggcagttacactggaggtggggcagaagtcgttgctaggagacagcccatcatcacactgattaatcaattaatttgtatctattaatctgtttatagtaattaatttgtatatgctatatacacatacaaaattaaaactaatttggaattaatttgtatatagtattatacagcatatatagcatatatgtacatatatagactacatgctagttaagtacatagaggatgtgtgtgtatagatatatgttatatgtatgcattcatatatgtacttatttatgctgatgggaataacctggggatcagttttgtctaagatttgggcagaaaaaaatgggtgttggctcagtttctcagaagccagtctttatttctctgttaaccatatgcatgtatctgcctacctcttctccgcagctgctcgggaatgtcctcgtgtgcgtcctcgctcaccatttcgggaaggagtttacaccccccgtccaagccgcttaccaaaaggtcgtcgccggagtcgccaacgctctcgctcataaataccat

SEQ ID NO: 4 - DNA, 1461 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaaggtaggctctggtgaccaggacaagggagggaaggaaggaccctgtgcctggcaaaagtccaggtcgcttctcaggatttgtggcaccttctgactgtcaaactgttcttgtcaatctcacagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgcttcacttttctctttttactttctaatcttacattttggttcttttacctacctgctcttctcccacatttttgtcattttactatattttatcatttaatgcttctaaaattttgttaattttttatttaaatattctgcattttttccttcctcacaatcttgctattttaaattatttaatatcctgtctttctctcccaaccccctcccttcatttttccttctctaacaacaactcaaattatgcataccagctctcacctgctaattctgcacttagaataatccttttgtctctccacatgggtatgggagaggctccaactcaaagatgagaggcatagaatactgttttagaggctataaatcattttacaataaggaataattggaattttataaattctgtagtaaatggaatggaaaggaaagtgaatatttgattatgaaagactaggcagttacactggaggtggggcagaagtcgttgctaggagacagcccatcatcacactgattaatcaattaatttgtatctattaatctgtttatagtaattaatttgtatatgctatatacacatacaaaattaaaactaatttggaattaatttgtatatagtattatacagcatatatagcatatatgtacatatatagactacatgctagttaagtacatagaggatgtgtgtgtatagatatatgttatatgtatgcattcatatatgtacttatttatgctgatgggaataacctggggatcagttttgtctaagatttgggcagaaaaaaatgggtgttggctcagtttctcagaagccagtctttatttctctgttaaccatatgcatgtatctgcctacctcttctccgcagctgctcgggaatgtcctcgtgtgcgtcctcgctcaccatttcgggaaggagtttacaccccccgtccaagccgcttaccaaaaggtcgtcgccggagtcgccaacgctctcgctcataaataccat

SEQ ID NO: 5 - DNA, 1465 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaaggttggtatcaatgttataagagaggctcatggaggtaaatggaagctgggcatgtgtagacagagaagactctggaggttctgatagtcattgattctctctgtcccttgggctgttttcctaccctcagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgcttcacttttctctgtttactgtctaatcttacattttagtttttacctacctgctcttcccccacatttttgtcattttactatattttatcatttaatgcttctaaaattttgttatttttttatttaaatattctgcattttttccttcctcacaatcttgctattttaaattatttaatatcctgtcctctcctccccaaccccttcccttcgttttcttctctaaccacaactcaaattatgcatgccagctctcacgtgctaattctgcacttagaataattctttgtctctccacatgggtatgagagaggctccagctcaaagacgagaggcatagaatactgttttagaggctataaattattttacaataaggaataattggaattttataaatttggtagtaaatgggatggaaaggaaagtgaatatttgattatgaaagactagaaagttacactggaggtggggcagaagtcgttgctaggagacagcccatcatcacactgattaatgaattaatttgtatctattaatctgtttagagtaattaatttgtatatgctatatacacatacaaaattaaaactaatttggaattaatttgtatatagcattatacagcatatatagcatatatgtacatatatagactatatgctagttaagtacacagaggatgtgtgtgtatagatatatgttatatgcatgcattcatatatgtacttatttatgctgatgggaataacctggggatcagttttgtctaagatttgcgcagaaaaaaatgggtgttggcccagtttctcagaagccaatctttatttctctgttaaccatatgcatatatctgcctaccttttctctgcagctgctcgggaatgtcctcgtgtgcgtcctcgctcaccatttcgggaaggagtttacaccccccgtccaagccgcttaccaaaaggtcgtcgccggagtcgccaacgctctcgctcataaataccat

SEQ ID NO: 6 - DNA, 1421 bp, Homo sapiens
atggtgcatctgactcctgaggagaagtctgccgttactgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggcccacaagtatcac

SEQ ID NO: 7 - DNA, 441 bp, Homo sapiens
atggtgcatctgactcctgaggagaagtctgccgttactgccctgtggggcaaggtgaacgtggatgaagttggtggtgaggccctgggcaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcaggctcctgggcaacgtgctggtctgtgtgctggcccatcactttggcaaagaattcaccccaccagtgcaggctgcctatcagaaagtggtggctggtgtggctaatgccctggcccacaagtatcac

SEQ ID NO: 8 - DNA, 441 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagactgctcgggaatgtcctcgtgtgcgtcctcgctcaccatttcgggaaggagtttacaccccccgtccaagccgcttaccaaaaggtcgtcgccggagtcgccaacgctctcgctcataaataccat

SEQ ID NO: 9 - DNA, 130 bp, Homo sapiens
gttggtatcaaggttacaagacaggtttaaggagaccaatagaaactgggcatgtggagacagagaagactcttgggtttctgataggcactgactctctctgcctattggtctattttcccacccttag

SEQ ID NO: 10 - DNA, 850 bp, Homo sapiens
gtgagtctatgggacgcttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 11 - DNA, 122 bp, Homo sapiens
gtaggctctggtgaccaggacaagggagggaaggaaggaccctgtgcctggcaaaagtccaggtcgcttctcaggatttgtggcaccttctgactgtcaaactgttcttgtcaatctcacag

SEQ ID NO: 12 - DNA, 886 bp, Homo sapiens
gtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgcgcgcgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagcttggtgtgtagttatctggaggccaggctggagctctcagctcactatgggttcatctttattgtctcctttcatctcaacag

SEQ ID NO: 13 - DNA, 128 bp, Homo sapiens
gttggtatcaaggttataagagaggctcaaggaggcaaatggaaactgggcatgtgtagacagagaagactcttgggtttctgataggcactgactctctgtcccttgggctgttttcctaccctcag

SEQ ID NO: 14 - DNA, 898 bp, Homo sapiens
gtgagtccaggagatgcttcacttttctctttttactttctaatcttacattttggttcttttacctacctgctcttctcccacatttttgtcattttactatattttatcatttaatgcttctaaaattttgttaattttttatttaaatattctgcattttttccttcctcacaatcttgctattttaaattatttaatatcctgtctttctctcccaaccccctcccttcatttttccttctctaacaacaactcaaattatgcataccagctctcacctgctaattctgcacttagaataatccttttgtctctccacatgggtatgggagaggctccaactcaaagatgagaggcatagaatactgttttagaggctataaatcattttacaataaggaataattggaattttataaattctgtagtaaatggaatggaaaggaaagtgaatatttgattatgaaagactaggcagttacactggaggtggggcagaagtcgttgctaggagacagcccatcatcacactgattaatcaattaatttgtatctattaatctgtttatagtaattaatttgtatatgctatatacacatacaaaattaaaactaatttggaattaatttgtatatagtattatacagcatatatagcatatatgtacatatatagactacatgctagttaagtacatagaggatgtgtgtgtatagatatatgttatatgtatgcattcatatatgtacttatttatgctgatgggaataacctggggatcagttttgtctaagatttgggcagaaaaaaatgggtgttggctcagtttctcagaagccagtctttatttctctgttaaccatatgcatgtatctgcctacctcttctccgcag

SEQ ID NO: 15 - DNA, 130 bp, Artificial Sequence
  Description of Artificial Sequence Synthetic polynucleotide
gttggtatcaatgttataagagaggctcatggaggtaaatggaagctgggcatgtgtagacagagaagactctggaggttctgatagtcattgattctctctgtcccttgggctgttttcctaccctcag

SEQ ID NO: 16 - DNA, 894 bp, Artificial Sequence
  Description of Artificial Sequence Synthetic polynucleotide
gtgagtccaggagatgcttcacttttctctgtttactgtctaatcttacattttagtttttacctacctgctcttcccccacatttttgtcattttactatattttatcatttaatgcttctaaaattttgttatttttttatttaaatattctgcattttttccttcctcacaatcttgctattttaaattatttaatatcctgtcctctcctccccaaccccttcccttcgttttcttctctaaccacaactcaaattatgcatgccagctctcacgtgctaattctgcacttagaataattctttgtctctccacatgggtatgagagaggctccagctcaaagacgagaggcatagaatactgttttagaggctataaattattttacaataaggaataattggaattttataaatttggtagtaaatgggatggaaaggaaagtgaatatttgattatgaaagactagaaagttacactggaggtggggcagaagtcgttgctaggagacagcccatcatcacactgattaatgaattaatttgtatctattaatctgtttagagtaattaatttgtatatgctatatacacatacaaaattaaaactaatttggaattaatttgtatatagcattatacagcatatatagcatatatgtacatatatagactatatgctagttaagtacacagaggatgtgtgtgtatagatatatgttatatgcatgcattcatatatgtacttatttatgctgatgggaataacctggggatcagttttgtctaagatttgcgcagaaaaaaatgggtgttggcccagtttctcagaagccaatctttatttctctgttaaccatatgcatatatctgcctaccttttctctgcag

SEQ ID NO: 17 - DNA, 495 bp, Homo sapiens
gtcctgtaagtattttgcatattctggagacgcaggaagagatccatctacatatcccaaagctgaattatggtagacaaaactcttccacttttagtgcatcaacttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacacc

SEQ ID NO: 18 - DNA, 426 bp, Homo sapiens
gctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcatatgccttattcatccctcagaaaaggattcaagtagaggcttgatttggaggttaaagttttgctatgctgtattttacattacttattgttttagctgtcctcatgaa

SEQ ID NO: 19 - DNA, 495 bp, Homo sapiens; identical to SEQ ID NO: 17

SEQ ID NO: 20 - DNA, 426 bp, Homo sapiens
ctgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacgct

SEQ ID NO: 21 - DNA, 400 bp, Artificial Sequence
  Description of Artificial Sequence Synthetic polynucleotide
cagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattcaccccaccagtgcaggctgcctatcagaaagtggtggctggtgtggctaatgccctggctcataaataccat

SEQ ID NO: 22 - DNA, 397 bp, Homo sapiens
gctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcatatgccttattcatccctcagaaaaggattcaagtagaggcttgatttggaggttaaagttttgctatgctgtattttaca

SEQ ID NO: 23 - DNA, 946 bp, Homo sapiens
gctccagccggttccagctattgctttgtttacctgtttaaccagtatttacctagcaagtcttccatcagatagcatttggagagctgggggtgtcacagtgaaccacgacctctaggccagtgggagagtcagtcacacaaactgtgagtccatgacttggggcttagccagcacccaccaccccacgcgccaccccacaaccccgggtagaggagtctgaatctggagccgcccccagcccagccccgtgctttttgcgtcctggtgtttattccttcccggtgcctgtcactcaagcacactagtgactatcgccagagggaaagggagctgcaggaagcgaggctggagagcaggaggggctctgcgcagaaattcttttgagttcctatgggccagggcgtccgggtgcgcgcattcctctccgccccaggattgggcgaagcctcccggctcgcactcgctcgcccgtgtgttccccgatcccgctggagtcgatgcgcgtccagcgcgtgccaggccggggcgggggtgcgggctgactttctccctcgctagggacgctccggcgcccgaaaggaaagggtggcgctgcgctccggggtgcacgagccgacagcgcccgaccccaacgggccggccccgccagcgccgctaccgccctgcccccgggcgagcgggatgggcgggagtggagtggcgggtggagggtggagacgtcctggcccccgccccgcgtgcacccccaggggaggccgagcccgccgcccggccccgcgcaggccccgcccgggactcccctgcggtccaggccgcgccccgggctccgcgccagccaatgagcgccgcccggccgggcgtgcccccgcgccccaagcataaaccctggcgcgctcgcggcccggcactcttctggtccccacagactcagagagaacccacc

SEQ ID NO: 24 - DNA, 879 bp, Homo sapiens
tggccatgcttcttgccccttgggcctccccccagcccctcctccccttcctgcacccgtacccccgtggtctttgaataaagtctgagtgggcggcagcctgtgtgtgcctgagttttttccctcagcaaacgtgccaggcatgggcgtggacagcagctgggacacacatggctagaacctctctgcagctggatagggtaggaaaaggcaggggcgggaggaggggatggaggagggaaagtggagccaccgcgaagtccagctggaaaaacgctggaccctagagtgctttgaggatgcatttgctctttcccgagttttattcccagacttttcagattcaatgcaggtttgctgaaataatgaatttatccatctttacgtttctgggcactctgtgccaagaactggctggctttctgcctgggacgtcactggtttcccagaggtcctcccacatatgggtggtgggtaggtcagagaagtcccactccagcatggctgcattgatcccccatcgttcccactagtctccgtaaaacctcccagatacaggcacagtctagatgaaatcaggggtgcggggtgcaactgcaggccccaggcaattcaataggggctctactttcacccccaggtcaccccagaatgctcacacaccagacactgacgccctggggctgtcaagatcaggcgtttgtctctgggcccagctcagggcccagctcagcacccactcagctcccctgaggctggggagcctgtcccattgcgactggagaggagagcggggccacagaggcctggctagaaggtcccttctccctggtgtgtgttttctctctgctgagcaggcttgcagtgcctggggtatca

SEQ ID NO: 25 - DNA, 20 bp, Homo sapiens
ggcaagaagcatggccaccg

SEQ ID NO: 26 - DNA, 20 bp, Homo sapiens
agcgagcttagtgatacttg

SEQ ID NO: 27 - DNA, 20 bp, Homo sapiens
cttgccccacagggcagtaa

SEQ ID NO: 28 - DNA, 117 bp, Homo sapiens
gtgaggctccctcccctgctccgacccgggctcctcgcccgcccggacccacaggccaccctcaaccgtcctggccccggacccaaaccccacccctcactctgcttctccccgcag

SEQ ID NO: 29 - DNA, 149 bp, Homo sapiens
gtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacag

SEQ ID NO: 30 - DNA, 710 bp, Artificial Sequence
  Description of Artificial Sequence Synthetic polynucleotide
atggtgcacctgactcctgaggagaagtctgccgtgaccgctctctggggaaaagtcaatgtcgacgaggtgggaggcgaagctctcggaaggtgaggctccctcccctgctccgacccgggctcctcgcccgcccggacccacaggccaccctcaaccgtcctggccccggacccaaaccccacccctcactctgcttctccccgcagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacagctgctcgggaatgtcctcgtgtgcgtcctcgctcaccatttcgggaaggagtttacaccccccgtccaagccgcttaccaaaaggtcgtcgccggagtcgccaacgctctcgctcataaataccattaa

SEQ ID NO: 31 - DNA, 720 bp, Artificial Sequence
  Description of Artificial Sequence Synthetic polynucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 32 - DNA, 208 bp, Artificial Sequence
  Description of Artificial Sequence Synthetic polynucleotide
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 33 - DNA, 638 bp, Artificial Sequence
  Description of Artificial Sequence Synthetic polynucleotide
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaataaaagatctttattttcattagatctgtgtgttggttttttgtgtg

SEQ ID NO: 34 - DNA, 92 bp, Homo sapiens
atggtgcatctgactcctgaggagaagtctgccgttactgccctgtggggcaaggtgaacgtggatgaagttggtggtgaggccctgggcag

SEQ ID NO: 35 - DNA, 92 bp, Artificial Sequence
  Description of Artificial Sequence Synthetic oligonucleotide
atggtccacctcacccccgaagaaaaatccgcagtcaccgctctctggggaaaagtcaatgtcgacgaggtgggaggcgaagctctcggaag

SEQ ID NO: 36 - DNA, 223 bp, Artificial Sequence
  Description of Artificial Sequence Synthetic polynucleotide
actcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttaga

SEQ ID NO: 37 - DNA, 126 bp, Artificial Sequence
  Description of Artificial Sequence Synthetic polynucleotide
ctgctcgggaatgtcctcgtgtgcgtcctcgctcaccatttcgggaaggagtttacaccccccgtccaagccgcttaccaaaaggtcgtcgccggagtcgccaacgctctcgctcataaataccat

SEQ ID NO: 38 - DNA, 19 bp, Artificial Sequence
  Description of Artificial Sequence Synthetic oligonucleotide
gtggaaaaatccgcagtca

SEQ ID NO: 39 - DNA, 1192 bp, Artificial Sequence
  Description of Artificial Sequence Synthetic polynucleotide
aatttagtacaaggggaaaaagtacagggggatgggagaaaggcgatcacgttgggaagctatagagaaagaagagtaaattttagtaaaggaggtttaaacaaacaaaatataaagagaaataggaacttgaatcaaggaaatgattttaaaacgcagtattcttagtggactagaggaaaaaaataatctgagccaagtagaagaccttttcccctcctacccctactttctaagtcacagaggctttttgttcccccagacactcttgcagattagtccaggcagaaacagttagatgtccccagttaacctcctatttgacaccactgattaccccattgatagtcacactttgggttgtaagtgactttttatttatttgtatttttgactgcattaagaggtctctagttttttacctcttgtttcccaaaacctaataagtaactaatgcacagagcacattgatttgtatttattctatttttagacataatttattagcatgcatgagcaaattaagaaaaacaacaacaaatgaatgcatatatatgtatatgtatgtgtgtacatatacacatatatatatatattttttttcttttcttaccagaaggttttaatccaaataaggagaagatatgcttagaactgaggtagagttttcatccattctgtcctgtaagtattttgcatattctggagacgcaggaagagatccatctacatatcccaaagctgaattatggtagacaaaactcttccacttttagtgcatcaatttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacaccatggtgcacctgactcct

SEQ ID NO: 40 - DNA, 1192 bp, Artificial Sequence
  Description of Artificial Sequence Synthetic polynucleotide
ctgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacccttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttattttcttttatttttaattgatacataatcattatacatatttatgggttaaagtgtaatgttttaatatgtgtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatctctgcatataaatatttctgcatataaattgtaactgatgtaagaggtttcatattgctaatagcagctacaatccagctaccattctgcttttattttatggttgggataaggctgg

SEQ ID NO: 41 - DNA, 444 bp, Artificial Sequence
  Description of Artificial Sequence Synthetic polynucleotide
atggtccacctcacccccgtggaaaaatccgcagtcaccgctctctggggaaaagtcaatgtcgacgaggtgggaggcgaagctctcggaagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagactgctcgggaatgtcctcgtgtgcgtcctcgctcaccatttcgggaaggagtttacaccccccgtccaagccgcttaccaaaaggtcgtcgccggagtcgccaacgctctcgctcataaataccattaa

SEQ ID NO: 42 - DNA, 1452 bp, Artificial Sequence
  Description of Artificial Sequence Synthetic polynucleotide
atggtccacctcacccccgtggaaaaatccgcagtcaccgctctctggggaaaagtcaatgtcgacgaggtgggaggcgaagctctcggaaggtaggctctggtgaccaggacaagggagggaaggaaggaccctgtgcctggcaaaagtccaggtcgcttctcaggatttgtggcaccttctgactgtcaaactgttcttgtcaatctcacagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgcgcgcgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagcttggtgtgtagttatctggaggccaggctggagctctcagctcactatgggttcatctttattgtctcctttcatctcaacagctgctcgggaatgtcctcgtgtgcgtcctcgctcaccatttcgggaaggagtttacaccccccgtccaagccgcttaccaaaaggtcgtcgccggagtcgccaacgctctcgctcataaataccattaa

SEQ ID NO: 43 - DNA, 1470 bp, Artificial Sequence
  Description of Artificial Sequence Synthetic polynucleotide
atggtccacctcacccccgtggaaaaatccgcagtcaccgctctctggggaaaagtcaatgtcgacgaggtgggaggcgaagctctcggaaggttggtatcaaggttataagagaggctcaaggaggcaaatggaaactgggcatgtgtagacagagaagactcttgggtttctgataggcactgactctctgtcccttgggctgttttcctaccctcagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgcttcacttttctctttttactttctaatcttacattttggttcttttacctacctgctcttctcccacatttttgtcattttactatattttatcatttaatgcttctaaaattttgttaattttttatttaaatattctgcattttttccttcctcacaatcttgctattttaaattatttaatatcctgtctttctctcccaaccccctcccttcatttttccttctctaacaacaactcaaattatgcataccagctctcacctgctaattctgcacttagaataatccttttgtctctccacatgggtatgggagaggctccaactcaaagatgagaggcatagaatactgttttagaggctataaatcattttacaataaggaataattggaattttataaattctgtagtaaatggaatggaaaggaaagtgaatatttgattatgaaagactaggcagttacactggaggtggggcagaagtcgttgctaggagacagcccatcatcacactgattaatcaattaatttgtatctattaatctgtttatagtaattaatttgtatatgctatatacacatacaaaattaaaactaatttggaattaatttgtatatagtattatacagcatatatagcatatatgtacatatatagactacatgctagttaagtacatagaggatgtgtgtgtatagatatatgttatatgtatgcattcatatatgtacttatttatgctgatgggaataacctggggatcagttttgtctaagatttgggcagaaaaaaatgggtgttggctcagtttctcagaagccagtctttatttctctgttaaccatatgcatgtatctgcctacctcttctccgcagctgctcgggaatgtcctcgtgtgcgtcctcgctcaccatttcgggaaggagtttacaccccccgtccaagccgcttaccaaaaggtcgtcgccggagtcgccaacgctctcgctcataaataccattaa

SEQ ID NO: 44 - DNA, 1468 bp, Artificial Sequence
  Description of Artificial Sequence Synthetic polynucleotide
atggtccacctcacccccgtggaaaaatccgcagtcaccgctctctggggaaaagtcaatgtcgacgaggtgggaggcgaagctctcggaaggttggtatcaatgttataagagaggctcatggaggtaaatggaagctgggcatgtgtagacagagaagactctggaggttctgatagtcattgattctctctgtcccttgggctgttttcctaccctcagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgcttcacttttctctgtttactgtctaatcttacattttagtttttacctacctgctcttcccccacatttttgtcattttactatattttatcatttaatgcttctaaaattttgttatttttttatttaaatattctgcattttttccttcctcacaatcttgctattttaaattatttaatatcctgtcctctcctccccaaccccttcccttcgttttcttctctaaccacaactcaaattatgcatgccagctctcacgtgctaattctgcacttagaataattctttgtctctccacatgggtatgagagaggctccagctcaaagacgagaggcatagaatactgttttagaggctataaattattttacaataaggaataattggaattttataaatttggtagtaaatgggatggaaaggaaagtgaatatttgattatgaaagactagaaagttacactggaggtggggcagaagtcgttgctaggagacagcccatcatcacactgattaatgaattaatttgtatctattaatctgtttagagtaattaatttgtatatgctatatacacatacaaaattaaaactaatttggaattaatttgtatatagcattatacagcatatatagcatatatgtacatatatagactatatgctagttaagtacacagaggatgtgtgtgtatagatatatgttatatgcatgcattcatatatgtacttatttatgctgatgggaataacctggggatcagttttgtctaagatttgcgcagaaaaaaatgggtgttggcccagtttctcagaagccaatctttatttctctgttaaccatatgcatatatctgcctaccttttctctgcagctgctcgggaatgtcctcgtgtgcgtcctcgctcaccatttcgggaaggagtttacaccccccgtccaagccgcttaccaaaaggtcgtcgccggagtcgccaacgctctcgctcataaataccattaa

SEQ ID NO: 45 - DNA, 124 bp, Homo sapiens
gttctcttccagtttgcaggtgttcctgtgaccctgacaccctccttctgcacatggggactgggcttggccttgagagaaagccttctgtttaataaagtacattttcttcagtaatcaaaaa

SEQ ID NO: 46 - DNA, 89 bp, Homo sapiens
gctcactgcccatgatgcagagctttcaaggataggctttattctgcaagcaatcaaataataaatctattctgctaagagatcacaca

SEQ ID NO: 47 - DNA, 90 bp, Homo sapiens
gctcactgcccatgattcagagctttcaaggataggctttattctgcaagcaatacaaataataaatctattctgctgagagatcacaca

SEQ ID NO: 48 - DNA, 126 bp, Homo sapiens
gatcctggactgtttcctgataaccataagaagaccctatttccctagattctattttctgaacttgggaacacaatgcctacttcaagggtatggcttctgcctaataaagaatgttcagctcaa

SEQ ID NO: 49 - DNA, 103 bp, Homo sapiens
gcgccgcctccgggacccccaggacaggctgcggcccctcccccgtcctggaggttccccagccccacttaccgcgtaatgcgccaataaaccaatgaacgaa

SEQ ID NO: 50 - DNA, 110 bp, Homo sapiens
gctggagcctcggtagccgttcctcctgcccgctgggcctcccaacgggccctcctcccctccttgcaccggcccttcctggtctttgaataaagtctgagtgggcagca

SEQ ID NO: 51 - DNA, 111 bp, Homo sapiens
gctggagcctcggtggccatgcttcttgccccttgggcctccccccagcccctcctccccttcctgcacccgtacccccgtggtctttgaataaagtctgagtgggcggca

SEQ ID NO: 52 - DNA, 477 bp, Artificial Sequence
  Description of Artificial Sequence Synthetic polynucleotide
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 53 - DNA, 56 bp, Artificial Sequence
  Description of Artificial Sequence Synthetic oligonucleotide
aataaaggaaatttattttcattgcaatagtgtgttggaattttttgtgtctctca

SEQ ID NO: 54 - DNA, 49 bp, Artificial Sequence
  Description of Artificial Sequence Synthetic oligonucleotide
aataaaagatctttattttcattagatctgtgtgttggttttttgtgtg

SEQ ID NO: 55 - DNA, 122 bp, Artificial Sequence
  Description of Artificial Sequence Synthetic polynucleotide
aacttgtttattgcagcttataatggttacaaataaagcaatagcatcacaaatttcacaaataaagcatttttttcactgcattctagttgtggtttgtccaaactcatcaatgtatctta

SEQ ID NO: 56 - DNA, 1449 bp, Artificial Sequence; identical to SEQ ID NO: 2
  Description of Artificial Sequence Synthetic polynucleotide

SEQ ID NO: 57 - DNA, 441 bp, Artificial Sequence
  Description of Artificial Sequence Synthetic polynucleotide
atggtgcacctgactcctgaggagaagtctgccgtgaccgctctctggggaaaagtcaatgtcgacgaggtgggaggcgaagctctcggaagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagactgctcgggaatgtcctcgtgtgcgtcctcgctcaccatttcgggaaggagtttacaccccccgtccaagccgcttaccaaaaggtcgtcgccggagtcgccaacgctctcgctcataaataccat

SEQ ID NO: 58 - DNA, 1315 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgcgcgcgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagcttggtgtgtagttatctggaggccaggctggagctctcagctcactatgggttcatctttattgtctcctttcatctcaacagctgctcgggaatgtcctcgtgtgcgtcctcgctcaccatttcgggaaggagtttacaccccccgtccaagccgcttaccaaaaggtcgtcgccggagtcgccaacgctctcgctcataaataccat

SEQ ID NO: 59 - DNA, 563 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaaggtaggctctggtgaccaggacaagggagggaaggaaggaccctgtgcctggcaaaagtccaggtcgcttctcaggatttgtggcaccttctgactgtcaaactgttcttgtcaatctcacagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagactgctcgggaatgtcctcgtgtgcgtcctcgctcaccatttcgggaaggagtttacaccccccgtccaagccgcttaccaaaaggtcgtcgccggagtcgccaacgctctcgctcataaataccat

SEQ ID NO: 60 - DNA, 1390 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaaggtaggctctggtgaccaggattctcaggatttgtggcaccttctgactgtcaaactgttcttgtcaatctcacagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgcgcgcgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagcttggtgtgtagttatctggaggccaggctggagctctcagctcactatgggttcatctttattgtctcctttcatctcaacagctgctcgggaatgtcctcgtgtgcgtcctcgctcaccatttcgggaaggagtttacaccccccgtccaagccgcttaccaaaaggtcgtcgccggagtcgccaacgctctcgctcataaataccat

SEQ ID NO: 61 - DNA, 921 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaaggtaggctctggtgaccaggacaagggagggaaggaaggaccctgtgcctggcaaaagtccaggtcgcttctcaggatttgtggcaccttctgactgtcaaactgttcttgtcaatctcacagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggagctggacacatataaaatgctgctaatgcttcattacaaacttatatcctttaattccagatgggggcaaagtatagctctcagctcactatgggttcatctttattgtctcctttcatctcaacagctgctcgggaatgtcctcgtgtgcgtcctcgctcaccatttcgggaaggagtttacaccccccgtccaagccgcttaccaaaaggtcgtcgccggagtcgccaacgctctcgctcataaataccat

SEQ ID NO: 62 - DNA, 710 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaaggtaggctctggtgaccaggacaagggagggaaggaaggaccctgtgcctggcaaaagtccaggtcgcttctcaggatttgtggcaccttctgactgtcaaactgttcttgtcaatctcacagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgtttcgctggacacatataaaatgctgctaatgcttcattacaaacttatatcctttaattccagatgggggcaaagtatagctctcagctcactatgggttcatctttattgtctcctttcatctcaacagctgctcgggaatgtcctcgtgtgcgtcctcgctcaccatttcgggaaggagtttacaccccccgtccaagccgcttaccaaaaggtcgtcgccggagtcgccaacgctctcgctcataaataccat

SEQ ID NO: 63 - DNA, 774 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaaggtaggctctggtgaccaggacaagggagggaaggaaggaccctgtgcctggcaaaagtccaggtcgcttctcaggatttgtggcaccttctgactgtcaaactgttcttgtcaatctcacagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaagctctcagctcactatgggttcatctttattgtctcctttcatctcaacagctgctcgggaatgtcctcgtgtgcgtcctcgctcaccatttcgggaaggagtttacaccccccgtccaagccgcttaccaaaaggtcgtcgccggagtcgccaacgctctcgctcataaataccat

SEQ ID NO: 64 - DNA, 874 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaaggtaggctctggtgaccaggattctcaggatttgtggcaccttctgactgtcaaactgttcttgtcaatctcacagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggagctggacacatataaaatgctgctaatgcttcattacaaacttatatcctttaattccagatgggggcaaagtatagctctcagctcactatgggttcatctttattgtctcctttcatctcaacagctgctcgggaatgtcctcgtgtgcgtcctcgctcaccatttcgggaaggagtttacaccccccgtccaagccgcttaccaaaaggtcgtcgccggagtcgccaacgctctcgctcataaataccat

SEQ ID NO: 65 - DNA, 663 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaaggtaggctctggtgaccaggattctcaggatttgtggcaccttctgactgtcaaactgttcttgtcaatctcacagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgtttcgctggacacatataaaatgctgctaatgcttcattacaaacttatatcctttaattccagatgggggcaaagtatagctctcagctcactatgggttcatctttattgtctcctttcatctcaacagctgctcgggaatgtcctcgtgtgcgtcctcgctcaccatttcgggaaggagtttacaccccccgtccaagccgcttaccaaaaggtcgtcgccggagtcgccaacgctctcgctcataaataccat

SEQ ID NO: 66 - DNA, 727 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaaggtaggctctggtgaccaggattctcaggatttgtggcaccttctgactgtcaaactgttcttgtcaatctcacagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaagctctcagctcactatgggttcatctttattgtctcctttcatctcaacagctgctcgggaatgtcctcgtgtgcgtcctcgctcaccatttcgggaaggagtttacaccccccgtccaagccgcttaccaaaaggtcgtcgccggagtcgccaacgctctcgctcataaataccat

SEQ ID NO: 67 - DNA, 1452 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaaggtaggctctggtgaccaggacaagggagggaaggaaggaccctgtgcctggcaaaagtccaggtcgcttctcaggatttgtggcaccttctgactgtcaaactgttcttgtcaatctcacagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgcgcgcgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagcttggtgtgtagttatctggaggccaggctggagctctcagctcactatgggttcatctttattgtctcctttcatctcaacagctgctcgggaatgtcctcgtgtgcgtcctcgctcaccatttcgggaaggagtttacaccccccgtccaagccgcttaccaaaaggtcgtcgccggagtcgccaacgctctcgctcataaataccattaa

SEQ ID NO: 68 - DNA, 713 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaaggtaggctctggtgaccaggacaagggagggaaggaaggaccctgtgcctggcaaaagtccaggtcgcttctcaggatttgtggcaccttctgactgtcaaactgttcttgtcaatctcacagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgtttcgctggacacatataaaatgctgctaatgcttcattacaaacttatatcctttaattccagatgggggcaaagtatagctctcagctcactatgggttcatctttattgtctcctttcatctcaacagctgctcgggaatgtcctcgtgtgcgtcctcgctcaccatttcgggaaggagtttacaccccccgtccaagccgcttaccaaaaggtcgtcgccggagtcgccaacgctctcgctcataaataccattaa

SEQ ID NO: 69 - DNA, 865 bp, Artificial Sequence
  Description of Artificial Sequence Synthetic polynucleotide
taacctcctatttgacaccactgattaccccattgatagtcacactttgggttgtaagtgactttttatttatttgtatttttgactgcattaagaggtctctagttttttacctcttgtttcccaaaacctaataagtaactaatgcacagagcacattgatttgtatttattctatttttagacataatttattagcatgcatgagcaaattaagaaaaacaacaacaaatgaatgcatatatatgtatatgtatgtgtgtacatatacacatatatatatatattttttttcttttcttaccagaaggttttaatccaaataaggagaagatatgcttagaactgaggtagagttttcatccattctgtcctgtaagtattttgcatattctggagacgcaggaagagatccatctacatatcccaaagctgaattatggtagacaaaactcttccacttttagtgcatcaatttcttatttgtgtaataagaaaattgggaaaacgatcttcaatatgcttaccaagctgtgattccaaatattacgtaaatacacttgcaaaggaggatgtttttagtagcaatttgtactgatggtatggggccaagagatatatcttagagggagggctgagggtttgaagtccaactcctaagccagtgccagaagagccaaggacaggtacggctgtcatcacttagacctcaccctgtggagccacaccctagggttggccaatctactcccaggagcagggagggcaggagccagggctgggcataaaagtcagggcagagccatctattgcttacatttgcttctgacacaactgtgttcactagcaacctcaaacagacacc

SEQ ID NO: 70 - DNA, 795 bp, Artificial Sequence
  Description of Artificial Sequence Synthetic polynucleotide
ctgccctgtggggcaaggtgaacgtggatgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagggtgagtctatgggacccttgatgttttctttccccttcttttctatggttaagttcatgtcataggaaggggataagtaacagggtacagtttagaatgggaaacagacgaatgattgcatcagtgtggaagtctcaggatcgttttagtttcttttatttgctgttcataacaattgttttcttttgtttaattcttgctttctttttttttcttctccgcaatttttactattatacttaatgccttaacattgtgtataacaaaaggaaatatctctgagatacattaagtaacttaaaaaaaaactttacacagtctgcctagtacattactatttggaatatatgtgtgcttatttgcatattcataatctccctactttat

SEQ ID NO: 71 - DNA, 1254 bp, Homo sapiens
atgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaa

SEQ ID NO: 72 - DNA, 1520 bp, Artificial Sequence
  Description of Artificial Sequence Synthetic polynucleotide
atgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggtgaggctccctcccctgctccgacccgggctcctcgcccgcccggacccacaggccaccctcaaccgtcctggccccggacccaaaccccacccctcactctgcttctccccgcaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtgagcggcgggccgggagcgatctgggtcgaggggcgagatggcgccttcctcgcagggcagaggatcacgcgggttgcgggaggtgtagcgcaggcggcggctgcgggcctgggccctcggccccactgaccctcttctctgcacaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaa

SEQ ID NO: 73 - DNA, 2262 bp, Artificial Sequence
  Description of Artificial Sequence Synthetic polynucleotide
atgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggtaggctctggtgaccaggacaagggagggaaggaaggaccctgtgcctggcaaaagtccaggtcgcttctcaggatttgtggcaccttctgactgtcaaactgttcttgtcaatctcacaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgcgcgcgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagcttggtgtgtagttatctggaggccaggctggagctctcagctcactatgggttcatctttattgtctcctttcatctcaacaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaa

SEQ ID NO: 74 - DNA, 122 bp, Homo sapiens; identical to SEQ ID NO: 11

SEQ ID NO: 75 - DNA, 75 bp, Artificial Sequence
  Description of Artificial Sequence Synthetic oligonucleotide
gtaggctctggtgaccaggattctcaggatttgtggcaccttctgactgtcaaactgttcttgtcaatctcacag

SEQ ID NO: 76 - DNA, 886 bp, Homo sapiens; identical to SEQ ID NO: 12

SEQ ID NO: 77 - DNA, 358 bp, Artificial Sequence
  Description of Artificial Sequence Synthetic polynucleotide
gtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggagctggacacatataaaatgctgctaatgcttcattacaaacttatatcctttaattccagatgggggcaaagtatagctctcagctcactatgggttcatctttattgtctcctttcatctcaacag

SEQ ID NO: 78 - DNA, 147 bp, Artificial Sequence
  Description of Artificial Sequence Synthetic polynucleotide
gtgagtccaggagatgtttcgctggacacatataaaatgctgctaatgcttcattacaaacttatatcctttaattccagatgggggcaaagtatagctctcagctcactatgggttcatctttattgtctcctttcatctcaacag

SEQ ID NO: 79 - DNA, 211 bp, Artificial Sequence
  Description of Artificial Sequence Synthetic polynucleotide
gtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaagctctcagctcactatgggttcatctttattgtctcctttcatctcaacag

SEQ ID NO: 80 - DNA, 19 bp, Artificial Sequence
  Description of Artificial Sequence Synthetic oligonucleotide
gaggaaaaatccgcagtca

SEQ ID NO: 81 - protein, 147 aa, Homo sapiens
MVHLTPEEKSAVTALWGKVNVDEVGGEALGRLLVVYPWTQRFFESFGDLSTPDAVMGNPKVKAHGKKVLGAFSDGLAHLDNLKGTFATLSELHCDKLHVDPENFRLLGNVLVCVLAHHFGKEFTPPVQAAYQKVVAGVANALAHKYH

SEQ ID NO: 82 - protein, 30 aa, Homo sapiens
MVHLTPEEKSAVTALWGKVNVDEVGGEALG

SEQ ID NO: 83 - protein, 74 aa, Homo sapiens
LLVVYPWTQRFFESFGDLSTPDAVMGNPKVKAHGKKVLGAFSDGLAHLDNLKGTFATLSELHCDKLHVDPENFR

SEQ ID NO: 84 - protein, 42 aa, Homo sapiens
LLGNVLVCVLAHHFGKEFTPPVQAAYQKVVAGVANALAHKYH

SEQ ID NO: 85 - DNA, 223 bp, Homo sapiens
gctgctggtggtctacccttggacccagaggttctttgagtcctttggggatctgtccactcctgatgctgttatgggcaaccctaaggtgaaggctcatggcaagaaagtgctcggtgcctttagtgatggcctggctcacctggacaacctcaagggcacctttgccacactgagtgagctgcactgtgacaagctgcacgtggatcctgagaacttcagg

SEQ ID NO: 86 - DNA, 126 bp, Homo sapiens
ctcctgggcaacgtgctggtctgtgtgctggcccatcactttggcaaagaattcaccccaccagtgcaggctgcctatcagaaagtggtggctggtgtggctaatgccctggcccacaagtatcac

SEQ ID NO: 87 - DNA, 1421 bp, Homo sapiens; identical to SEQ ID NO: 6

SEQ ID NO: 88 - DNA, 1660 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaaggtaggctctggtgaccaggacaagggagggaaggaaggaccctgtgcctggcaaaagtccaggtcgcttctcaggatttgtggcaccttctgactgtcaaactgttcttgtcaatctcacagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgcgcgcgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagcttggtgtgtagttatctggaggccaggctggagctctcagctcactatgggttcatctttattgtctcctttcatctcaacagctgctcgggaatgtcctcgtgtgcgtcctcgctcaccatttcgggaaggagtttacaccccccgtccaagccgcttaccaaaaggtcgtcgccggagtcgccaacgctctcgctcataaataccattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 89 - DNA, 921 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaaggtaggctctggtgaccaggacaagggagggaaggaaggaccctgtgcctggcaaaagtccaggtcgcttctcaggatttgtggcaccttctgactgtcaaactgttcttgtcaatctcacagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgtttcgctggacacatataaaatgctgctaatgcttcattacaaacttatatcctttaattccagatgggggcaaagtatagctctcagctcactatgggttcatctttattgtctcctttcatctcaacagctgctcgggaatgtcctcgtgtgcgtcctcgctcaccatttcgggaaggagtttacaccccccgtccaagccgcttaccaaaaggtcgtcgccggagtcgccaacgctctcgctcataaataccattaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 90 - DNA, 1574 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaaggtaggctctggtgaccaggacaagggagggaaggaaggaccctgtgcctggcaaaagtccaggtcgcttctcaggatttgtggcaccttctgactgtcaaactgttcttgtcaatctcacagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgcgcgcgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagcttggtgtgtagttatctggaggccaggctggagctctcagctcactatgggttcatctttattgtctcctttcatctcaacagctgctcgggaatgtcctcgtgtgcgtcctcgctcaccatttcgggaaggagtttacaccccccgtccaagccgcttaccaaaaggtcgtcgccggagtcgccaacgctctcgctcataaataccattaaaacttgtttattgcagcttataatggttacaaataaagcaatagcatcacaaatttcacaaataaagcatttttttcactgcattctagttgtggtttgtccaaactcatcaatgtatctta

SEQ ID NO: 91 - DNA, 835 bp, Artificial Sequence
  Description of Artificial Sequence Synthetic polynucleotide
atggtccacctcacccccgaagaaaaatccgcagtcaccgctctctggggaaaagtcaatgtcgacgaggtgggaggcgaagctctcggaaggtaggctctggtgaccaggacaagggagggaaggaaggaccctgtgcctggcaaaagtccaggtcgcttctcaggatttgtggcaccttctgactgtcaaactgttcttgtcaatctcacagactcctcgtcgtgtatccctggacacaaagatttttcgaaagcttcggcgacctcagcacacccgacgccgtgatggggaatcccaaagtcaaagcccacgggaaaaaggtcctgggcgctttctccgacggactcgcccatctcgataatctgaaaggaactttcgctaccctctccgaactccattgcgataaactccatgtcgaccccgaaaattttagagtgagtccaggagatgtttcgctggacacatataaaatgctgctaatgcttcattacaaacttatatcctttaattccagatgggggcaaagtatagctctcagctcactatgggttcatctttattgtctcctttcatctcaacagctgctcgggaatgtcctcgtgtgcgtcctcgctcaccatttcgggaaggagtttacaccccccgtccaagccgcttaccaaaaggtcgtcgccggagtcgccaacgctctcgctcataaataccattaaaacttgtttattgcagcttataatggttacaaataaagcaatagcatcacaaatttcacaaataaagcatttttttcactgcattctagttgtggtttgtccaaactcatcaatgtatctta

SEQ ID NO: 92 - RNA, 100 nt, Artificial Sequence
  Description of Artificial Sequence Synthetic polynucleotide
  modified_base (1)..(3): Nucleotide with 2′-O-methyl modification and MS Modification in the phosphate backbone
  modified_base (97)..(99): Nucleotide with 2′-O-methyl modification and MS Modification in the phosphate backbone
cuugccccacagggcaguaaguuuuagagcuagaaauagcaaguuaaaauaaggcuaguccguuaucaacuugaaaaaguggcaccgagucggugcuuuu

SEQ ID NO: 93 - DNA, 646 bp, Homo sapiens
atgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaag

SEQ ID NO: 94 - DNA, 271 bp, Homo sapiens
gcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaag

SEQ ID NO: 95 - DNA, 337 bp, Homo sapiens
gtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaa

SEQ ID NO: 96 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 97 - protein, 30 aa, Artificial Sequence; identical to SEQ ID NO: 82
  Description of Artificial Sequence Synthetic polypeptide